SEQUENCE LISTING (13 sequences)

SEQ ID NO: 1 - DNA, 12 bp, Artificial Sequence
  biotinylated target probe
  misc_feature 1: B-heg
  misc_feature 12: dT
atgcacagtcgg

SEQ ID NO: 2 - DNA, 23 bp, Artificial Sequence
  biotinylated target probe
  misc_feature (12)...(13): heg-F-(heghegheg-F)3-heg
  misc_feature 23: dT
cgacgcttacagccgactgtgca

SEQ ID NO: 3 - DNA, 24 bp, Artificial Sequence
  biotinylated target probe
  misc_feature (12)...(13): heg-F(heghegheg-F)9-heg
  misc_feature 24: dT
ctgtaagcgtcggacattcgcagc

SEQ ID NO: 4 - DNA, 21 bp, Artificial Sequence
  biotinylated target probe
  misc_feature 1: Biotin-heg
  misc_feature 21: dT
tttctttgaggtttaggattc

SEQ ID NO: 5 - DNA, 61 bp, Artificial Sequence
  biotinylated target probe
  misc_feature 1: dT
  misc_feature 61: heg-F
aaagaaactccaaatcctaagggttggccttagggttcagaggttggccttagggttcaga

SEQ ID NO: 6 - DNA, 17 bp, Artificial Sequence
  biotinylated target probe
  misc_feature 17: X-F-dT
ccaaccggaatcccaag

SEQ ID NO: 7 - DNA, 17 bp, Artificial Sequence
  biotinylated target probe
  misc_feature 17: heg-F(heghegheg-F)3-heg-dT
ccaaccggaatcccaag

SEQ ID NO: 8 - DNA, 17 bp, Artificial Sequence
  biotinylated target probe
  misc_feature 17: heg-F(heghegheg-F)4-heg-dT
ccaaccggaatcccaag

SEQ ID NO: 9 - DNA, 17 bp, Artificial Sequence
  biotinylated target probe
  misc_feature 17: heg-F(heghegheg-F)9-heg-dT
ccaaccggaatcccaag

SEQ ID NO: 10 - DNA, 24 bp, Artificial Sequence
  biotinylated target probe
  misc_feature 1: d
  misc_feature (12)...(13): heg-F(heghegheg-F)3-heg
tacgtgtcagccgacattcgcagc

SEQ ID NO: 11 - DNA, 24 bp, Artificial Sequence
  biotinylated target probe
  misc_feature (12)...(13): heg-F(heghegheg-F)4-heg
  misc_feature 23    24: dT
ctgtaagcgtcgcgacgcttacag

SEQ ID NO: 12 - DNA, 37 bp, Artificial Sequence
  biotinylated target probe
  misc_feature 1: dT
  misc_feature (20)...(21): heg-F(heghegheg-F)-heg
aaagaaactccaatcctaagggttggccttagggttc

SEQ ID NO: 13 - DNA, 33 bp, Artificial Sequence
  biotinylated target probe
  misc_feature (17)...(18): heg-F(heghegheg-F)-heg
  misc_feature 33: dT
ccaaccggaatcccaagctttgaggtttaggat

(19) United States
(12) Patent Application Publication
(10) Pub. No.: US 2024/0092820 A1
(43) Pub. Date: Mar. 21, 2024

(54) POLYMER DYES

(71) Applicant: Sony Group Corporation, Tokyo (JP)

(72) Inventors: Tracy Matray, Snohomish, WA (US); Sharat Singh, Rancho Santa Fe, CA (US)

(21) Appl. No.: 18/254,030

(22) PCT Filed: Nov. 22, 2021
(86) PCT No.: PCT/US2021/060378

§ 371 Date: May 23, 2023

Related U.S. Application Data
(60) Provisional application No. 63/118,544, filed on Nov. 25, 2020.

(51) Int. Cl. C07H 21/00; G01N 33/58

(52) U.S. Cl. CPC C07H 21/00; G01N 33/58

(57) ABSTRACT

Compounds useful as probes are disclosed. The compounds have the following structure (II): or a stereoisomer, tautomer or salt thereof, wherein M, L^{1a}, L^{1b}, L², L³, L⁴, L⁵, L⁶, L⁷, L⁸, L⁹-R¹, R², R³, R⁴, R⁵, m, n, q, and w areas defined herein. Additionally, compositions, kits, and methods useful for detecting a target analyte are also described.

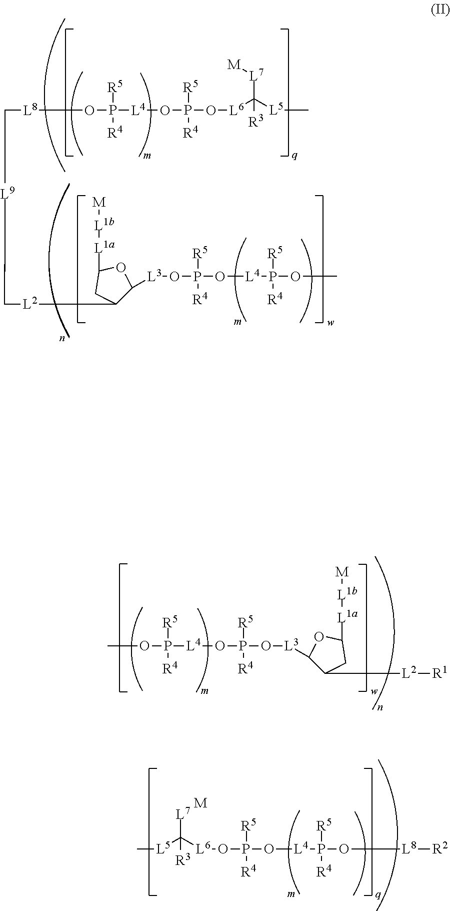

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 870268_430WO_SEQUENCE_LISTING.txt. The text file is 4.1 KB, was created on Nov. 19, 2021, and is being submitted electronically via EFS-Web.

### BACKGROUND

### Technical Field

The present disclosure is generally directed to compounds comprising polymeric chromophores covalently bound to at least one polynucleotide (e.g., compounds comprising polymer fluorophore moieties bound to a nucleotide probe), as well as compositions and kits comprising the same, and methods for their preparation and use in various analytical methods.

### Description of the Related Art

Probes are used to detect specific target sequences or other analytes in various diagnostic and analytical contexts. For example, conventional, heterogeneous, hybridization assays typically comprise the following steps: immobilization of a target nucleic acid (e.g., on paper, beads, or plastic surfaces); addition of labelled probes that are complementary to the sequence of the target; hybridization; removal of unhybridized probes; and detection of the probes remaining bound to the immobilized target. However, using solid surfaces to immobilize the target nucleic acids lengthens the time it takes for hybridization by restricting the mobility of, or access to, the target by the probes. Additionally, solid surfaces may interfere with signal from the probes or lead to noise in the signal. The requirement that the probe-target hybrids be isolated also precludes in vivo detection and concurrent detection of nucleic acids during synthesis reactions (real-time detection).

There is therefore a need in the art for improved probes that with increased brightness and produce a lower signal-to-noise ratio. The present disclosure fulfills this need and provides further related advantages.

### BRIEF SUMMARY

In one aspect, described herein are compounds having the following structure (II):

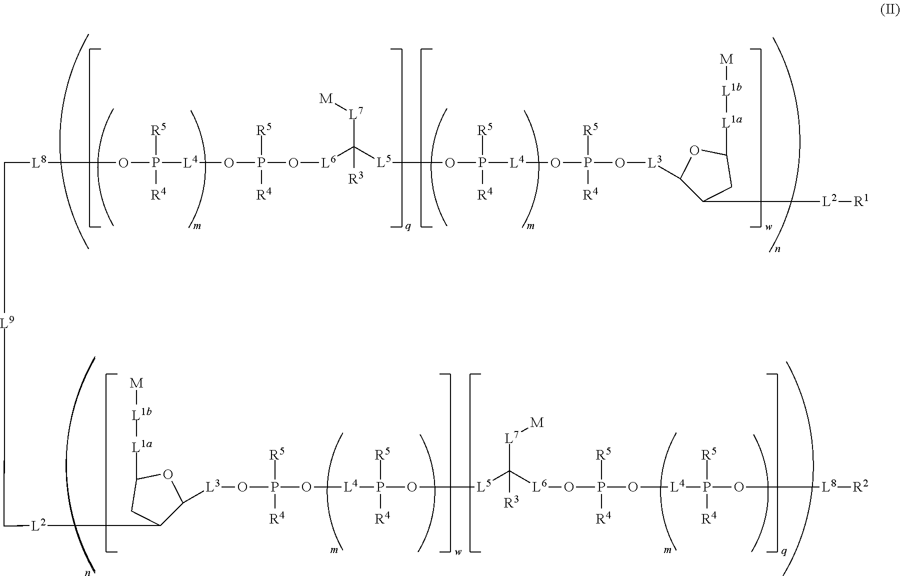

or a stereoisomer, salt or tautomer thereof, wherein:
M is, at each occurrence, independently a chromophore;
L^{1a} is, at each occurrence, independently a heteroarylene linker;
L² and L⁸ are independently optional linkers;
L^{1b}, L³, L⁵, L⁶ and L⁷ are, at each occurrence, independently optional alkylene, alkenylene, alkynylene, heteroalkylene, heteroalkenylene or heteroalkynylene linkers;
L⁴ is, at each occurrence, independently an alkylene, alkenylene, alkynylene, heteroalkylene, heteroalkenylene or heteroalkynylene linker;
L⁹ is a linker comprising a polynucleotide;
R¹ and R² are each independently H, OH, SH, alkyl, alkoxy, alkylether, heteroalkyl or —OP(═Rₐ)(R_{b})R_{c};
R³ is, at each occurrence, independently H, alkyl or alkoxy;
R⁴ is, at each occurrence, independently OH, SH, O⁻, S⁻, OR_{d} or SR_{d};
R⁵ is, at each occurrence, independently oxo, thioxo or absent;
Rₐ is O or S;
R_{b} is OH, SH, O⁻, S⁻, OR_{d} or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkyl ether or thiophosphoalkyl ether;
R_{d} is a counter ion;
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of one or greater; and
q and w are, at each occurrence, independently 0 or 1, provided at least one occurrence of either q or w is 1.

In another aspect, the present disclosure provides a compound having the following structure (III);

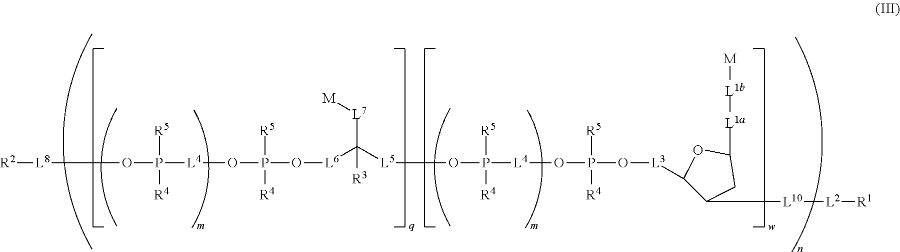

or a stereoisomer, salt or tautomer thereof, wherein:
M is, at each occurrence, independently the same or different chromophore;
L^{1a} is, at each occurrence, independently a heteroarylene linker;
L² and L⁸ are independently optional linkers;
L^{1b}, L³, L⁵, L⁶ and L⁷ are, at each occurrence, independently optional alkylene, alkenylene, alkynylene, heteroalkylene, heteroalkenylene or heteroalkynylene linkers;
L⁴ is, at each occurrence, independently an alkylene, alkenylene, alkynylene, heteroalkylene, heteroalkenylene or heteroalkynylene linker;
L¹⁰ is a linker comprising a polynucleotide;
R¹ and R² each independently H, OH, SH, alkyl, alkoxy, alkyl ether, heteroalkyl, —OP(═Rₐ)(R_{b})R_{c} or a moiety comprising a polynucleotide, provided at least one of R¹ and R² is a moiety comprising a polynucleotide;
R³ is, at each occurrence, independently H, alkyl or alkoxy;
R⁴ is; at each occurrence, independently OH, SH, O⁻, S⁻, OR_{d} or SR_{d};
R⁵ is, at each occurrence, independently oxo, thioxo or absent;
Rₐ is O or S;
R_{b} is OH, SH, O⁻, S⁻, OR_{d} or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;
R_{d} s a counter ion;
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of two or greater; and
q and w are, at each occurrence, independently 0 or 1, provided at least one occurrence of either q or w is 1.

In a further aspect, described herein is a composition comprising a compound of structure (II) and a capture probe.

Also described herein is a composition comprising a compound of structure (II) and a compound of structure (III).

In still a further aspect, described herein is a composition comprising a compound of structure (III) and a compound having the following structure (I):

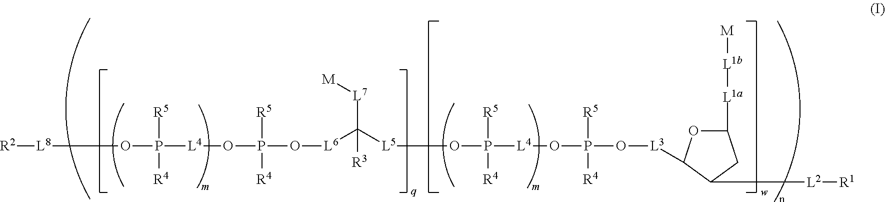

or a stereoisomer, salt or tautomer thereof, wherein:
M is, at each occurrence, independently the same or different chromophore;
L^{1a} is, at each occurrence, independently a heteroarylene linker;
L² and L⁸ are independently optional linkers;
L^{1b}, L³, L⁵, L⁶ and L⁷ are, at each occurrence, independently optional alkylene, alkenylene, alkynylene, heteroalkylene, heteroalkenylene or heteroalkynylene linkers;
L⁴ is, at each occurrence, independently an alkylene, alkenylene, alkynylene, heteroalkylene, heteroalkenylene or heteroalkynylene linker;
R¹ and R² each independently H; OH, SH, alkyl, alkoxy, alkylether, heteroalkyl, —OP(═Rₐ)(R_{b})R_{c} or a moiety comprising a polynucleotide, provided at least one of R¹ and R² is a moiety comprising a polynucleotide;
R³ is, at each occurrence, independently H, alkyl or alkoxy;
R⁴ is, at each occurrence, independently OH, SH, O⁻, S⁻, OR_{d} or SR_{d};
R⁵ is, at each occurrence, independently oxo, thioxo or absent;
Rₐ O or S;
R_{b} is OH, SH, O⁻, S⁻, OR_{d} or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether,
R_{d} is a counter ion;
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of one or greater; and
q and w are, at each occurrence, independently 0 or 1, provided at least one occurrence of either q or w is 1.

Also described herein is a composition comprising: (1) a capture probe comprising: (i) a first segment comprising a first polynucleotide having a first sequence; and (ii) a second segment conjugated to the first segment, the second segment comprising a plurality of alternating second polynucleotides and first polymers, each of the first polymers comprising a first chromophore, each of the second polynucleotides comprising a second sequence; and (2) a plurality of detectable probes, each of the plurality of detectable probes comprising a third polynucleotide covalently bound to at least one a second polymer comprising a second chromophore, the third polynucleotide having a third sequence that has at least 90% complementarity to the second sequence.

In yet another aspect, described herein is a composition comprising: (1) a capture probe comprising: (i) a first segment comprising a first polynucleotide having a first sequence; and (ii) a second segment conjugated to the first segment, the second segment comprising a plurality of second polynucleotides each comprising a second sequence; and (2) a plurality of detectable probes, each of the plurality of detectable probes comprising a third polynucleotide having a first end and a second end, the first end being covalently bound to a first polymer comprising an acceptor chromophore, the second end being covalently bound to a second polymer comprising a donor chromophore, the third polynucleotide having a third sequence that has at least 90% complementarity to the second sequence.

Additionally, described herein is a kit comprising a compound of composition described herein.

In still a further embodiment, the present disclosure provides a method for identifying the presence of a target analyte, comprising: producing a mixture by contacting a sample with a composition as described herein under assay conditions; and imaging the mixture under detection conditions

### BRIEF DESCRIPTION OF THE SEVERAL VIEWS OF THE DRAWINGS

This patent or application file contains at least one drawing executed in color. Copies of this patent or patent application publication with color drawing(s) will be provided by the Office upon request and payment of the necessary fee.

In the figures, identical reference numbers identify similar elements. The sizes and relative positions of elements in the figures are not necessarily drawn to scale and some of these elements are enlarged and positioned to improve figure legibility. Further, the particular shapes of the elements as drawn are not intended to convey any information regarding the actual shape of the particular elements, and have been solely selected for ease of recognition in the figures.

FIG. 1 shows a schematic representation of an embodiment of a probe complex.

FIG. 2A shows a schematic representation of another embodiment of a probe complex.

FIG. 2B shows a representation of a further embodiment of a probe complex.

FIG. 2C shows a representation of a further embodiment of a probe complex.

FIG. 3A shows a representation of a further embodiment of a probe complex.

FIG. 3B shows a representation of a further embodiment of a probe complex.

FIG. 4 shows the results of the testing described in Example 3.

FIG. 5 shows the results of the testing described in Example 5.

FIG. 6 shows the results of the testing described in Example 6.

FIG. 7 illustrates an embodiment of the concatemerization of probes as described in Example 7.

FIG. 8A shows the results of the testing described in Example 8.

FIG. 8B shows the results of the testing described in Example 8.

### DETAILED DESCRIPTION

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Amino” refers to the —NH₂ group.

“Carboxy” refers to the —CO₂H group.

“Cyano” refers to the —CN group.

“Formyl” refers to the —C(═O)H group.

“Hydroxy” or “hydroxyl” refers to the —OH group.

“Imino” refers to the ═NH group.

“Nitro” refers to the —NO₂ group.

“Oxo” refers to the ═O substituent group.

“Sulfhydryl.” refers to the —SH group.

“Thioxo” refers to the ═S group.

“Alkyl” refers to a straight or branched hydrocarbon chain group consisting solely of carbon and hydrogen atoms, containing no unsaturation, having from one to twelve carbon atoms (C₁-C₁₂ alkyl), one to eight carbon atoms (C₁-C₈ alkyl) or one to six carbon atoms (C₁-C₆ alkyl), and which is attached to the rest of the molecule by a single bond. e.g., methyl, ethyl, n-propyl, 1 methyl ethyl (iso-propyl), n-butyl, n-pentyl, 1,1-dimethylethyl (t-butyl), 3-methylhexyl, 2-methylhexyl, and the like. Unless stated otherwise specifically in the specification, alkyl groups are optionally substituted.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, alkylene is optionally substituted.

“Alkenylene” or “alkenylene chain” refers to a straight or branched divalent hydrocarbon chain linking the rest of the molecule to a radical group, consisting solely of carbon and hydrogen, containing at least one carbon-carbon double bond and having from two to twelve carbon atoms, e.g., ethenylene, propenylene, n butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, alkenylene is optionally substituted.

“Alkynylene” or “alkynylene chain” refers to a straight or branched divalent hydrocarbon chain linking the rest of the molecule to a radical group, consisting solely of carbon and hydrogen, containing at least one carbon-carbon triple bond and having from two to twelve carbon atoms, e.g., ethenylene, propenylene, n butenylene, and the like. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain. Unless stated otherwise specifically in the specification, alkynylene is optionally substituted.

“Alkylether” refers to any alkyl group as defined above, wherein at least one carbon-carbon bond is replaced with a carbon-oxygen bond. The carbon-oxygen bond may be on the terminal end (as in an alkoxy group) or the carbon oxygen bond may be internal (i.e., C—O—C). Alkylethers include at least one carbon oxygen bond, but may include more than one. For example, polyethylene glycol (PEG) is included within the meaning of alkylether. Unless stated otherwise specifically in the specification, an alkylether group is optionally substituted. For example, in some embodiments an alkylether is substituted with an alcohol or —OP(═Rₐ)(R_{b})R_{c}, wherein each of Rₐ, R_{b} and R_{c} is as defined for compounds of structure (I).

“Alkoxy” refers to a group of the formula —ORₐ where Rₐ is an alkyl group as defined above containing one to twelve carbon atoms. Unless stated otherwise specifically in the specification, an alkoxy group is optionally substituted.

“Alkoxyalkylether” refers to a group of the formula —ORₐR_{b} where Rₐ is an alkylene group as defined above containing one to twelve carbon atoms, and R_{b} is an alkylether group as defined herein. Unless stated otherwise specifically in the specification, an alkoxyalkylether group is optionally substituted, for example substituted with an alcohol or —OP(═Rₐ)(R_{b})R_{c}, wherein each of Rₐ, R_{b} and R_{c} is as defined for compounds of structure (I).

“Heteroalkyl” refers to an alkyl group, as defined above, comprising at least one heteroatom (e.g., N, O, P, or S) within the alkyl group or at a terminus of the alkyl group. In some embodiments, the heteroatom is within the alkyl group (i.e., the heteroalkyl comprises at least one carbon[heteroatom]-carbon bond, where x is 1, 2 or 3). In other embodiments, the heteroatom is at a terminus of the alkyl group and thus serves to join the alkyl group to the remainder of the molecule (e.g., M₁-H-A), where M₁ is a portion of the molecule, H is a heteroatom, and A is an alkyl group). Unless stated otherwise specifically in the specification, a heteroalkyl group is optionally substituted. Exemplary groups include ethylene oxide (e.g., polyethylene oxide), optionally including phosphorous-oxygen bonds, such as phosphodiester bonds.

“Heteroalkoxy” refers to a group of the formula —ORₐ where Rₐ is a heteroalkyl group as defined above containing one to twelve carbon atoms. Unless stated otherwise specifically in the specification, a heteroalkoxy group is optionally substituted.

“Heteroalkylene” refers to an alkylene group, as defined above, comprising at least one heteroatom (e.g., N, O, P or S) within the alkylene chain or at a terminus of the alkylene chain. In some embodiments, the heteroatom is within the alkylene chain (i.e., the heteroalkylene comprises at least one carbon-[heteroatom]-carbon bond, where x is 1, 2 or 3). In other embodiments, the heteroatom is at a terminus of the alkylene and thus serves to join the alkylene to the remainder of the molecule (e.g., M₁-H-A-M₂, where M₁ and M₂ are portions of the molecule, H is a heteroatom and A is an alkylene). Unless stated otherwise specifically in the specification, a heteroalkylene group is optionally substituted. Exemplary heteroalkylene groups include ethylene oxide (e.g., polyethylene oxide) and the “C,” “HEG,” “TEG,” “PEG 1K” and variations thereof, linking groups illustrated below:

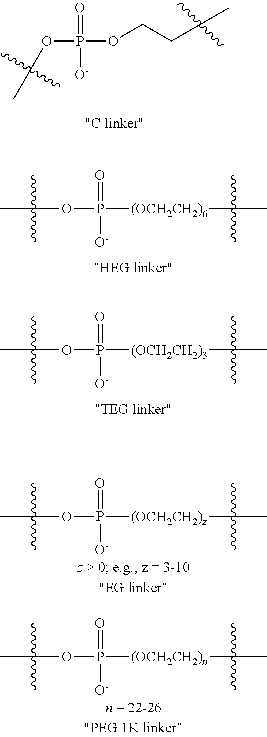

Multimers of the above C-linker, HEG linker and/or PEG 1K linker are included in various embodiments of heteroalkylene linkers.

In some embodiments of the PEG 1K linker, n is 25. Multimers may comprise, for example, the following structure:

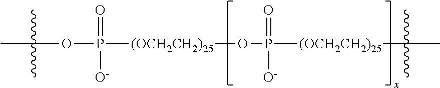

wherein x is 0 or an integer greater than 0, for example, x ranges from 0-1.00 (e.g., 1, 2, 3, 4, 5, 6, 7, 8, 9, or 10).

“Heteroalkenylene” is a heteroalkylene, as defined above, comprising at least one carbon-carbon double bond. Unless stated otherwise specifically in the specification, a heteroalkenylene group is optionally substituted.

“Heteroalkynylene” is a heteroalkylene comprising at least one carbon-carbon triple bond. Unless stated otherwise specifically in the specification, a heteroalkynylene group is optionally substituted.

“Heteroatomic” in reference to a “heteroatomic linker” refers to a linker group consisting of one or more heteroatoms. Exemplary heteroatomic linkers include single atoms selected from the group consisting of O, N, P and S, and multiple heteroatoms for example a linker having the formula —P(O—)(═O)O— or —OP(O—)(═O)O— and multimers and combinations thereof.

“Phosphate” refers to the —OP(═O)(Rₐ)R_{b} group, wherein Rₐ is OH, O— or OR_{c}; and R_{b} is OH, O—, OR_{c}, a thiophosphate group or a further phosphate group, wherein Re is a counter ion (e.g., Na+ and the like).

“Phosphoalkyl” refers to the —OP(═O)(Rₐ)R_{b} group, wherein Rₐ is OH, O— or ORc; and R_{b} is —Oalkyl, wherein R_{c} is a counter ion (e.g., Na+ and the like). Unless stated otherwise specifically in the specification, a phosphoalkyl group is optionally substituted. For example, in certain embodiments, the —Oalkyl moiety in a phosphoalkyl group is optionally substituted with one or more of hydroxyl, amino, sulfhydryl, phosphate, thiophosphate, phosphoalkyl, thiophosphoalkyl, phosphoalkylether, thiophosphoalkylether or —OP(—Rₐ)(R_{b})R_{c}, wherein each of Rₐ, R_{b} and R_{c} is as defined for compounds of structure (I).

“Phosphoalkylether” refers to the —OP(═O)(Rₐ)R_{b} group, wherein Rₐ is OH, O— or OR_{c}; and R_{b} is —Oalkylether, wherein R_{c} is a counter ion (e.g., Na+ and the like). Unless stated otherwise specifically in the specification, a phosphoalkylether group is optionally substituted. For example, in certain embodiments, the —Oalkylether moiety in a phosphoalkylether group is optionally substituted with one or more of hydroxyl, amino, sulfhydryl, phosphate, thiophosphate, phosphoalkyl, thiophosphoalkyl, phosphoalkylether, thiophosphoalkylether or —OP(—Rₐ)(R_{b})R_{c}, wherein each of Rₐ, R_{b} and R_{c} is as defined for compounds of structure (I).

“Thiophosphate” refers to the —OP(═Rₐ)(R_{b})R_{c} group, wherein Rₐ is O or S. R_{b} is OH, O—, S—, OR_{d} or SR_{d}; and R_{c} is OH, SH, O—, S—, OR_{d}, SR_{d}, a phosphate group or a further thiophosphate group, wherein R_{d} is a counter ion (e.g., Na+ and the like) and provided that: i) Rₐ is S; ii) R_{b} is S— or SR_{d}; iii) R_{c} is SH, S— or SR_{d}; or iv) a combination of i), ii) and/or iii).

“Thiophosphoalkyl” refers to the —OP(═Rₐ)(R_{b})R_{c} group, wherein Rₐ is O or S, R_{b} is OH, O—, S—, OR_{d} or SR_{d}; and R_{c} is —Oalkyl, wherein R_{d} is a counter ion (e.g., Na+ and the like) and provided that: i) Rₐ is S; ii) R_{b} is S— or SR_{d}; or iii)Rₐ is S and R_{b} is S— or SR_{d}. Unless stated otherwise specifically in the specification, a thiophosphoalkyl group is optionally substituted. For example, in certain embodiments, the —Oalkyl moiety in a thiophosphoalkyl group is optionally substituted with one or more of hydroxyl, amino, sulfhydryl, phosphate, thiophosphate, phosphoalkyl, thiophosphoalkyl, phosphoalkylether, thiophosphoalkylether or —OP(═Rₐ)(R_{b})R_{c}, wherein each of Rₐ, R_{b} and R_{c} is as defined for compounds of structure (I).

“Thiophosphoalkylether” refers to the —OP(═Rₐ)(R_{b})R_{c} group, wherein Rₐ is O or S, R_{b} is OH, O—, S—, OR_{d} or SR_{d}; and R_{c} is —Oalkylether, wherein R_{d} is a counter ion (e.g., Na+ and the like) and provided that: i) Rₐ is S; ii) R_{b} is S— or SR_{d}; or iii)Rₐ is S and R_{b} is S— or SR_{d}. Unless stated otherwise specifically in the specification, a thiophosphoalkylether group is optionally substituted. For example, in certain embodiments, the —Oalkylether moiety in a thiophosphoalkyl group is optionally substituted with one or more of hydroxyl, amino, sulfhydryl, phosphate, thiophosphate, phosphoalkyl, thiophosphoalkyl, phosphoalkylether, thiophosphoalkylether or —OP(═Rₐ)(R_{b})R_{c}, wherein each of Rₐ, R_{b} and R_{c} is as defined for compounds of structure (I).

“Carbocyclic” refers to a stable 3 to 18 membered aromatic or non-aromatic ring comprising 3 to 18 carbon atoms. Unless stated otherwise specifically in the specification, a carbocyclic ring may be a monocyclic, bicyclic, tricyclic or tetracyclic ring system, which may include fused or bridged ring systems, and may be partially or fully saturated. Non-aromatic carbocyclyl radicals include cycloalkyl, while aromatic carbocyclyl radicals include aryl. Unless stated otherwise specifically in the specification, a carbocyclic group is optionally substituted.

“Cycloalkyl” refers to a stable non-aromatic monocyclic or polycyclic carbocyclic ring, which may include fused or bridged ring systems, having from three to fifteen carbon atoms, preferably having from three to ten carbon atoms, and which is saturated or unsaturated and attached to the rest of the molecule by a single bond. Monocyclic cyclocalkyls include, for example, cyclopropyl, cyclobutyl, cyclopentyl, cyclohexyl, cycloheptly, and cyclooctyl. Polycyclic cycloalkyls include, for example, adamantyl, norbornyl, decalinyl, 7,7 dimethyl-bicyclo-[2.2.1]heptanyl, and the like. Unless stated otherwise specifically in the specification, a cycloalkyl group is optionally substituted.

“Aryl” refers to a ring system comprising at least one carbocyclic aromatic ring. In some embodiments, an aryl comprises from 6 to 18 carbon atoms. The aryl ring may be a monocyclic, bicyclic, tricyclic or tetracyclic ring system, which may include fused or bridged ring systems. Aryls include, for example, aryls derived from aceanthrylene, acenaphthylene, acephenanthrylene, anthracene, azulene, benzene, chrysene, fluoranthene, fluorene, as-indacene, s indacene, indane, indene, naphthalene, phenalene, phenanthrene, pleiadene, pyrene, and triphenylene. Unless stated otherwise specifically in the specification, an aryl group is optionally substituted.

“Heterocyclic” refers to a stable 3 to 18 membered aromatic or non aromatic ring comprising one to twelve carbon atoms and from one to six heteroatoms selected from the group consisting of nitrogen, oxygen and sulfur. Unless stated otherwise specifically in the specification, the heterocyclic ring may be a monocyclic, bicyclic, tricyclic or tetracyclic ring system, which may include fused or bridged ring systems; and the nitrogen, carbon or sulfur atoms in the heterocyclic ring may be optionally oxidized; the nitrogen atom may be optionally quaternized; and the heterocyclic ring may be partially or fully saturated. Examples of aromatic heterocyclic rings are listed below in the definition of heteroaryls (i.e., heteroaryl being a subset of heterocyclic). Examples of non-aromatic heterocyclic rings include, for example, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, pyrazolopyrimidinyl, quinuclidinyl, thiazolidinyl, tetrahydrofuryl, trioxanyl, trithianyl, triazinanyl, tetrahydropyranyl, thiomorpholinyl, thiamorpholinyl, 1 oxo thiomorpholinyl, and 1,1 dioxo thiomorpholinyl. Unless stated otherwise specifically in the specification, a heterocyclic group is optionally substituted.

“Heteroaryl” refers to a 5 to 14 membered ring system comprising one to thirteen carbon atoms, one to six heteroatoms selected from the group consisting of nitrogen, oxygen and sulfur, and at least one aromatic ring. For purposes of certain embodiments of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, for example, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4 benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2 a]pyridinyl, benzoxazolinonyl, benzimidazolthionyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2 oxoazepinyl, oxazolyl, oxiranyl, 1 oxidopyridinyl, 1 oxidopyrimidinyl, 1-oxidopyrazinyl, 1-oxidopyridazinyl, 1 phenyl 1H pyrrolyl, phenazinyl, phenothiazinyl, phenoxazinyl, phthalazinyl, pteridinyl, pteridinonyl, purinyl, pyrrolyl, pyrazolyl, pyridinyl, pyridinonyl, pyrazinyl, pyrimidinyl, pryrimidinonyl, pyridazinyl, pyrrolyl, pyrido[2,3-d]pyrimidinonyl, quinazolinyl, quinazolinonyl, quinoxalinyl, quinoxalinonyl, quinolinyl, isoquinolinyl, tetrahydroquinolinyl, thiazolyl, thiadiazolyl, thieno[3,2-d]pyrimidin-4-onyl, thieno[2,3-d]pyrimidin-4-onyl, triazolyl, tetrazolyl, triazinyl, and thiophenyl (i.e. thienyl). Unless stated otherwise specifically in the specification, a heteroaryl group is optionally substituted.

“Fused” refers to a ring system comprising at least two rings, wherein the two rings share at least one common ring atom, for example two common ring atoms. When the fused ring is a heterocyclyl ring or a heteroaryl ring, the common ring atom(s) may be carbon or nitrogen. Fused rings include bicyclic, tricyclic, tertracyclic, and the like.

The term “substituted” used herein means any of the above groups (e.g., alkyl, alkylene, alkenylene, alkynylene, heteroalkylene, heteroalkenylene, heteroalkynylene, alkoxy, alkylether, alkoxyalkylether, heteroalkyl, heteroalkoxy, phosphoalkyl, phosphoalkylether, thiophosphoalkyl, thiophosphoalkylether, carbocyclic, cycloalkyl, aryl, heterocyclic and/or heteroaryl) wherein at least one hydrogen atom. (e.g., 1, 2, 3 or all hydrogen atoms) is replaced by a bond to a non-hydrogen atoms such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NRgRh, —NRgC(O)Rh, —NRgC(═O)NRgRh, —NRgC(═O)ORh, —NRgSO2Rh, —OC(═O)NRgRh, —ORg, —SRg, —SORg, —SO2Rg, —OSO2Rg, —SO2ORg, =NSO2Rg, and —SO2NRgRh. “Substituted also means any of the above groups in which one or more hydrogen atoms are replaced with —C(═O)Rg, —C(═O)ORg, —C(═O)NRgRh, —CH2SO2Rg, —CH2SO2NRgRh. In the foregoing, Rg and Rh are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkoxy, al amino, thioalkyl, aryl, aralkyl, cycloalkyl, cycloalkylalkyl, haloalkyl, heterocyclyl, N-heterocyclyl, heterocyclyl alkyl, heteroaryl, N-heteroaryl and/or heteroarylalkyl group. In some embodiments, the optional substituent is —OP(═Rₐ)(R_{b})R_{c}, wherein each of Rₐ, Rₕ and R_{c} is as defined for compounds of Structure (I). In addition, each of the foregoing substituents may also be optionally substituted with one or more of the above substituents.

“Conjugation” refers to the overlap of one p-orbital with another p-orbital across an intervening sigma bond. Conjugation may occur in cyclic or acyclic compounds. A “degree of conjugation” refers to the overlap of at least one p-orbital with another p-orbital across an intervening sigma bond. For example, 3-butadine has one degree of conjugation, while benzene and other aromatic compounds typically have multiple degrees of conjugation. Fluorescent and colored compounds typically comprise at least one degree of conjugation.

“Fluorescent” refers to a molecule that is capable of absorbing light of a particular frequency and emitting light of a different frequency. Fluorescence is well-known to those of ordinary skill in the art.

“Colored” refers to a molecule that absorbs light within the colored spectrum red, yellow, blue and the like).

“FRET” refers to Förster resonance energy transfer refers to a physical interaction whereby energy from the excitation of one moiety (e.g., a first chromophore or “donor”) is transferred to an adjacent moiety (e.g., a second chromophore or “acceptor”). “FRET” is sometimes also used interchangeably with fluorescence resonance energy transfer (i.e., when each chromophore is a fluorescent moiety). Generally. FRET requires that (1) the excitation or absorption spectrum of the acceptor chromophore overlaps with the emission spectrum of the donor chromophore; (2) the transition dipole moments of the acceptor and donor chromophores are substantially parallel (i.e., at about 0° or 180°); and (3) the acceptor and donor chromophores share a spatial proximity (i.e., close to each other). The transfer of energy from the donor to the acceptor occurs through non-radiative dipole-dipole coupling and the distance between the donor chromophore and acceptor chromophore is generally much less than the wavelength(s) of light.

“Donor” or “donor chromophore” refers to a chromophore (e.g., a fluorophore) that is or can be induced into an excited electronic state and may transfer its excitation energy to a nearby acceptor chromophore in a non-radiative fashion through long-range dipole-dipole interactions. Without wishing to be bound by theory, it is thought that the energy transfer occurs because the oscillating dipoles of the respective chromophores have similar resonance frequencies. A donor and acceptor that have these similar resonance frequencies are referred to as a “donor-acceptor pair(s),” which is used interchangeably with “FRET moieties” or “FRET dyes.”

“Acceptor” or “acceptor chromophore” refers to a chromophore e.g., a fluorophore) to which excitation energy from a donor chromophore is transferred via a non-radiative transfer through long-range dipole-dipole interaction.

“Stoke's shift” refers to a difference between positions (e.g., wavelengths) of the band maxima of absorption and emission spectra of an electronic transition (e.g., from excited state to non-excited state, or vice versa). In some embodiments, the compounds have a Stoke's shift greater than 25 nm, greater than 30 mm, greater than 35 nm, greater than 40 nm, greater than 45 nm, greater than 50 nm, greater than 55 nm, greater than 60 nm, greater than 65 nm, greater than 70 nm, greater than 75 nm, greater than 80 nm, greater than 85 nm, greater than 90 nm, greater than 95 nm, greater than 100 nm, greater than 110 inn, greater than 120 nm, greater than 130 nm, greater than 140 nm, greater than 150 nm, greater than 160 nm, greater than 170 nm, greater than 180 nm, greater than 190 nm, or greater than 200 nm.

A “linker” refers to a contiguous chain of at least one atom, such as carbon, oxygen, nitrogen, sulfur, phosphorous and combinations thereof, which connects a portion of a molecule to another portion of the same molecule or to a different molecule, moiety or solid support (e.g., microparticle). Linkers may connect the molecule via a covalent bond or other means, such as ionic or hydrogen bond interactions.

As used herein, “nucleic acid” or “nucleic acid molecule” or “polynucleotide” refers to any of deoxyribonucleic acid (DNA) or ribonucleic acid (RNA), including oligonucleotides.

The nucleic acid can represent a coding strand or its complement. Nucleic acids may be composed of monomers that are naturally occurring nucleotides (such as deoxyribonucleotides and ribonucleotides), analogs of naturally occurring nucleotides e.g., α-enantiomeric forms of naturally-occurring, nucleotides), or a combination of both. Nucleotides in a nucleic acid sequence are named according to standard IUPAC convention. Specifically, “A” is Adenine, “C” is Cytosine, “G” is Guanine, “T” is Thymine, “U” is Uracil, which refer to the following structures:

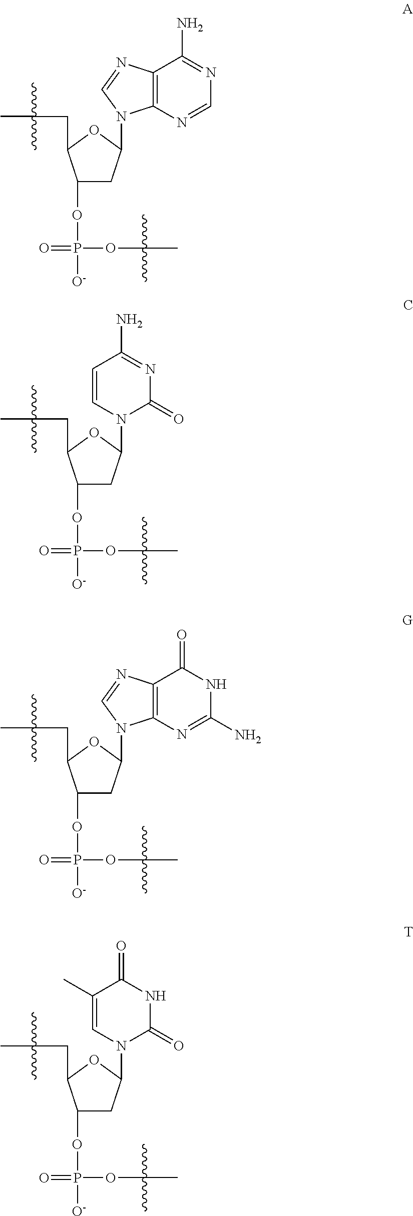 [SMILES: *CC1OC(n2cnc3c(N)ncnc32)CC1OP(=O)([O-])O* ; *CC1OC(n2ccc(N)nc2=O)CC1OP(=O)([O-])O* ; *CC1OC(n2cnc3c(=O)[nH]c(N)nc32)CC1OP(=O)([O-])O* ; *CC1OC(n2cc(C)c(=O)[nH]c2=O)CC1OP(=O)([O-])O*]

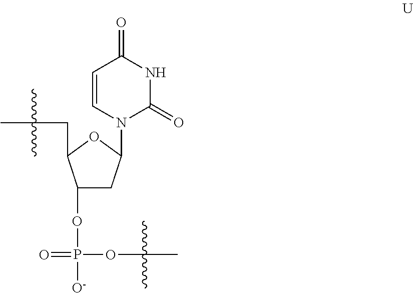 [SMILES: *CC1OC(n2ccc(=O)[nH]c2=O)CC1OP(=O)([O-])O*]

A sequence of a polynucleotide refers to the order in which nucleotides are arranged in a polynucleotide.

Analogs of naturally occurring nucleotides, also referred to as modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. In various embodiments, modified internucleotide linkages are used. Modified internucleotide linkages are well known in the art and include methylphosphonates, phosphorothioates, phosphorodithionates, phosphoroamidites and phosphate ester linkages. Nucleic acid molecules can be either single stranded or double stranded.

Analogs of naturally occurring nucleotides further include locked nucleic acids (UNA) also referred to as bridged nucleic acids (BNA). LNA contain a T-oxygen, 4′-carbon methylene bridge that clocks' the 3′-endo conformation, thereby restricting flexibility of the ribofuranose ring.

The term “hybridization” as used herein refers to any process by which a first strand of nucleic acid binds with a second strand of nucleic acid through base pairing. Hybridization can occur between fully complementary nucleic acid strands or between “substantially complementary” nucleic acid strands that contain minor regions of mismatch. Although hybridization is discussed herein generally with reference to duplex structures (i.e., structures of two nucleic acids) for ease of understanding, one of ordinary skill in the art would understand that the embodiments below also encompass configurations in which the nucleic acids described form triplex structures (i.e., structures of three nucleic acids). For example, two probes may hybridize with a single target sequence, or two detectable probes may hybridize with the same area of a capture probe.

As used herein, a probe is a polynucleotide that is “specific,” for a target sequence if, when used under sufficiently stringent conditions, the probe hybridizes primarily only to the target nucleic acid. Typically, a probe is specific for a target sequence if the probe-target duplex (or triplex) stability is greater than the stability of a duplex (or triplex) formed between the probe and any other sequence found in the sample. One of skill in the art will recognize that various factors, such as salt conditions as well as base composition of the probe and the location of the mismatches, will affect the specificity of the probe, and that routine experimental confirmation of the probe specificity will be needed in most cases. Hybridization conditions can be chosen under which the probe can form stable duplexes (or triplexes) only with a target sequence. Thus, the use of target-specific probes under suitably stringent conditions enables the specific amplification of those target sequences that contain the target probe binding sites. The use of sequence-specific conditions enables the specific binding of the probes to the target sequences that contain the exactly complementary probe binding sites.

Conditions under which only fully complementary nucleic acid strands will hybridize are referred to as “stringent hybridization conditions” or “sequence-specific hybridization conditions”. The term “stringent” as used herein refers to hybridization conditions that are commonly understood in the art to define the conditions of the hybridization procedure. Stringency conditions can be low, high or medium, as those terms are commonly known in the art and well recognized by one of ordinary skill. In various embodiments, stringent conditions can include, for example, highly stringent conditions, and/or moderately stringent (i.e., medium stringency) conditions.

Stable duplexes (or triplexes) of substantially complementary sequences can be achieved under less stringent hybridization conditions; the degree of mismatch tolerated can be controlled by suitable adjustment of the hybridization conditions. Those skilled in the art of nucleic acid technology can determine duplex (or triplex) stability empirically considering a number of variables including, for example, the length and base pair composition of the oligonucleotides, ionic strength, and incidence of mismatched base pairs, following the guidance provided by the art.

As used herein, “complementary” refers to a nucleic acid molecule that can form hydrogen bond(s) with another nucleic acid molecule by either traditional Watson-Crick base pairing or other non-traditional types of pairing (e.g., Hoogsteen or reversed Hoogsteen hydrogen bonding) between complementary nucleosides or nucleotides.

It is understood in the art that a nucleic acid molecule need not be 100% complementary to a target nucleotide sequence to be specifically hybridizable. That is two or more nucleic acid molecules may be less than fully complementary and is indicated by a percentage of contiguous residues in a nucleic acid molecule that can form hydrogen bonds with a second nucleic acid molecule. For example, if a first nucleic acid molecule has 10 nucleotides and a second nucleic acid molecule has 10 nucleotides, then base pairing 5 of 5, 6, 7, 8, 9, or 10 nucleotides between the first and second nucleic acid molecules represents 50%, 60%, 70%, 80%, 90%, and 100% complementarity, respectively. “Perfectly” or “fully” complementary nucleic acid molecules means those in which all the contiguous residues of a first nucleic acid molecule will hydrogen bond with the same number of contiguous residues in a second nucleic acid molecule, wherein the nucleic acid molecules either both have the same number of nucleotides (i.e., have the same length) or the two molecules have different lengths.

The term “hybridization complex” as used herein refers to a complex formed between two nucleotide sequences by virtue of the formation of hydrogen bonds between complementary G and C bases and between complementary A and T bases; these hydrogen bonds may be further stabilized by base stacking interactions. The two complementary nucleotide sequences hydrogen bond in an antiparallel configuration. A hybridization complex may be formed in solution (e.g., Cot or Rot analysis) or between one nucleotide sequence present in solution and another nucleotide sequence immobilized on a solid support (e.g., paper, membranes, filters, chips, pins or glass slides, or any other appropriate substrate to which cells and/or nucleic acids have been fixed).

A “reactive group” is a moiety capable of reacting with a second reactive groups (e.g., a “complementary reactive group”) to form one or more covalent bonds, for example by a displacement, oxidation, reduction, addition or cycloaddition reaction. Exemplary reactive groups include for example, nucleophiles, electrophiles, dienes, dienophiles, aldehyde, oxime, hydrazone, alkyne, amine, azide, acylazide, acylhalide, nitrile, nitrone, sulfhydryl, disulfide, sulfonyl halide, isothiocyanate, imidoester, activated ester, ketone, α,β-unsaturated carbonyl, alkene, maleimide, epoxide, aziridine, tetrazine, tetrazole, phosphine, biotin, thiirane and the like.

The terms “visible” and “visually detectable” are used herein, to refer to substances that are observable by visual inspection, without prior illumination, or chemical or enzymatic activation. Such visually detectable substances absorb and emit light in a region of the spectrum ranging from about 300 to about 900 nm. Preferably, such substances are intensely colored, preferably having a molar extinction coefficient of at least about 40,000, more preferably at least about 50,000, still more preferably at least about 60,000, yet still more preferably at least about 70,000, and most preferably at least about 80,000 M-1 cm-1. The compounds of the invention may be detected by observation with the naked eye, or with the aid of an optically based detection device, including, without limitation, absorption spectrophotometers, transmission light microscopes, digital cameras and scanners. Visually detectable substances are not limited to those emit and/or absorb light in the visible spectrum. Substances which emit and/or absorb light in the ultraviolet (UV) region (about 10 nm to about 400 nm), infrared (IR) region (about 700 nm to about 1 mm), and substances emitting and/or absorbing in other regions of the electromagnetic spectrum are also included with the scope of “visually detectable” substances.

For purposes of embodiments of the invention, the term “photostable visible dye” refers to a chemical moiety that is visually detectable, as defined hereinabove, and is not significantly altered or decomposed upon exposure to light. Preferably, the photostable visible dye does not exhibit significant bleaching or decomposition after being exposed to light for at least one hour. More preferably, the visible dye is stable after exposure to light for at least 12 hours, still more preferably at least 24 hours, still yet more preferably at least one week, and most preferably at least one month. Nonlimiting examples of photostable visible dyes suitable for use in the compounds and methods of the invention include azo dyes, thioindigo dyes, quinacridone pigments, dioxazine, phthalocyanine, perinone, diketopyrrolopyrrole, quinophthalone, and truarycarbonium.

As used herein, the term “perylene derivative” is intended to include any substituted perylene that is visually detectable. However, the term is not intended to include perylene itself. The terms “anthracene derivative”, “naphthalene derivative”, and “pyrene derivative” are used analogously. In some preferred embodiments, a derivative (e.g., perylene, pyrene, anthracene or naphthalene derivative) is an imide, bisimide or hydrazamimide derivative of perylene, anthracene, naphthalene, or pyrene.

The visually detectable molecules of various embodiments of the invention are useful for a wide variety of analytical applications, such as biochemical and biomedical applications, in which there is a need to determine the presence, location, or quantity of a particular analyte (e.g., biomolecule). In another aspect, therefore, the invention provides a method for visually detecting a biomolecule, comprising: (a) providing a biological system with a visually detectable biomolecule comprising the compound of structure (I) linked to a biomolecule; and (b) detecting the biomolecule by its visible properties. For purposes of the invention, the phrase “detecting the biomolecule by its visible properties” means that the biomolecule, without illumination or chemical or enzymatic activation, is observed with the naked eye, or with the aid of a optically based detection device, including, without limitation, absorption spectrophotometers, transmission light microscopes, digital cameras and scanners. A densitometer may be used to quantify the amount of visually detectable biomolecule present. For example, the relative quantity of the biomolecule in two samples can be determined by measuring relative optical density. If the stoichiometry of dye molecules per biomolecule is known, and the extinction coefficient of the dye molecule is known, then the absolute concentration of the biomolecule can also be determined from a measurement of optical density. As used herein, the term “biological system” is used to refer to any solution or mixture comprising one or more biomolecules in addition to the visually detectable biomolecule. Examples of such biological systems include cells, cell extracts, tissue samples, electrophoretic gels, assay mixtures, and hybridization reaction mixtures.

“Solid support” refers to any solid substrate known in the art for solid-phase support of molecules, for example a “microparticle” refers to any of a number of small particles useful for attachment to compounds of the invention, including, for example, glass beads, magnetic beads, polymeric beads, nonpolymeric heads, and the like. In certain embodiments, a microparticle comprises polystyrene beads.

A “solid support reside” refers to the functional group remaining attached to a molecule when the molecule is cleaved from the solid support. Solid support residues are known in the art and can be easily derived based on the structure of the solid support and the group linking the molecule thereto.

The word “target,” as used herein, refers to an analyte of interest that is to be detected. A “targeting moiety” is a moiety that selectively binds or associates with a particular target, such as an analyte molecule. “Selectively” binding or associating means a targeting moiety preferentially associates or binds with the desired target relative to other targets. In some embodiments the compounds disclosed herein include linkages to targeting moieties for the purpose of selectively binding or associating the compound with an analyte of interest (i.e., the target of the targeting moiety), thus allowing detection of the analyte. Exemplary targeting moieties include, for example, antibodies, antigens, nucleic acid sequences, enzymes, proteins, cell surface receptor antagonists, and the like. In some embodiments, the targeting moiety is a moiety, such as an antibody, that selectively binds or associates with a target feature on or in a cell, for example a target feature on a cell membrane or other cellular structure, thus allowing for detection of cells of interest. Small molecules that selectively bind or associate with a desired analyte are also contemplated as targeting moieties in certain embodiments. One of skill in the art will understand other analytes, and the corresponding targeting moiety, that will be useful in various embodiments.

“Base pairing moiety” refers to a heterocyclic moiety capable of hybridizing with a complementary heterocyclic moiety via hydrogen bonds (e.g., Watson-Crick base pairing). Base pairing moieties include natural and unnatural bases. Non-limiting examples of base pairing moieties are RNA and DNA bases such adenosine, guanosine, thymidine, cytosine and uridine and analogues thereof.

As used herein, a “capture probe” is a single stranded DNA molecule that is bound e.g., covalently bound or hybridized) to a solid support or a targeting moiety. Generally, the single stranded DNA molecule comprises a repeating nucleotide sequence that is complementary to the nucleotide sequence of a detectable probe or a branched linker, such that multiple detectable probes or branched linkers are capable of hybridizing to a single capture probe.

A “detectable probe” comprises a nucleotide sequence that is bound to at least one chromophore.

As used herein, a “branched linker” refers to a polymer backbone to which a plurality of polynucleotide are bound such that the polynucleotides extend away from the polymer backbone. For example, a branched linker may be a polyalkynes conjugated to a plurality of DNA molecules. The plurality of polynucleotide comprises at least one polynucleotide having a first sequence. The first sequence is complementary to a repeating nucleotide sequence of a capture probe. Additionally, the plurality of polynucleotide comprises a plurality polynucleotides having a second sequence, which is complementary to the nucleotide sequence of a detectable probe.

Embodiments of the invention disclosed herein are also meant to encompass all compounds of Structure (I), (II) or (III) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as 2H, 3H, 11C, 13C, 14C, 13N, 15N, 15O, 17O, 18O, 31P, 32P, 35S, 18F, 36Cl, 123I, and 125I, respectively.

Isotopically-labeled compounds of Structure (I) or (II) can generally be prepared by conventional techniques known to those skilled in the art or by processes analogous to those described below and in the following Examples using an appropriate isotopically-labeled reagent in place of the non-labeled reagent previously employed.

“Stable compound” and “stable structure” are meant to indicate a compound that is sufficiently robust to survive isolation to a useful degree of purity from a reaction mixture, and formulation into an efficacious therapeutic agent.

“Optional” or “optionally” means that the subsequently described event or circumstances may or may not occur, and that the description includes instances where said event or circumstance occurs and instances in which it does not. For example, “optionally substituted alkyl” means that the alkyl group may or may not be substituted and that the description includes both substituted alkyl groups and alkyl groups having no substitution.

“Salt” includes both acid and base addition salts.

“Acid addition salt” refers to those salts that are formed with inorganic acids such as hydrochloric acid, hydrobromic acid, sulfuric acid, nitric acid, phosphoric acid and the like, and organic acids such as acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecyl sulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Base addition salt” refers to those salts that are prepared from addition of an inorganic base or an organic base to the free acid. Salts derived from inorganic bases include, for example, sodium, potassium, lithium, ammonium, calcium, magnesium, iron, zinc, copper, manganese, aluminum salts and the like. Salts derived from organic bases include, for example, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanol amine, deanol, 2 dimethylaminoethanol, 2 diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly preferred organic bases are isopropylamine, diethylamine, ethanolamine, trimethylamine, dicyclohexylamine, choline and caffeine.

Crystallizations may produce a solvate of the compounds described herein. Embodiments of the present invention include all solvates of the described compounds. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compounds of the invention may be true solvates, while in other cases the compounds of the invention may merely retain adventitious water or another solvent or be a mixture of water plus some adventitious solvent.

Embodiments of the compounds of the invention (e.g., compounds of structure I, IF, or III), or their salts, tautomers or solvates may contain one or more asymmetric centers and may thus give rise to enantiomers, diastereomers, and other stereoisomeric forms that may be defined, in terms of absolute stereochemistry, as (R) or (S) or, as (D) or (L) for amino acids. Embodiments of the present invention are meant to include all such possible isomers, as well as their racemic and optically pure forms. Optically active (+) and (−), (R) and (S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds. Various tautomeric forms of the compounds are easily derivable by those of ordinary skill in the art.

The chemical naming protocol and structure diagrams used herein are a modified form of the I.U.P.A.C. nomenclature system, using the ACD/Name Version 9.07 software program and/or ChemDraw Ultra Version 11.0 software naming program (CambridgeSoft). Common names familiar to one of ordinary skill in the art are also used.

As noted above, in one embodiment of the present invention, compounds useful as probes in various analytical methods are provided. In general terms, embodiments of the present application are directed to compounds that can be used as capture or detectable probes, as well as compositions comprising the same.

In some embodiments, compounds of the disclosure have the following structure (I):

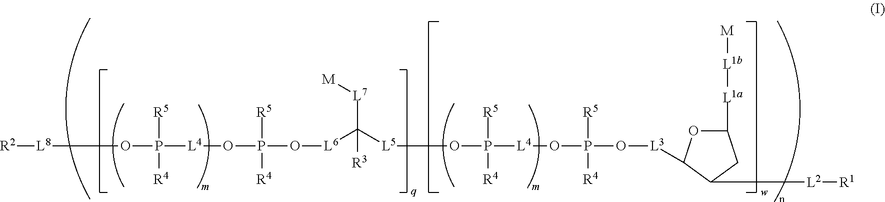

or a stereoisomer, salt or tautomer thereof, wherein:
M is, at each occurrence, independently the same or different chromophore;
L^{1a} is, at each occurrence, independently a heteroarylene linker;
L² and L⁸ are independently optional linkers;
L^{1b}, L³, L⁵, L⁶, and L⁷ are, at each occurrence, independently optional alkylene, alkenylene, alkynylene, heteroalkylene, heteroalkenylene, or heteroalkynylene linkers;
L⁴ is, at each occurrence, independently an alkylene, alkenylene, alkynylene, heteroalkylene, heteroalkenylene, or heteroalkynylene linker;
R¹ and R² each independently H, OH, SH, alkyl, alkoxy, alkylether, heteroalkyl, —OP(—Rₐ)(R_{b})R_{c} or a moiety comprising a polynucleotide, provided at least one of R¹ and R² is a moiety comprising a polynucleotide;
R³ is, at each occurrence, independently H, alkyl, or alkoxy;
R³ is, at each occurrence, independently OH, SH, O⁻, S⁻, OR_{d}, or SR_{d};
R⁵ is, at each occurrence, independently oxo, thioxo, or absent;
Rₐ is O or S;
R_{b} is OH, SH, O⁻, S⁻, OR_{d}, or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether, or thiophosphoalkylether;
R_{d} is a counter ion;
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of one or greater; and
q and w are, at each occurrence, independently 0 or 1, provided at least one occurrence of either q or w is 1.

In some embodiments, M is, at each occurrence, independently the same or different donor chromophores. In other embodiments, M is, at each occurrence, independently the same or different acceptor chromophores. In certain embodiments, M is the same donor chromophore at each occurrence. In other embodiments, M is the same acceptor chromophore at each occurrence.

In embodiments, M is, at each occurrence, independently the same or different fluorophore. In some embodiments, M is, at each occurrence, independently the same or a different donor fluorophore. In other embodiments, M is, at each occurrence, independently the same or different acceptor fluorophore. In certain embodiments, M is the same donor fluorophore at each occurrence. In other embodiments, NI is the same acceptor fluorophore at each occurrence.

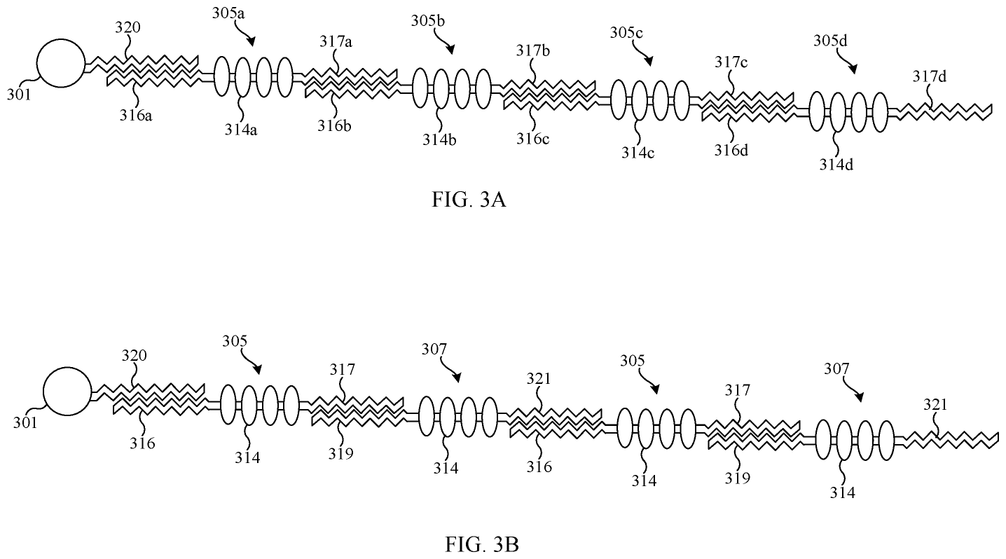

In some embodiments, R¹ and R² are each a moiety comprising a polynucleotide. In some such embodiments, the polynucleotide of R¹ is complementary to the polynucleotide of R² such that, the polynucleotide of R¹ of a first compound is capable of hybridizing to the polynucleotide of R² of a second compound, and so forth. An example of this is illustrated in FIG. 3A. A targeting moiety 301 (in this case, a coated bead) is bound to a first polynucleotide 320. Probes 305a, 305h, 305c, 305d are independently compounds of structure (I). Each of the probes independently comprises a polymer 314a, 314h, 314c, 314d arranged between a second polynucleotide 316a, 316h, 316c, 316d and a third polynucleotide 317a, 317b, 317c, 317d. As shown in FIG. 3A, the second polynucleotide 316b of probe 305b hybridizes to the third polynucleotide 317a of probe 305a. In some embodiments, a triplex structure is formed by the polynucleotides of three probes.

In embodiments, a second sequence has at least 92% complementarity to a third sequence. In some embodiments, a second sequence has at least 95% complementarity to a third sequence. In some embodiments, a second sequence has at least 96% complementarity to a third sequence. In some embodiments, a second sequence has at least 97% complementarity to a third sequence. In some embodiments, a second sequence has at least 98% complementarity to a third sequence. In some embodiments, a second sequence has at least 99% complementarity to a third sequence.

Additionally, in some embodiments, the plurality of probes comprises two or more probes 305, 307 that alternate or repeat in a pattern (as shown in FIG. 3B). In some such embodiments, each of the first probes 305 independently comprises a polymer 314 arranged between a second polynucleotide 316 and a third polynucleotide 317, and each of the second probes 307 comprises a polymer 314 arranged between a fourth polynucleotide 319 and a fifth polynucleotide 321. The third polynucleotide 317 hybridizes to the fourth polynucleotide 319 and the fifth polynucleotide 321 hybridizes to the second polynucleotide. In some embodiments, a triplex structure is formed by the polynucleotides of three probes.

In some embodiments, each of the first polymers comprise a plurality of chromophores. In embodiments, the second polynucleotide, third polynucleotide, or both has a length ranging from 10 nucleotides to 40 nucleotides. In some embodiments, the second polynucleotide, third polynucleotide, or both has a length ranging from 10 nucleotides to 30 nucleotides. In some embodiments, the second polynucleotide, third polynucleotide, or both has a length ranging from 15 nucleotides to 25 nucleotides.

One of the second polynucleotides is hybridized to the first polynucleotide. In various embodiments, the first polynucleotide having a first sequence that has at least 90% complementarity to a second sequence. In some embodiments, the first sequence has at least 92% complementarity to a second sequence. In some embodiments, a second sequence has at least 95% complementarity to the first sequence. In some embodiments, a second sequence has at least 96% complementarity to the first sequence. In some embodiments, a second sequence has at least 97% complementarity to the first sequence. In some embodiments, a second sequence has at least 98% complementarity to the first sequence. In some embodiments, a second sequence has at least 99% complementarity to the first sequence. In other embodiments, a targeting moiety is covalently bound to the capture probe.

In other embodiments, compounds of the present disclosure have the following structure (II):

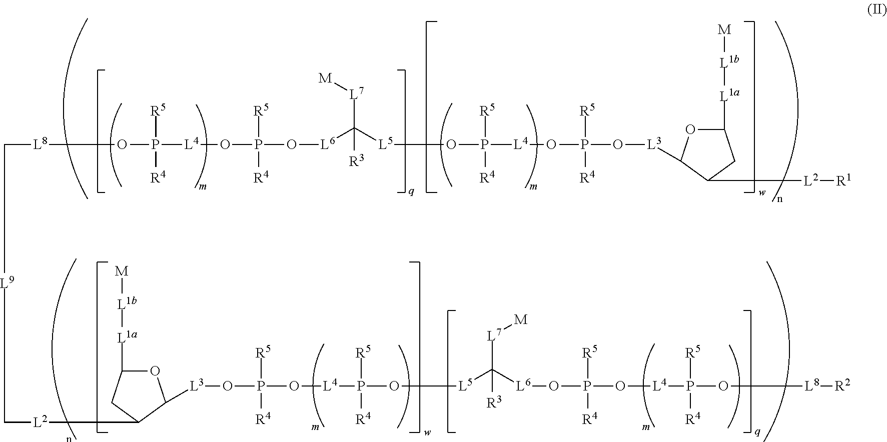

or a stereoisomer, salt or tautomer thereof, wherein:
M is, at each occurrence, independently a chromophore;
L^{1a} is, at each occurrence, independently a heteroarylene linker;
L² and L⁸ are independently optional linkers:
L^{1b}, L³, L⁵, L⁶, and L⁷ are, at each occurrence, independently optional alkylene, alkenylene, alkynylene, heteroalkylene, heteroalkenylene, or heteroalkynylene linkers;
L⁴ is, at each occurrence, independently an alkylene, alkenylene, alkynylene, heteroalkylene, heteroalkenylene, or heteroalkynylene linker;
L⁹ is a linker comprising a polynucleotide;
R¹ and R² are each independently H, OH, SH, alkyl, alkoxy, alkylether, heteroalkyl, or —OP(═Rₐ)(R_{b})R_{c};
R³ is, at each occurrence, independently H, alkyl, or alkoxy;
R⁴ is, at each occurrence, independently OH, SH, O⁻, S⁻, OR_{d}, or SR_{d};
R⁵ is, at each occurrence, independently oxo, thioxo, or absent;
Rₐ is O or S;
R_{b} is OH, SH, O⁻, S⁻, OR_{d}, or SR_{d};
R_{d} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether, or thiophosphoalkylether;
R_{d} is a counter ion;
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of one or greater; and
q and w are, at each occurrence, independently 0 or 1, provided at least one occurrence of either q or w is 1.

In some embodiments, M is, at each occurrence, independently the same or a different donor chromophore. In other embodiments, M is, at each occurrence, independently the same or different acceptor chromophores. In certain embodiments, M is the same donor chromophore at each occurrence. In other embodiments, M is the same acceptor chromophore at each occurrence.

In embodiments, M is, at each occurrence, independently the same or a different fluorophore. In some embodiments, M is, at each occurrence, independently the same or a different donor fluorophore. In other embodiments, M is, at each occurrence, independently the same or a different acceptor fluorophore. In certain embodiments, M is the same donor fluorophores at each occurrence. In other embodiments, M is the same acceptor fluorophore at each occurrence.

In some embodiments, compounds of the present disclosure have the following structure (IIa) or (IIb):

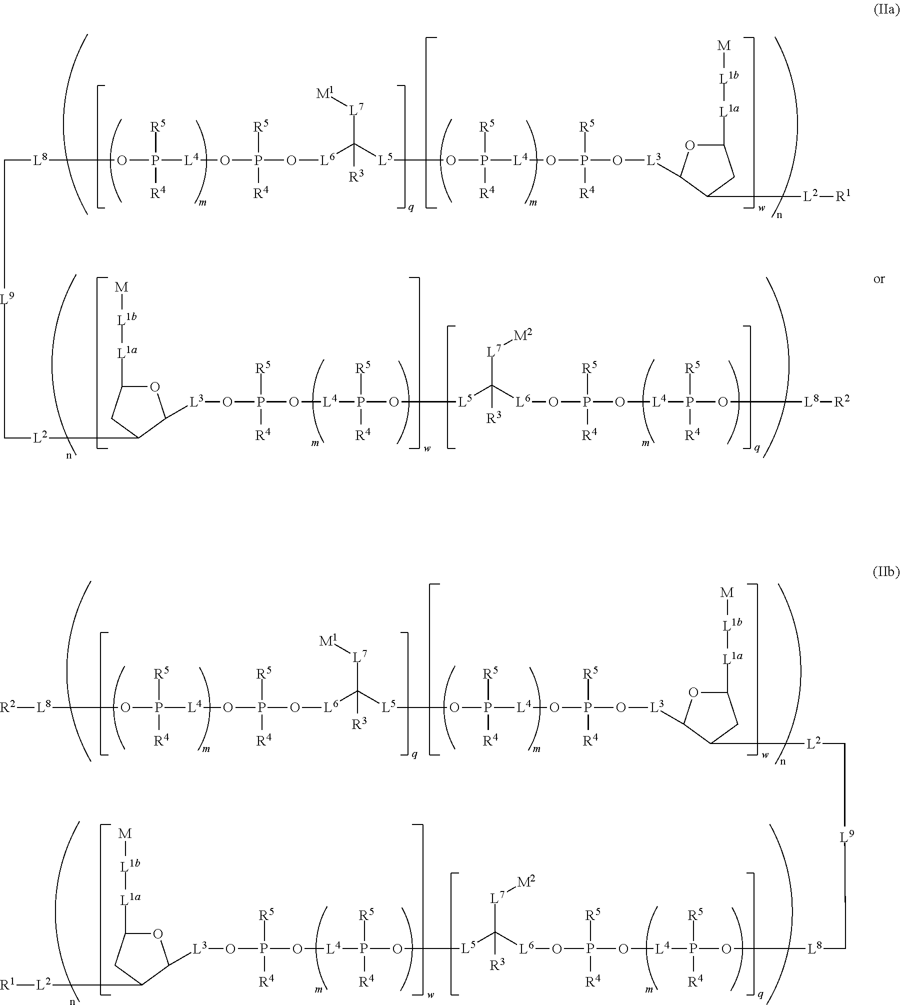

or a stereoisomer, salt or tautomer thereof, wherein:
M¹ is, at each occurrence, independently the same or different donor chromophores;
M² is, at each occurrence, independently the same or different acceptor chromophores,
L^{1a} is at each occurrence, independently a heteroarylene linker;
L² and L⁸ are independently optional linkers:
L^{1b}, L³, L⁵, L⁶, and L⁷ are, at each occurrence, independently optional alkylene, alkenylene, alkynylene, heteroalkylene, heteroalkenylene, or heteroalkynylene linkers; is, at each occurrence, independently an alkylene, alkenylene, alkynylene, heteroalkylene, heteroalkenylene, or heteroalkynylene linker;
L⁹ is a linker comprising a polynucleotide;
R¹ and R² are each independently H, OH, SH, alkyl, alkoxy, alkylether, heteroalkyl, or —OP(═Rₐ)(R_{b})R_{c};
R³ is, at each occurrence, independently H, alkyl, or alkoxy;
R⁴ is, at each occurrence, independently OH, SH, O⁻, S⁻, OR_{d}, or SR_{d};
R⁵ is, at each occurrence, independently oxo, thioxo, or absent;
Rₐ is O or S;
R_{b} is OH, SH, O⁻, S⁻, OR_{d}, or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether, or thiophosphoalkylether;
R_{d} is a counter ion;
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of one or greater; and
q and w are, at each occurrence, independently 0 or 1, provided at least one occurrence of either q or w is 1.

In some embodiments, compounds of the present disclosure have the structure (IIa). In some embodiments, compounds of the present disclosure have the structure (IIb).

In some embodiments, M¹ is, at each occurrence, independently the same or different donor fluorophores. In other embodiments, M² is, at each occurrence, independently the same or different acceptor fluorophore. In certain embodiments, M¹ is the same donor fluorophore at each occurrence. In other embodiments, M² is the same acceptor fluorophore at each occurrence.

In embodiments, the polynucleotide of L⁹ has a length ranging from 10 nucleotides to 40 nucleotides. In some embodiments, the polynucleotide of L⁹ has a length ranging from 10 nucleotides to 30 nucleotides. In some embodiments, the polynucleotide of L⁹ has a length ranging from 1.5 nucleotides to 25 nucleotides.

In embodiments, compounds described herein having the structure (I), (II), (IIa), (IIb), or a combination thereof, are detectable probes. In some embodiments, a compound described herein having the structure (I) is a detectable probe. In some embodiments, a compound described herein having the structure (II) is a detectable probe. In some embodiments, a compound described herein having the structure (IIa) is a detectable probe. In some embodiments, a compound described herein having the structure (IIb) is a detectable probe.

Accordingly, in some embodiments, compounds of the disclosure have the following structure (III):

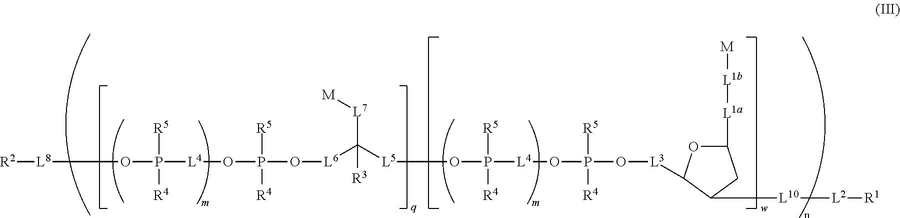

or a stereoisomer, salt or tautomer thereof, wherein:
M is, at each occurrence, independently the same or different chromophore;
L^{1a} is, at each occurrence, independently a heteroarylene linker;
L² and L⁸ are independently optional linkers;
L^{1b}, L³, L⁵, L⁶, and L⁷ are, at each occurrence, independently optional alkylene, alkenylene, alkenylene, heteroalkylene, heteroalkenylene, or heteroalkynylene linkers;
L⁴ is, at each occurrence, independently an alkylene, alkenylene, alkynylene, heteroalkylene, heteroalkenylene, or heteroalkynylene linker;
L¹⁰ is a linker comprising a polynucleotide;
R¹ and R² each independently H, OH, SH, alkyl, alkoxy, alkylether, heteroalkyl, —OP(═Rₐ)(R_{b})R_{c} or a moiety comprising a polynucleotide, provided at least one of R¹ and R² is a moiety comprising a polynucleotide;
R³ is, at each occurrence, independently H, alkyl, or alkoxy;
R⁴ is; at each occurrence, independently OH, SH, O⁻, S⁻, OR_{d}, or SR_{d};
R⁵ is, at each occurrence, independently oxo, thioxo, or absent;
Rₐ is O or S;
R_{b} is OH, SH, O⁻, S⁻, OR_{d}, or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether, or thiophosphoalkylether;
R_{d} is a counter ion:
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of two or greater; and
q and w are, at each occurrence, independently 0 or 1, provided at least one occurrence of either q or w is 1.

In some embodiments, M is, at each occurrence, independently the same or different donor chromophores. In other embodiments, M is, at each occurrence, independently the same or different acceptor chromophores. In certain embodiments, M is the same donor chromophore at each occurrence, in other embodiments, M is the same acceptor chromophore at each occurrence.

In embodiments. M is, at each occurrence, independently the same or different fluorophore. In some embodiments, M is, at each occurrence, independently the same or different donor fluorophores. In other embodiments, M is, at each occurrence, independently the same or different acceptor fluorophore. In certain embodiments, M is the same donor fluorophores at each occurrence. In other embodiments, M is the same acceptor fluorophore at each occurrence.

In embodiments, R¹ comprises a polynucleotide. In some embodiments, R² comprises a polynucleotide. In specific embodiments, R¹ comprises a targeting moiety bound to the polynucleotide. In specific embodiments, R² comprises a targeting moiety bound to the polynucleotide.

In embodiments, a compound of structure (III) is a capture probe.

The polynucleotides in any of structures (I), (II) or (III), when at a terminal portion of the compound may terminate in any acceptable group. For example, certain polynucleotides will terminate in either a hydroxyl or phosphate group. In other various embodiments, the polynucleotides will terminate in —OP(═Rₐ)(R_{b})R_{c}, wherein R_{c} is OL′ and Rₐ and R_{b} are as defined above. In some of those embodiments, L′ is a heteroalkylene linker to a solid support, a solid support residue or a nucleoside. In some embodiments, L′ comprises an alkylene oxide or phosphodiester moiety, or combinations thereof. In certain embodiments, L′ has the following structure:

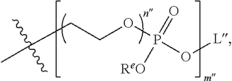

wherein:
m″ and n″ are independently an integer from 1 to 10;
R^{c} is H, an electron pair or a counter ion;
L″ is R^{c} or a direct bond or linkage to a solid support, a solid support residue or a nucleoside (e.g., deoxythymidine).

In some embodiments, the polynucleotides terminate in the following structure:

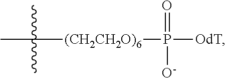

wherein dT is deoxythymidine:

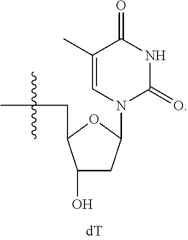 [SMILES: *CC1OC(n2cc(C)c(=O)[nH]c2=O)CC1O]

In embodiments, each occurrence of q is 0. In embodiments, at least one occurrence of q is 1. In embodiments, each occurrence of w is 0. In embodiments, at least one occurrence of w is 1. In embodiments, each occurrence of q is 0. In embodiments, at least one occurrence of L⁴ is heteroalkylene, or wherein each occurrence of L⁴ is heteroalkylene. In embodiments, the heteroalkylene comprises alkylene oxide. In other embodiments, the heteroalkylene comprises ethylene oxide. In some embodiments, L⁴ has the following structure:

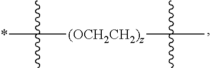

wherein:
Z is an integer from 1 to 100; and
* indicates a bond to the adjacent phosphorous atom.

In embodiments, z is an integer from 3 to 6 or an integer from 22 to 26. In embodiments, at least one occurrence of L⁴ is alkylene, or wherein each occurrence of L⁴ is alkylene. In various embodiments, at least one alkylene is ethylene, or wherein each alkylene is ethylene. In further embodiments, at least one occurrence of R³ is H, or wherein each occurrence of R³ is H. In some embodiments, L^{1a} is, at each occurrence independently an optionally substituted 5-7 membered heteroarylene linker. In embodiments, L^{1a} has one of the following structures:

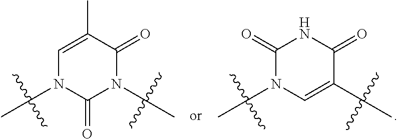 [SMILES: *n1cc(C)c(=O)n(*)c1=O ; *c1cn(*)c(=O)[nH]c1=O]

In embodiments, at least one occurrence of L³ is an alkylene linker, or wherein each occurrence of L³ is an alkylene linker. In some embodiments, at least one occurrence of L² and/or L⁸ is absent, or wherein each occurrence of L² and/or L⁸ is absent. In embodiments, at least one occurrence of L⁵ and/or L⁶ is alkylene, or wherein each occurrence of L⁵ and/or L⁶ is alkylene. In various embodiments, L^{1b}, at each occurrence, independently comprises an amide functional group or a triazolyl functional group.

In embodiments, R⁵ is, at each occurrence, independently OH, O⁻ or OR_{d}. In embodiments, wherein R⁴ is, at each occurrence, oxo.

In embodiments, at least one occurrence of L⁷ is an optionally substituted heteroalkylene linker, or wherein each occurrence of L⁷ is independently an optionally substituted heteroalkylene linker. In embodiments, L⁷ comprises an amide or a triazolyl functional group. In some embodiments, each occurrence of L⁷ has one of the following structures:

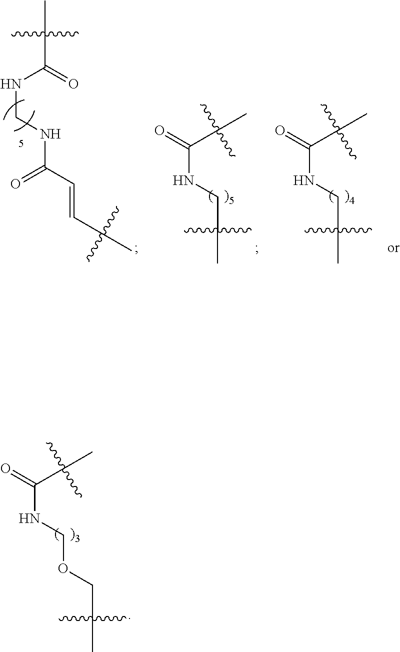

In embodiments, n is an integer from 1 to 100, or wherein n is an integer from 1 to 10, or from 2 to 10. For example, 1, 2, 3, 4, 5, 6, 7, 8, 9, or 10. In embodiments, m is an integer from 3 to 6, or wherein m is 3.

In embodiments, the fluorophore is, at each occurrence, independently a dimethylaminostilbene, quinacridone, fluorophenyl-dimethyl-BODIPY, his-fluorophenyl-BODIPY, acridine, terrylene, sexiphenyl, porphyrin, benzopyrene, (fluorophenyl-dimethyl-difluorobora-diaza-indacene)phenyl, (bis-fluorophenyl-difluorobora-diaza-indacene)phenyl, quaterphenyl, bi-benzothiazole, ter-benzothiazole, bi-naphthyl, bi-anthracyl, squaraine, squarylium, 9,10-ethynylanthracene, or ter-naphthyl moiety.

In embodiments, the fluorophore is, at each occurrence, independently pyrene, perylene, perylene monoimide, 5-FAM, or 6-FAM, or derivative thereof.

In further embodiments, the fluorophore is, at each occurrence, independently selected from Table 1.

TABLE 1
Exemplary Fluorophores
Structure

| | |
|---|---|
| 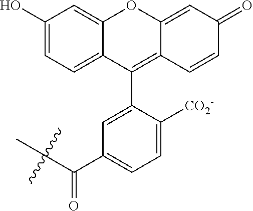 | F |

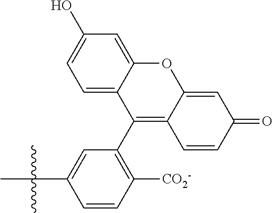

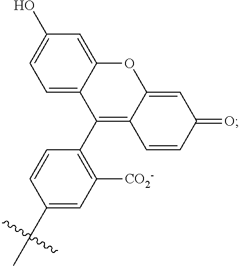

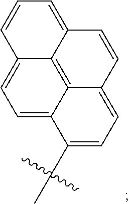 [SMILES: *c1ccc2ccc3cccc4ccc1c2c34]

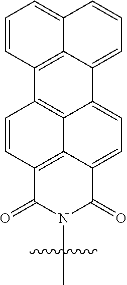 [SMILES: *N1C(=O)c2ccc3c4cccc5cccc(c6ccc(c2c36)C1=O)c54]

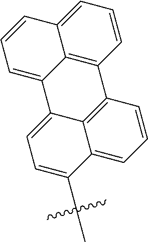 [SMILES: *c1ccc2c3cccc4cccc(c5cccc1c52)c43]

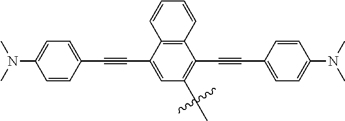

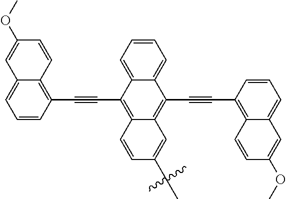 [SMILES: *c1ccc2c(C#Cc3cccc4cc(OC)ccc34)c3ccccc3c(C#Cc3cccc4cc(OC)ccc34)c2c1]

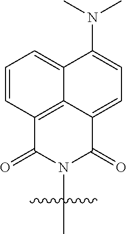 [SMILES: *N1C(=O)c2cccc3c(N(C)C)ccc(c23)C1=O]

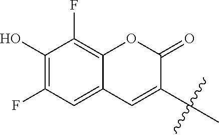 [SMILES: *c1cc2cc(F)c(O)c(F)c2oc1=O]

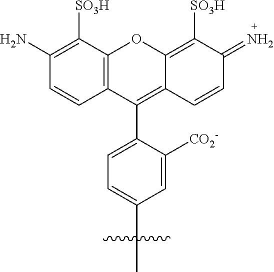

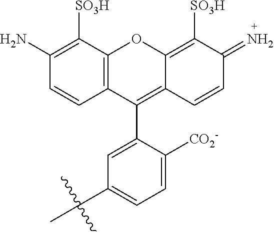

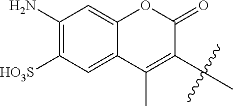 [SMILES: *c1c(C)c2cc(S(=O)(=O)O)c(N)cc2oc1=O]

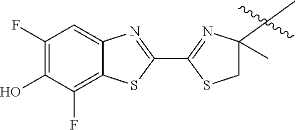 [SMILES: *C1(C)CSC(c2nc3cc(F)c(O)c(F)c3s2)=N1]

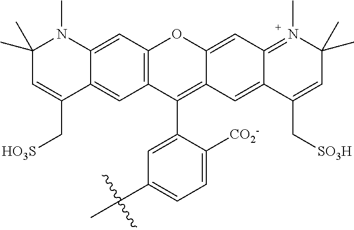

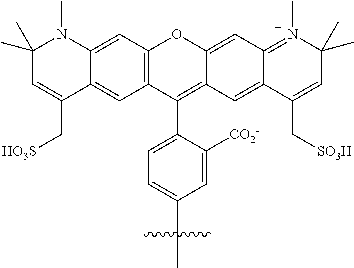

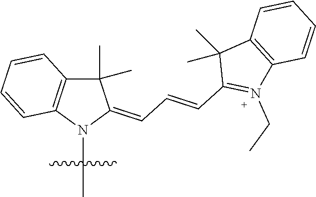

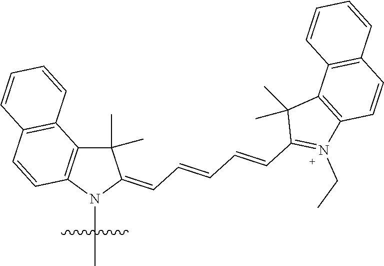

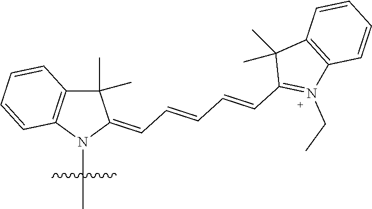

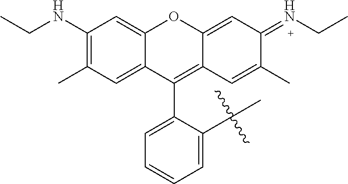

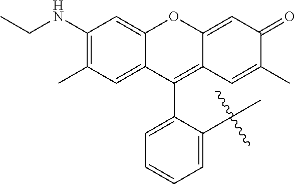 [SMILES: *c1ccccc1-c1c2cc(C)c(=O)cc-2oc2cc(NCC)c(C)cc12]

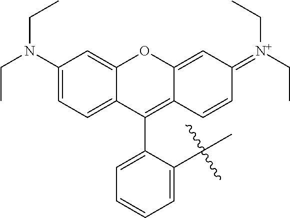 [SMILES: *c1ccccc1-c1c2ccc(=[N+](CC)CC)cc-2oc2cc(N(CC)CC)ccc12]

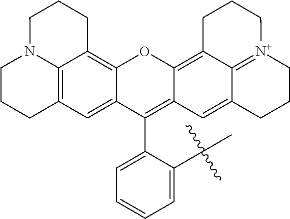 [SMILES: *c1ccccc1C1=c2cc3c4c(c2Oc2c1cc1c5c2CCCN5CCC1)CCC[N+]=4CCC3]

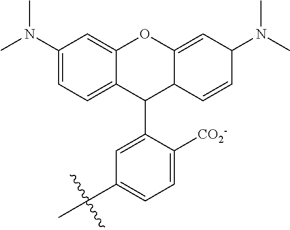

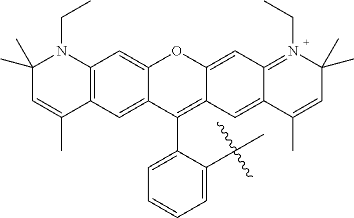

In particular embodiments, the fluorophore, at each occurrence, independently has one of the following structures:

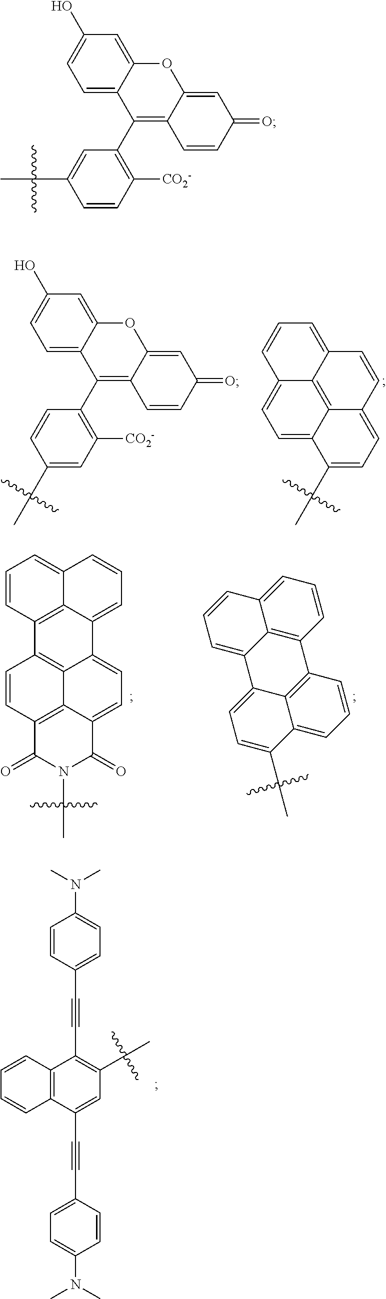

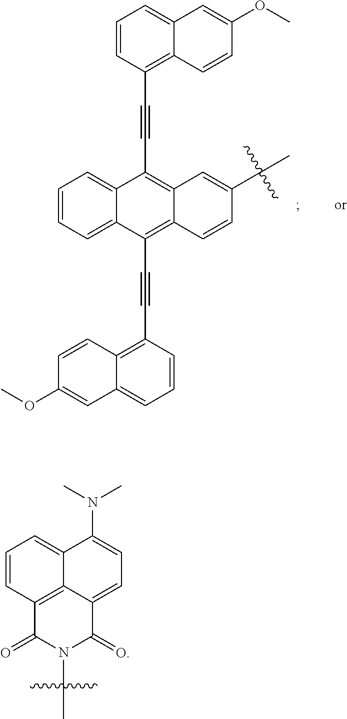

In embodiments, the fluorophore, at each occurrence, independently has one of the following structures:

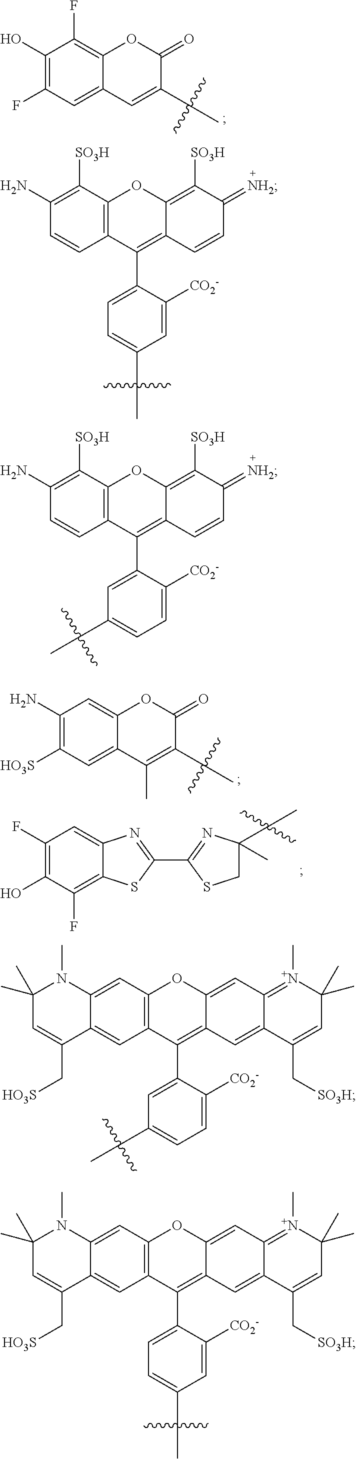

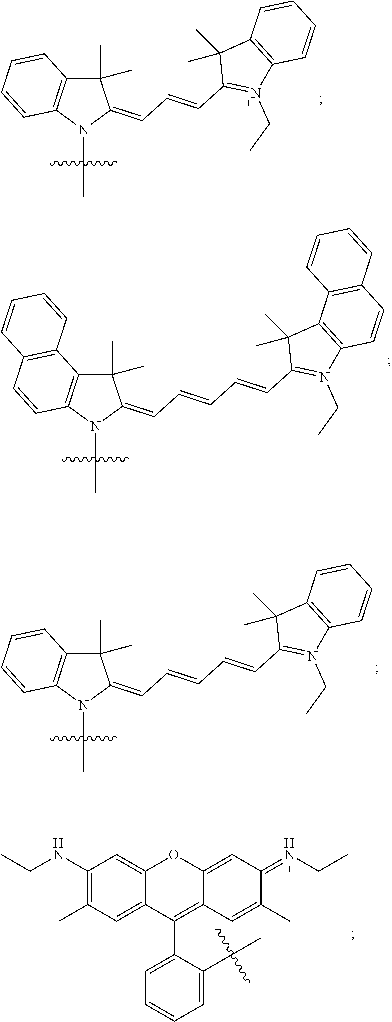

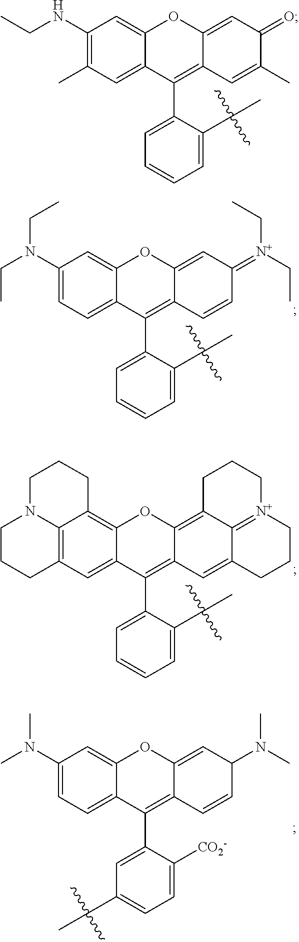

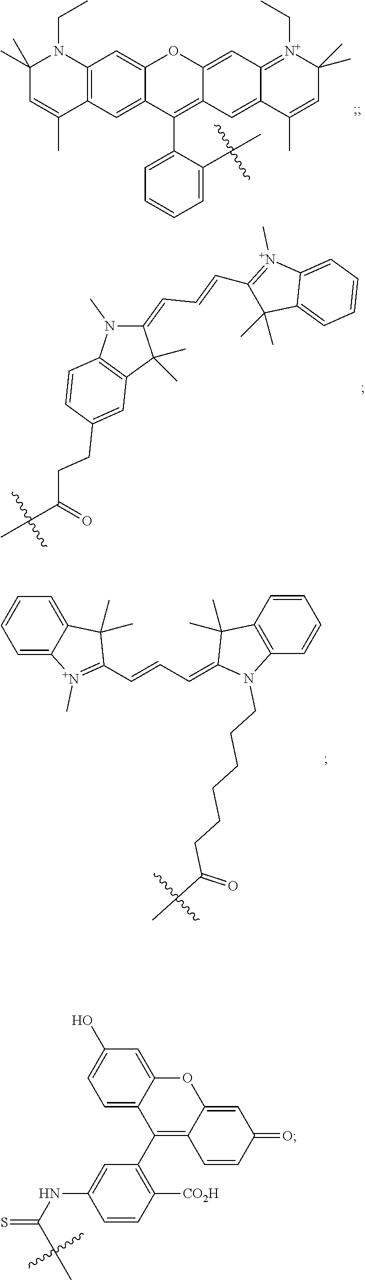

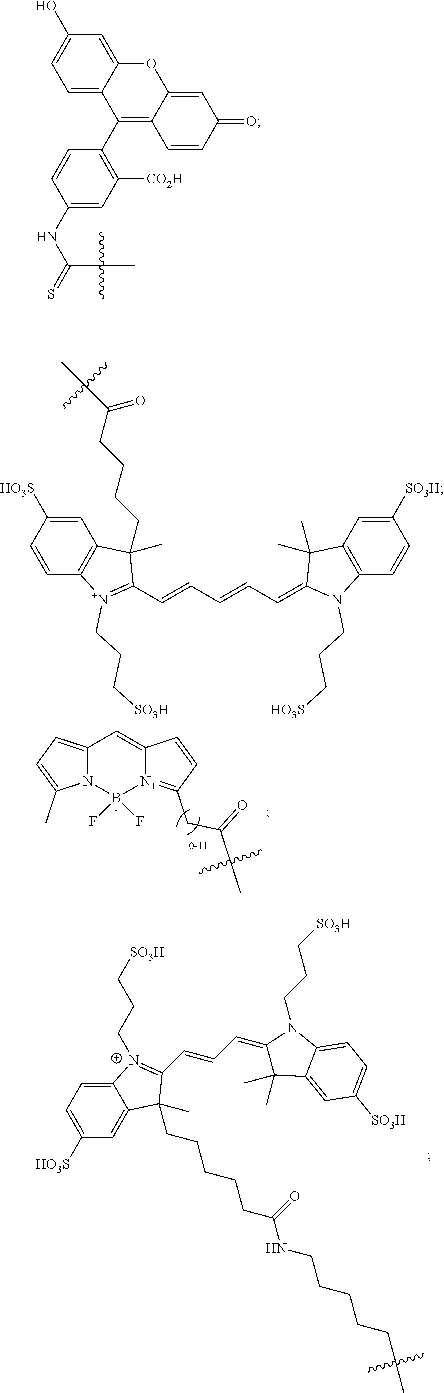

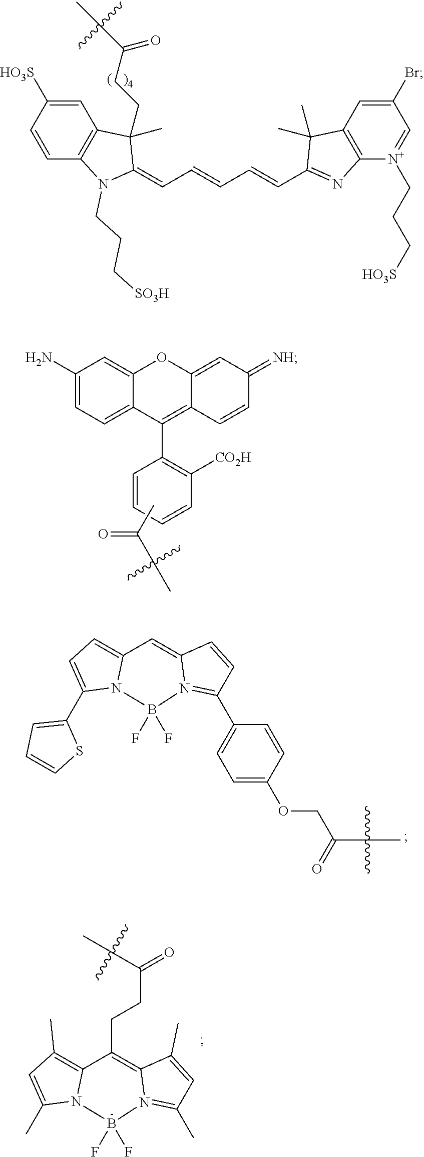

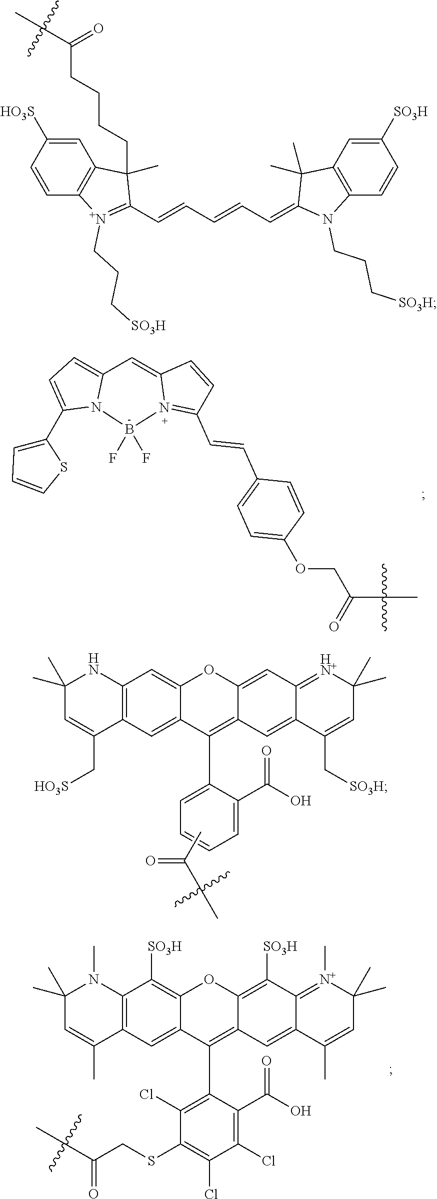

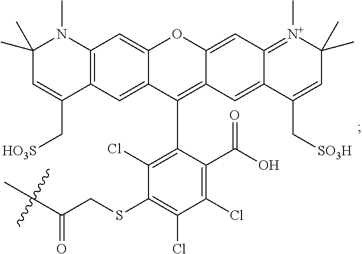

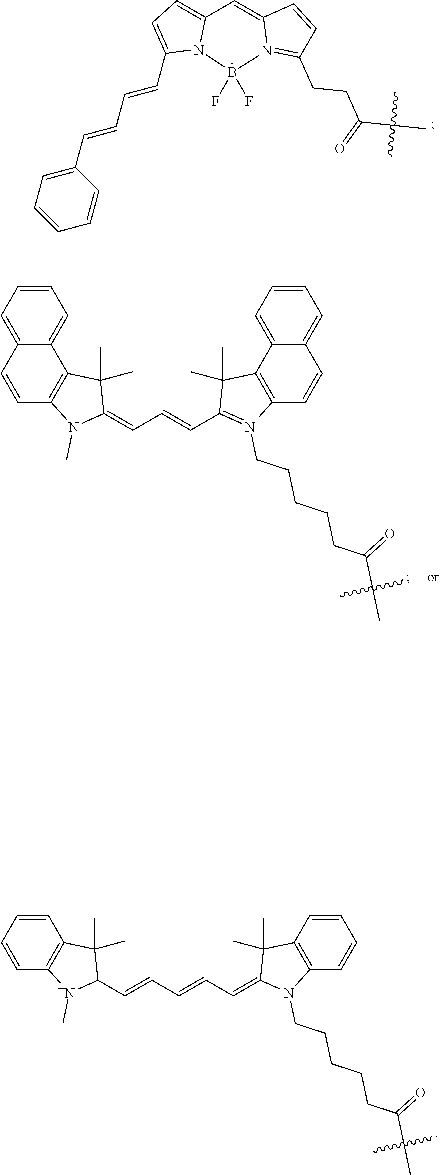

The efficiency of the FRET process depends, in part, on characteristics of the chromophores. Specifically, high efficiency FRET requires a large overlap between the absorbance spectrum of the donor chromophore and the emission spectrum of the acceptor chromophore. Additionally, the distance and orientation of the chromophores plays an important role. FRET efficiency is inversely proportional to the 6th power of the distance between the chromophores and the angle of the transition dipole moment should substantially align to be parallel (i.e., be near to 0° or 180°). Accordingly, in certain embodiments, covalent attachments of a first and a second chromophore to the polymer backbone are selected so distance between the first and second chromophore is minimized and transition dipole moments substantially align. The efficiency of FRET can be expressed according to the following equation:

$${E}_{FRET =} \frac{{Ro}^{6}}{{Ro}^{6} + {R}^{6}}$$

wherein E_{FRET} is FRET efficiency, R is the distance between chromophores, and Ro is expressed according to the following equation:

Ro=(8.8×10²³JK²Qₒn⁻⁴)^{1/6}

wherein J is the spectral overlap of the absorbance spectrum of the acceptor and the emission spectrum of the donor, Qₒ is donor quantum efficiency, n⁻⁴, is the index of medium between the donor and acceptor (constant), and K² is the dipole directions matching.

Accordingly, one embodiment provides a polymer compound comprising an acceptor chromophore having an acceptor transition dipole moment and being covalently linked to a polymer backbone, and a donor chromophore having a donor transition dipole moment and being covalently linked to the polymer backbone, wherein the polymer compound adopts a confirmation in solution at physiological conditions wherein the effective distance between the acceptor chromophore and the donor chromophore is less than about 50.0 nm and the acceptor transition dipole and the donor transition dipole are substantially parallel.

In some embodiments, the effective distance between the acceptor chromophore and the donor chromophore is less than about 25.0 nm. In some embodiments, the effective distance between the acceptor chromophore and the donor chromophore is less than about 10.0 nm. In some embodiments, the effective distance between the acceptor chromophore and the donor chromophore is less than about 30.0 nm, less than about 27.0 nm, less than about 22.0 nm, less than about 20.0 nm, less than about 17.0 nm, less than about 15.0 nm, less than about 12.0 nm, less than about 11.0 nm, less than about 9.0 nm, less than about 8.0 nm, less than about 7.0 nm, less than about 6.0 nm, less than about 5.0 nm, less than about 4.0 nm, less than about 3.0 nm, less than about 2.0 nm, or less than about 1.0 nm.

In some embodiments, the acceptor chromophore is a fluorescent dye moiety. In certain embodiments, the donor chromophore is a fluorescent dye moiety. In certain related embodiments, the acceptor chromophore and the donor chromophore are both fluorescent dye moieties.

In some embodiments, the angle between the acceptor transition dipole moment and the donor transition dipole moment ranges from 120° to 180°. For example, in some embodiments, the angle between the acceptor transition dipole moment and the donor transition dipole moment ranges from 1.25° to 180°, from 130° to 180°, from 140° to 180°, from 150° to 180°, from 160° to 180°, from 170° to 180°, from 172° to 180°, from 175° to 180°, or from 177′ to 180°.

In certain embodiments, the angle between the acceptor transition dipole moment and the donor transition dipole moment ranges from 0° to 60°. For example, For example, in some embodiments, the angle between the acceptor transition dipole moment and the donor transition dipole moment ranges from 0° to 50°, from 0° to 40°, from 0° to 30°, from 0° to 20°, from 0° to 10°, from 0° to 8°, from 0° to 5°, from 0° to 3°, or from 0° to 2°.

In embodiments, M¹ is a donor chromophores and M² is an acceptor chromophores. In such embodiments, M¹ and M² are selected based on the desired optical properties, for example based on a desired Stoke's shift, absorbance/emission overlap, a particular color and/or fluorescence emission wavelength. In some embodiments, M¹ (or alternatively M²) is the same at each occurrence; however, it is important to note that each occurrence of M¹ or M² need not be an identical M¹ or M², respectively. Certain embodiments include compounds wherein M¹ is not the same at each occurrence. Some embodiments include compounds wherein M² is not the same at each occurrence.

In some embodiments, M¹ and M² are selected to have absorbance and/or emission characteristics for use in FRET methods. For example, in such embodiments the different Wand M² moieties are selected such that M¹ has an absorbance of radiation at one wavelength that induces an emission of radiation by M² at a different wavelength by a FRET mechanism. Exemplary M¹ and M² moieties can be appropriately selected by one of ordinary skill in the art based on the desired end use.

Each respective M¹ and M² may be attached to the remainder of the molecule from any position (i.e., atom) on M¹ or M². One of skill in the art will recognize means for attaching M¹ or M² to the remainder of molecule. Exemplary methods include the “click” reactions described herein.

In some embodiments, M¹ or M² are FRET, fluorescent or colored moieties. Any fluorescent and/or colored moiety may be used to form a FRET donor-acceptor pair, for examples those known in the art and typically employed in colorimetric, UV, and/or fluorescent assays may be used. In some embodiments, M¹ or M², or both are, at each occurrence, independently fluorescent or colored. Examples of M¹ or M² moieties which are useful in various embodiments of the invention include, for example: Xanthene derivatives (e.g., fluorescein, rhodamine, Oregon green, eosin or Texas red); Cyanine derivatives (e.g., cyanine, indocarbocyanine, oxacarbocyanine, thiacarbocyanine or merocyanine); Squaraine derivatives and ring-substituted squaraines, including Seta, SeTau, and Square dyes; Naphthalene derivatives (e.g., dansyl and prodan derivatives); Coumarin derivatives; oxadiazole derivatives (e.g., pyridyloxazole, nitrobenzoxadiazole, or benzoxadiazole); Anthracene derivatives (e.g., anthraquinones, including DRAQ5, DRAQ7, and CyTRAK Orange); Pyrene derivatives such as cascade blue; Oxazine derivatives (e.g., Nile red, Nile blue, cresyl violet, oxazine 170); Acridine derivatives (e.g., proflavin, acridine orange, acridine yellow); Arylmethine derivatives: auramine, crystal violet, malachite green; and Tetrapyrrole derivatives (e.g., porphin, phthalocyanine, or bilirubin). Other exemplary M moieties include: Cyanine dyes, xanthate dyes (e.g., Hex, Vic, Nedd, Joe, or Tet); Yakima yellow; Redmond red; tamra; texas red; and alexa Fluor® dyes.

In still other embodiments of any of the foregoing, M¹ or M², or both, at each occurrence, independently comprise three or more aryl or heteroaryl rings, or combinations thereof, for example four or more aryl or heteroaryl rings, or combinations thereof, or even five or more aryl or heteroaryl rings, or combinations thereof. In some embodiments, M¹ or M², or both, at each occurrence, independently comprise six aryl or heteroaryl rings, or combinations thereof. In further embodiments, the rings are fused. For example in some embodiments, M¹ or M² or both, at each occurrence, independently comprise three or more fused rings, four or more fused rings, five or more fused rings, or even six or more fused rings.

In some embodiments, M¹ or M² or both, are cyclic. For example, in some embodiments M¹ or M² or both, are carbocyclic. In other embodiment, M¹ or M² or both are heterocyclic. In still other embodiments of the foregoing, M¹ or M² or both, at each occurrence, independently comprise an aryl moiety. In some of these embodiments, the aryl moiety is multicyclic. In other more specific examples, the aryl moiety is a fused-multicyclic aryl moiety, for example which may comprise at least 3, at least 4, or even more than 4 aryl rings.

In other embodiments of any of the foregoing compounds of structure (I), (II), (IA), (IB), (IC), (ID), or (IE) M¹ or M² or both, at each occurrence, independently comprise at least one heteroatom. For example, in some embodiments, the heteroatom is nitrogen, oxygen or sulfur.

In still more embodiments of any of the foregoing, M¹ or M² or both, at each occurrence, independently comprise at least one substituent. For example, in some embodiments the substituent is a fluoro, chloro, bromo, iodo, amino, alkylamino, arylamino, hydroxy, sulfhydryl, alkoxy, aryloxy, phenyl, aryl, methyl, ethyl, propyl, butyl, isopropyl, t-butyl, carboxy, sulfonate, amide, or formyl group.

In some even more specific embodiments of the foregoing, M¹ or M² or both are, at each occurrence, independently a dimethylaminostilbene, quinacridone, fluorophenyl-dimethyl-BODIPY, hi s-fluorophenyl-BODIPY, acridine, terrylene, sexiphenyl, porphyrin, benzopyrene, (fluorophenyl-dimethyl-difluorobora-diaza-indacene)phenyl, (bis-fluorophenyl-difluorobora-diaza-indacene)phenyl, quaterphenyl, bi-benzothiazole, ter-benzothiazole, bi-naphthyl, bi-anthracyl, squaraine, squarylium, 9, 10-ethynylanthracene, or ter-naphthyl moiety. In other embodiments, M¹ or M² or both are, at each occurrence, independently p-terphenyl, perylene, azobenzene, phenazine, phenanthroline, acridine, thioxanthrene, chrysene, rubrene, coronene, cyanine, perylene imide, or perylene amide, or a derivative thereof. In still more embodiments, M¹ or M² or both are, at each occurrence, independently a coumarin dye, resorufin dye, dipyrrometheneboron difluoride dye, ruthenium bipyridyl dye, energy transfer dye, thiazole orange dye, polymethine, or N-aryl-1,8-naphthalimide dye. In certain embodiments, M¹ and M² are, at each occurrence, independently boron-dipyrromethene, rhodamine, cyanine, pyrene, perylene, perylene monoimide, or 6-FAM or a derivative thereof.

In still more embodiments of any of the foregoing, M¹ at each occurrence is the same. In other embodiments, each M¹ is different. In still more embodiments, one or more M¹ is the same and one or more M¹ is different.

In still more embodiments of any of the foregoing, M² at each occurrence is the same. In other embodiments, each M² is different. In still more embodiments, one or more M² is the same and one or more M² is different.

In some embodiments, M¹ or M² or both are, at each occurrence, independently boron-dipyrromethene, rhodamine, cyanine, pyrene, perylene, perylene monoimide, or 6-FAM or a derivative thereof. In some other embodiments, M¹ and M² at each occurrence, independently have one of the following structures:

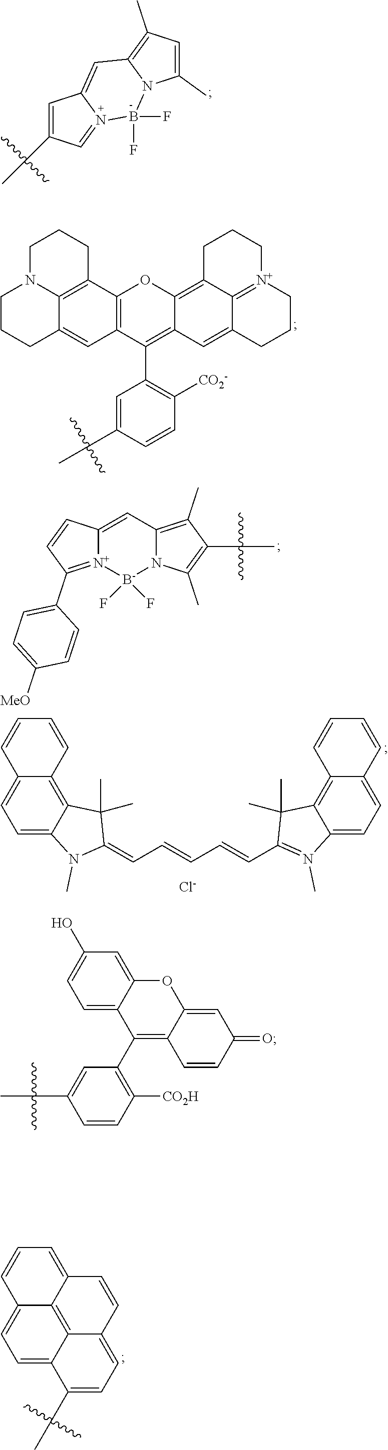

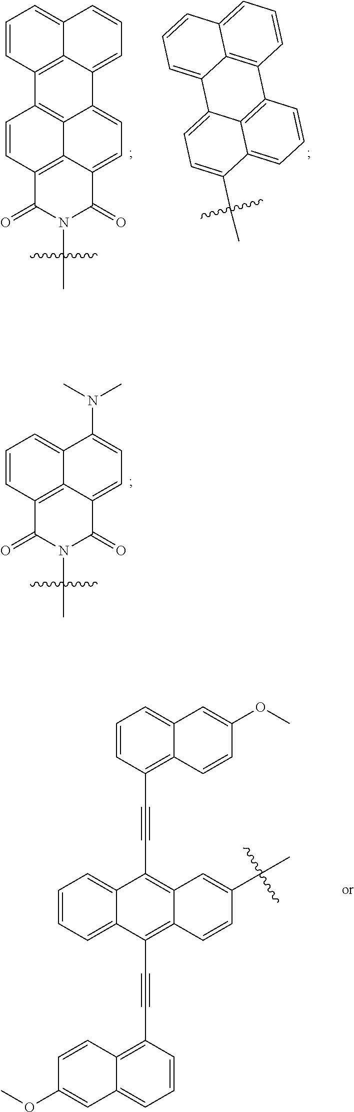

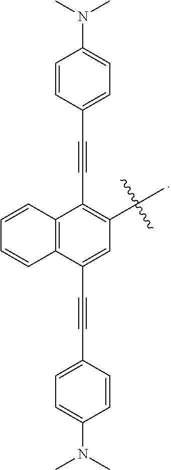 [SMILES: *c1cc(C#Cc2ccc(N(C)C)cc2)c2ccccc2c1C#Cc1ccc(N(C)C)cc1]

As is understood, the compounds described herein can be formed using any suitable methods, such as those described in US 2017/0292957, US 2016/0208100, US 2016/0341736, US 2018/0065998, US 2018/0079909, and US 2019/0016898 which are incorporated by referenced in their entirety for such teachings.

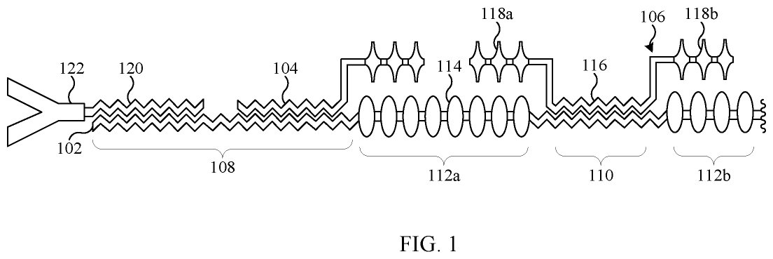

A schematic version of an illustrative capture probe 102 and illustrative detectable probes 104, 106 are shown in FIG. 1. The capture probe 102 comprises (1) a first segment comprising a first polynucleotide 108 having a first sequence, and (2) a second segment conjugated to the first segment, the second segment comprising a plurality of alternating second polynucleotides 110 and first polymers 112a, 112b, each of the first polymers comprising a first chromophore 114, each of the second polynucleotides comprising a second sequence. In some embodiments, the capture probe is a compound of structure (III), as described above.

Also present are a plurality of detectable probes 104, 106, each of the plurality of detectable probes comprising a third polynucleotide 116 covalently bound to at least one a second polymer comprising a second chromophore 118a, 118b. In embodiments, the third polynucleotide has a third sequence that has at least 90% complementarity to the second sequence.

Accordingly, described herein are compositions, comprising: (1) a capture probe comprising: (i) a first segment comprising a first polynucleotide having a first sequence; and (ii) a second segment conjugated to the first segment, the second segment comprising a plurality of alternating second polynucleotides and first polymers, each of the first polymers comprising a first chromophore, each of the second polynucleotides comprising a second sequence; and (2) a plurality of detectable probes, each of the plurality of detectable probes comprising a third polynucleotide covalently bound to at least one a second polymer comprising a second chromophore, the third polynucleotide having a third sequence that has at least 90% complementarity to the second sequence.

In embodiments, the second sequence has at least 92% complementarity to the third sequence. In some embodiments, the second sequence has at least 95% complementarity to the third sequence. In some embodiments, the second sequence has at least 96% complementarity to the third sequence. In some embodiments, the second sequence has at least 97% complementarily to the third sequence. In some embodiments, the second sequence has at least 98% complementarity to the third sequence. In some embodiments, the second sequence has at least 99% complementarity to the third sequence. In some embodiments, a triplex structure is formed by the polynucleotides of two detectable probes and the polynucleotide of the capture probe.

As shown in FIG. 1, in some embodiments, each of the first polymers comprise a plurality of the first chromophores. Similarly, in some embodiments, each of the detectable probes comprise a plurality of the second chromophores. In some embodiments, a second polymer 118a, 118b, is covalently bound to each end of the third polynucleotide 116 of at least one of the detectable probes 106.

In some embodiments, such as the embodiment shown in FIG. 1, the first chromophore is an acceptor chromophore. In other embodiments, the first chromophore is a donor chromophore. As shown in FIG. 1, in some embodiments, the second chromophore is a donor chromophore. In other embodiments, the second chromophore is a donor chromophore.

In embodiments, the third polynucleotide has a length ranging from 10 nucleotides to 40 nucleotides. In some embodiments, the third polynucleotide has a length ranging from 10 nucleotides to 30 nucleotides. In some embodiments, the third polynucleotide has a length ranging from 15 nucleotides to 25 nucleotides.

In embodiments, the third polynucleotide comprises a first portion, a second portion adjacent to the first portion, and a third portion separated from the first portion by the second portion. In some such embodiments, the third polynucleotide has a higher degree of complementarily to a portion of a second sequence than the degree of complementarity of the first portion to the third portion. Thus, the third polynucleotide will preferentially hybridize to the second sequence rather than forming a hairpin structure. In embodiments, at least a portion of the detectable probes have the structure (I), (II), (IIa), (IIb), or a combination thereof, described above. In some embodiments, at least one of the detectable probes has the structure (I) as described above. In some embodiments, at least one of the detectable probes has the structure (II) as described above. In some embodiments, at least one of the detectable probes has the structure (IIa) as described above. In some embodiments, at least one of the detectable probes has the structure (IIb) as described above.

In embodiments, each of the detectable probes have the structure (I), (II), (IIa), (IIb), or a combination thereof, described above. In some embodiments, each of the detectable probes have the structure (I) as described above. In some embodiments, each of the detectable probes have the structure (II) as described above. In some embodiments, each of the detectable probes have the structure (IIa) as described above. In some embodiments, each of the detectable probes have the structure (IIb) as described above.

As also illustrated in FIG. 1, a fourth polynucleotide 120 covalently bound to a targeting moiety 122. In other embodiments, a targeting moiety is covalently bound to the capture probe.

In embodiments, a composition comprises a capture probe and a detectable probe as described herein. Accordingly, described herein are compositions comprising: (1) a capture probe comprising: (i) a first segment comprising a first polynucleotide having a first sequence; and (ii) a second segment conjugated to the first segment, the second segment comprising a plurality of second polynucleotides each comprising a second sequence; and (2) a plurality of detectable probes, each of the plurality of detectable probes comprising a third polynucleotide having a first end and a second end, at least one of the first end and the second end being covalently bound to a first polymer comprising a chromophore, the third polynucleotide having a third sequence that has at least 90% complementarity to the second sequence.

In embodiments, the third polynucleotide has a length ranging from 10 nucleotides to 40 nucleotides. In some embodiments, the third polynucleotide has a length ranging from 10 nucleotides to 30 nucleotides. In some embodiments, the third polynucleotide has a length ranging from 15 nucleotides to 25 nucleotides.

In embodiments, the second sequence has at least 92% complementarity to the third sequence. In some embodiments, the second sequence has at least 95% complementarity to the third sequence. In some embodiments, the second sequence has at least 96% complementarity to the third sequence. In some embodiments, the second sequence has at least 97% complementarity to the third sequence. In some embodiments, the second sequence has at least 98% complementarily to the third sequence. In some embodiments, the second sequence has at least 99% complementarity to the third sequence.

In some embodiments, each of the first polymers comprise a plurality of chromophores. Similarly, in some embodiments, each of the second polymers comprises a plurality of chromophores. In some embodiments, a second type of detectable probe is also present. In some such embodiments, the detectable probe comprises a third polynucleotide bound to a first polymer or a second polymer.

In embodiments, at least a portion of the detectable probes have the structure (I), (II), (IIa), (IIb), or a combination thereof, described above. In some embodiments, at least one of the detectable probes has the structure (I) as described above. In some embodiments, at least one of the detectable probes has the structure (II) as described above. In some embodiments, at least one of the detectable probes has the structure (IIa) as described above. In some embodiments, at least one of the detectable probes has the structure (Fib) as described above.

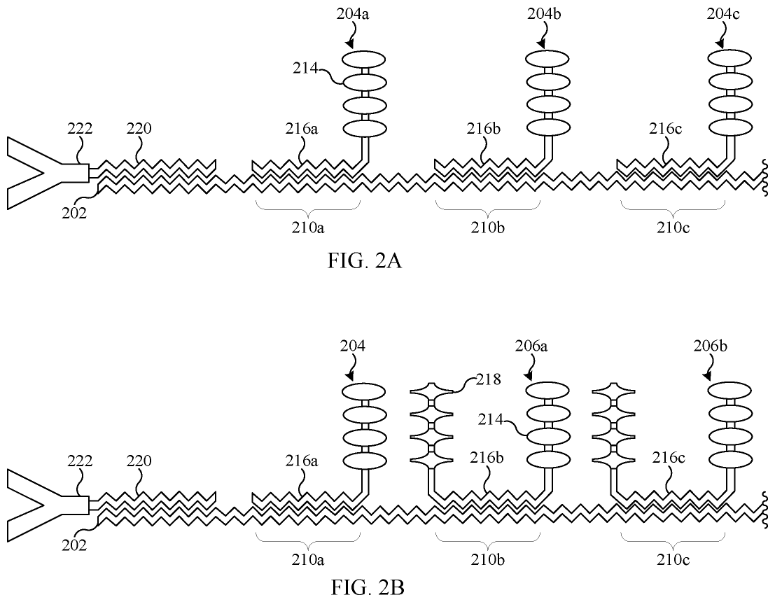

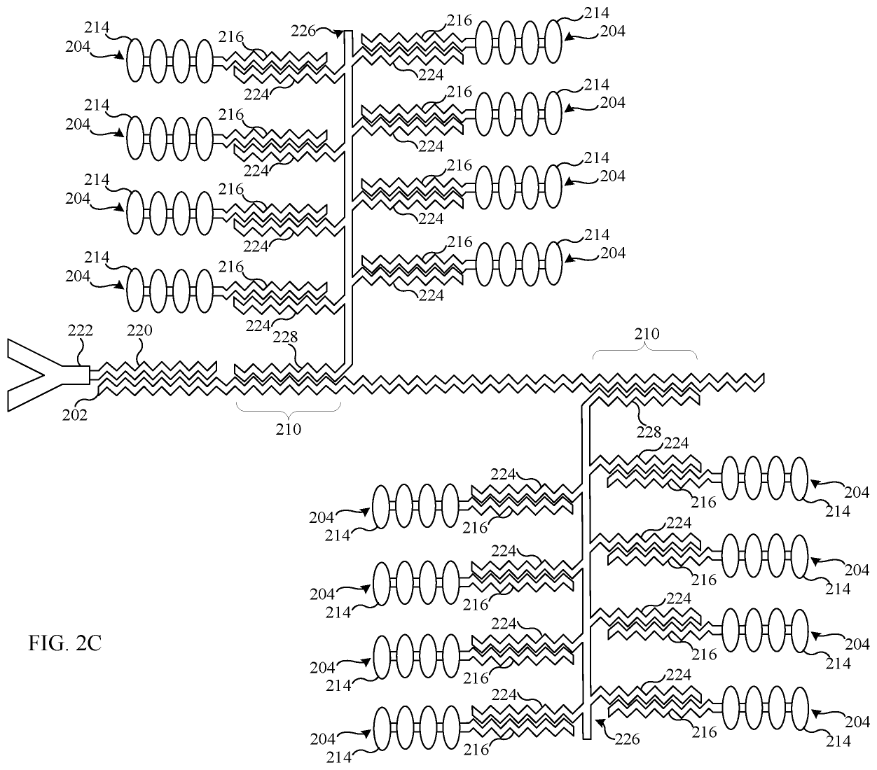

In embodiments, each of the detectable probes have the structure (I), (II), (IIa), (IIb), or a combination thereof described above. In some embodiments, each of the detectable probes have the structure (I) as described above (as illustrated in FIG. 2A). In some embodiments, each of the detectable probes have the structure (II) as described above. In some embodiments, each of the detectable probes have the structure (IIa) as described above. In some embodiments, each of the detectable probes have the structure (IIb) as described above.

In embodiments, the fourth polynucleotide is covalently bound to a targeting moiety. In various embodiments, the fourth polynucleotide having a fourth sequence that has at least 90% complementarity to the first sequence. In some embodiments, the fourth sequence has at least 92% complementarity to the first sequence. In some embodiments, the fourth sequence has at least 95% complementarity to the first sequence. In some embodiments, the fourth sequence has at least 96% complementarity to the first sequence. In some embodiments, the fourth sequence has at least 97% complementarity to the first sequence. In some embodiments, the fourth sequence has at least 98% complementarity to the first sequence. In some embodiments, the fourth sequence has at least 99% complementarity to the first sequence. In other embodiments, a targeting moiety is covalently bound to the capture probe.

A schematic version of another embodiment of an illustrative capture probe 202 and illustrative detectable probes 204a, 204b, 204c are shown in FIG. 2A. The capture probe 102 comprises (1) a first segment comprising a first polynucleotide having a first sequence, and (2) a second segment conjugated to the first segment, the second segment comprising a plurality of second polynucleotides 210a, 210b, 210c each comprising a second sequence.

Also present are a plurality of detectable probes 204a, 204b, 204c, each of the plurality of detectable probes comprising a third polynucleotide 216a, 216b, 216c. The third polynucleotides are covalently bound on one end to a first polymer comprising a chromophore 214. In embodiments, the third polynucleotide having a third sequence that has at least 90% complementarity to the second sequence.

Accordingly, described herein are compositions comprising: (1) a capture probe comprising: (i) a first segment comprising a first polynucleotide having a first sequence; and (ii) a second segment conjugated to the first segment, the second segment comprising a plurality of second polynucleotides each comprising a second sequence; and (2) a plurality of detectable probes, each of the plurality of detectable probes comprising a third polynucleotide having a first end and a second end, the first end being covalently bound to a first polymer comprising a chromophore, the third polynucleotide having a third sequence that has at least 90% complementarity to the second sequence.

In embodiments, the third polynucleotide has a length ranging from 10 nucleotides to 40 nucleotides. In some embodiments, the third polynucleotide has a length ranging from 10 nucleotides to 30 nucleotides. In some embodiments, the third polynucleotide has a length ranging from 15 nucleotides to 25 nucleotides.

In embodiments, the second sequence has at least 92% complementarity to the third sequence. In some embodiments, the second sequence has at least 95% complementarity to the third sequence. In some embodiments, the second sequence has at least 96% complementarity to the third sequence. In some embodiments, the second sequence has at least 97% complementarity to the third sequence. In some embodiments, the second sequence has at least 98% complementarily to the third sequence. In some embodiments, the second sequence has at least 99% complementarity to the third sequence. In some embodiments, a triplex structure is formed by the polynucleotides of two detectable probes and the polynucleotide of the capture probe.

As shown in FIG. 2A, in some embodiments, each of the first polymers comprise a plurality of chromophores. In some embodiments, a second type of detectable probe is also present. In some such embodiments, the detectable probe comprises a third polynucleotide bound to a first polymer and/or a second polymer.

In embodiments, at least a portion of the detectable probes have the structure (I), (II), (IIa), (IIb), or a combination thereof, described above. In some embodiments, at least one of the detectable probes has the structure (I) as described above. In some embodiments, at least one of the detectable probes has the structure (II) as described above. In some embodiments, at least one of the detectable probes has the structure (IIa) as described above. In some embodiments, at least one of the detectable probes has the structure (Fib) as described above.

In embodiments, each of the detectable probes have the structure (I), (II), (IIa), (IIb), or a combination thereof, described above. In some embodiments, each of the detectable probes have the structure (I) as described above (as illustrated in FIG. 2A). In other embodiments, each of the detectable probes have the structure (II) as described above. In some embodiments, each of the detectable probes have the structure (IIa) as described above. In some embodiments, each of the detectable probes have the structure (IIb) as described above.

As also illustrated in FIG. 2A, a fourth polynucleotide 220 covalently bound to a targeting moiety 222. In various embodiments, the fourth polynucleotide having a fourth sequence that has at least 90% complementarity to the first sequence. In some embodiments, the fourth sequence has at least 92% complementarity to the first sequence. In some embodiments, the fourth sequence has at least 95% complementarity to the first sequence. In some embodiments, the fourth sequence has at least 96% complementarity to the first sequence. In some embodiments, the fourth sequence has at least 97% complementarity to the first sequence. In some embodiments, the fourth sequence has at least 98% complementarity to the first sequence. In some embodiments, the fourth sequence has at least 99% complementarity to the first sequence. In other embodiments, a targeting moiety is covalently bound to the capture probe.

A schematic version of another embodiment of an illustrative capture probe 202 and illustrative detectable probes 204, 206a, 206b are shown in FIG. 2B. The capture probe 102 comprises (1) a first segment comprising a first polynucleotide having a first sequence, and (2) a second segment conjugated to the first segment, the second segment comprising a plurality of second polynucleotides 210a, 210b, 210c each comprising a second sequence.

Also present are a plurality of detectable probes 204, 206a, 206b, each of the plurality of detectable probes comprising a third polynucleotide 216a, 216b, 216c. The third polynucleotides are covalently bound on one end to a first polymer comprising an acceptor chromophore 214, and on the other end to a second polymer comprising a donor chromophore 218. In other words, each of the plurality of detectable probes comprising a third polynucleotide having a first end and a second end, the first end being covalently bound to a first polymer comprising an acceptor chromophore, the second end being covalently bound to a second polymer comprising a donor chromophore. In embodiments, the third polynucleotide having a third sequence that has at least 90% complementarity to the second sequence.

Accordingly, described herein are compositions comprising: (1) a capture probe comprising: (i) a first segment comprising a first polynucleotide having a first sequence; and (ii) a second segment conjugated to the first segment, the second segment comprising a plurality of second polynucleotides each comprising a second sequence; and (2) a plurality of detectable probes, each of the plurality of detectable probes comprising a third polynucleotide having a first end and a second end, the first end being covalently bound to a first polymer comprising an acceptor chromophore, the second end being covalently bound to a second polymer comprising a donor chromophore, the third polynucleotide having a third sequence that has at least 90% complementarity to the second sequence.

In embodiments, the second sequence has at least 92% complementarity to the third sequence. In some embodiments, the second sequence has at least 95% complementarily to the third sequence. In some embodiments, the second sequence has at least 96% complementarity to the third sequence. In some embodiments, the second sequence has at least 97% complementarity to the third sequence. In some embodiments, the second sequence has at least 98% complementarily to the third sequence. In some embodiments, the second sequence has at least 99% complementarity to the third sequence. In some embodiments, a triplex structure is formed by the polynucleotides of two detectable probes and the polynucleotide of the capture probe.

As shown in FIG. 2B, in some embodiments, each of the first polymers comprise a plurality of chromophores. Similarly, in some embodiments, each of the second polymers comprises a plurality of chromophores. In some embodiments, a second type of detectable probe 204 is also present. In some such embodiments, the detectable probe 204 comprises a third polynucleotide bound to a first polymer or a second polymer.

In embodiments, the third polynucleotide has a length ranging from 10 nucleotides to 40 nucleotides. In some embodiments, the third polynucleotide has a length ranging from 10 nucleotides to 30 nucleotides. In some embodiments, the third polynucleotide has a length ranging from 15 nucleotides to 25 nucleotides.

In embodiments, at least a portion of the detectable probes have the structure (I), (II), (IIa), (IIb), or a combination thereof, described above. In some embodiments, at least one of the detectable probes has the structure (I) as described above. In some embodiments, at least one of the detectable probes has the structure (II) as described above. In some embodiments, at least one of the detectable probes has the structure (IIa) as described above. In some embodiments, at least one of the detectable probes has the structure (IIb) as described above.

In embodiments, each of the detectable probes have the structure (I), (II), (IIa), (IIb), or a combination thereof, described above. In some embodiments, each of the detectable probes have the structure (I) as described above. In some embodiments, each of the detectable probes have the structure (II) as described above. In some embodiments, each of the detectable probes have the structure (IIa) as described above. In some embodiments, each of the detectable probes have the structure (IIb) as described above.

As also illustrated in FIG. 2B, a fourth polynucleotide 220 covalently bound to a targeting moiety 222. In various embodiments, the fourth polynucleotide having a fourth sequence that has at least 90% complementarity to the first sequence. In some embodiments, the fourth sequence has at least 92% complementarity to the first sequence. In some embodiments, the fourth sequence has at least 95% complementarity to the first sequence. In some embodiments, the fourth sequence has at least 96% complementarity to the first sequence. In some embodiments, the fourth sequence has at least 97% complementarity to the first sequence. In some embodiments, the fourth sequence has at least 98% complementarity to the first sequence. In some embodiments, the fourth sequence has at least 99% complementarity to the first sequence. In other embodiments, a targeting moiety is covalently bound to the capture probe.

Another embodiment is illustrated in FIG. 2C. The capture probe 102 comprises (1) a first segment comprising a first polynucleotide having a first sequence, and (2) a second segment conjugated to the first segment, the second segment comprising a plurality of second polynucleotides 210 each comprising a second sequence.

Also present are a plurality of branched linkers 226, and a plurality of detectable probes 204. Each of the plurality of detectable probes comprise a third polynucleotide 216 (having a third sequence) and each of the plurality of branched linkers comprise a fourth polynucleotide 228 (having a fourth sequence) and a fifth polynucleotide 224 (having a fifth sequence). The third polynucleotides are covalently bound on one end to a first polymer comprising a chromophore 214.

In embodiments, the second sequence has at least 90% complementarity to the fourth sequence. In embodiments, the second sequence has at least 92% complementarity to the fourth sequence. In some embodiments, the second sequence has at least 95% complementarity to the fourth sequence. In some embodiments, the second sequence has at least 96% complementarity to the fourth sequence. In some embodiments, the second sequence has at least 97% complementarity to the fourth sequence. In some embodiments, the second sequence has at least 98% complementarity to the fourth sequence. In some embodiments, the second sequence has at least 99% complementarity to the fourth sequence.

In embodiments, the third sequence has at least 90% complementarity to the fifth sequence. In embodiments, the third sequence has at least 92% complementarity to the fifth sequence. In some embodiments, the third sequence has at least 95% complementarily to the fifth sequence. In some embodiments, the third sequence has at least 96% complementarity to the fifth sequence. In some embodiments, the third sequence has at least 97% complementarity to the fifth sequence. In some embodiments, the third sequence has at least 98% complementarity to the fifth sequence. In some embodiments, the third sequence has at least 99% complementarity to the fifth sequence.

In embodiments, the second, third, fourth, and/or fifth polynucleotide has a length ranging from 10 nucleotides to 40 nucleotides. In some embodiments, the second, third, fourth, and/or fifth polynucleotide has a length ranging from 10 nucleotides to 30 nucleotides. In some embodiments, the second, third, fourth, and/or fifth polynucleotide has a length ranging from 15 nucleotides to 25 nucleotides. In some embodiments, a triplex structure is formed by the polynucleotides of two detectable probes and the polynucleotide of the capture probe.

In some embodiments, each of the first polymers comprise a plurality of chromophores.

In embodiments; at least a portion of the detectable probes have the structure (I). In some embodiments, at least one of the detectable probes has the structure (1) as described above. In some embodiments, each of the detectable probes have the structure (I) as described above.

In some embodiments, a sixth polynucleotide 220 is covalently bound to a targeting moiety 222. In various embodiments, the sixth polynucleotide having a sixth sequence that has at least 90% complementarity to the first sequence. In some embodiments, the sixth sequence has at least 92% complementarity to the first sequence. In some embodiments; the sixth sequence has at least 95% complementarity to the first sequence. In some embodiments, the sixth sequence has at least 96% complementarity to the first sequence. In some embodiments, the sixth sequence has at least 97% complementarity to the first sequence. In some embodiments, the sixth sequence has at least 98% complementarily to the first sequence. In some embodiments, the sixth sequence has at least 99% complementarity to the first sequence. In other embodiments, a targeting moiety is covalently bound to the capture probe.

Accordingly, described herein are compositions comprising: (1) a capture probe comprising: (i) a first segment comprising a first polynucleotide having a first sequence; and (ii) a second segment conjugated to the first segment, the second segment comprising a plurality of second polynucleotides each comprising a second sequence; and (2) a plurality of detectable probes, each of the plurality of detectable probes comprising a third polynucleotide having a first end and a second end, the first end being covalently bound to a first polymer comprising a chromophore, the third polynucleotide having a third sequence; (3) a plurality of branched linkers comprising a polymer backbone bound to a fourth polynucleotide and a plurality of fifth polynucleotides, the fourth polynucleotide having a fourth sequence that has at least 90% complementarity to the second sequence, each of the fifth polynucleotides having a fifth sequence that has at least 90% complementarity to the third sequence.

Also described herein are kits comprising the capture probes, detectable probes, or both described herein. In some such embodiments, a kit of the present disclosure further comprises instructions for use of the compound, composition, or detectable probe for identification of a target nucleotide sequence.

Further described are methods for identifying the presence of a target analyte, comprising: producing a mixture by contacting a sample with the composition described herein under assay conditions; and imaging the mixture under detection conditions. In some embodiments, imaging the mixture comprises: exciting the donor fluorophores at a first wavelength; and detecting emission of the acceptor fluorophores at a second wavelength.

### EXAMPLES

### General Methods

Mass spectral analysis was performed on a Waters/Micromass Quattro micro MS/MS system (in MS only mode) using MassLynx 4.1 acquisition software. Mobile phase used for LC/MS on dyes was 100 mM 1,1,1,3,3,3-hexafluoro-2-propanol (HFIP), 8.6 mM triethylamine (TEA), pH 8. Phosphoramidites and precursor molecules were also analyzed using a Waters Acquity UHPLC system with a 2.1 mm×50 mm Acquity BEH-C18 column held at 45° C., employing an acetonitrile/water mobile phase gradient. Molecular weights for monomer intermediates were obtained using tropylium cation infusion enhanced ionization on a Waters/Micromass Quattro micro MS/MS system (in MS only mode). Excitation and emission profiles experiments were recorded on a Cary Eclipse spectra photometer.

All reactions were carried out in oven dried glassware under a nitrogen atmosphere unless otherwise stated. Commercially available DNA synthesis reagents were purchased from Glen Research (Sterling, VA). Anhydrous pyridine, toluene, dichloromethane, diisopropylethyl amine, triethylamine, acetic acid, pyridine, and THE were purchased from Aldrich. All other chemicals were purchased from Aldrich or TCI and were used as is with no additional purification.

### Example 1

### Synthesis of Dyes with Ethylene Glycol Spacer

Compounds with ethylene oxide linkers were prepared as followed:

The oligofluoroside constructs (e.g., compounds of structure (I), (II), or (III)) were synthesized on an Applied Biosystems 394 DNA/RNA synthesizer on 1 μmol scale and possessed a 3′-phosphate group or 3′-S₂—(CH₂)₆—OH group or any of the other groups described herein. Synthesis was performed directly on CPG beads or on Polystyrene solid support using standard phopshoporamadite chemistry. The oligofluorosides were synthesized in the 3′ to 5′ direction using standard solid phase DNA methods, and coupling employed standard β-cyanoethyl phosphoramidite chemistry. Fluoroside and nucleoside phosphoramidites and spacers (e.g., hexaethyloxy-glycol phosphoramidite, triethyloxy-glycol phosphoramidite, polyethylene glycol phosphoramidite) and linkers (e.g., 5′-amino-Modifier Phosphoramidite and thiol—Modifiers S2 Phosphoramidite) were dissolved in acetonitrile to make 0.1 M solutions, and were added in successive order using the following synthesis cycle: 1) removal of the 5′-dimethoxytrityl protecting group with dichloroacetic acid in dichloromethane, 2) coupling of the next phosphoramidite with activator reagent in acetonitrile, 3) oxidation of P(III) to form stable P(v) with iodine/pyridine/water, and 4) capping of any unreacted 5′-hydroxyl groups with acetic anhydride/1-methylimidizole/acetonitrile. The synthesis cycle was repeated until the full length oligofluoroside construct was assembled. At the end of the chain assembly, the monomethoxytrityl (MMT) group or dimethoxytrityl (DMT) group was removed with dichloroacetic acid in dichloromethane.

The compounds were provided on controlled-pore glass (CPG) support at 0.2 umol scale in a labeled Eppendorf tube. 400 μL of 20-30% NH₄OH was added and mixed gently. Open tubes were placed at 55° C. for ˜5 minutes or until excess gases had been liberated, and then were closed tightly and incubated for 2 hrs (+/−15 min.). Tubes were removed from the heat block and allowed to reach room temperature, followed by centrifugation at 13,400 RPM for 30 seconds to consolidate the supernatant and solids. Supernatant was carefully removed and placed into a labeled tube, and then 150 μL acetonitrile was added to wash the support. After the wash was added to the tubes they were placed into a CentriVap apparatus at 40° C. until dried.

The products are characterized by ESI-MS, UV-absorbance, and fluorescence spectroscopy.

### Example 2

### Spectral Testing of Compounds

Dried compounds were reconstituted in 150 μL of 0.1M Na₂CO₃ buffer to make a ˜1 mM stock. The concentrated stock was diluted 50× in 0.1×PBS and analyzed on a NanoDrop UV spectrometer to get an absorbance reading. Absorbance readings were used along with the extinction coefficient (75,000 M⁻¹ cm⁻¹ for each FAM unit) and Beer's Law to determine an actual concentration of the stock.

From the calculated stock concentrations, ˜4 mL of a 5 μM solution was made in 0.1M Na₂CO₃ (pH 9) and analyzed in a 1×1 cm quartz cuvette on a Cary 60 UV spectrometer, using a spectral range of 300 nm to 700 nm, to gauge overall absorbance relative to the group. From these 5 uM solutions, a second dilution was made at either 50 nM or 25 nM (also in 0.1M Na₂CO₃, pH 9) for spectral analysis on a Cary Eclipse Fluorimeter. Excitation was set at 494 nm and emission spectra were collected from 499 to 700 nm.

### Example 3

### Signal Amplification Through Concatemerization

A biotinylated target probe (5′-B-heg-ATGCACAGTCG-dT-3′) (SEQ ID NO: 1) bound to a neutravidin bead alone and mixed with (1) a first probe (5′-CGA CGC TTA CAG-heg-F-(heghegheg-F)3-heg-CCG ACT GTG CA-dT-3′) (SEQ ID NO: 2) and a plurality of second probes (5′-CTGTAAGCGTCG-heg-F(heghegheg-F)9-heg-GACATTCGCAGC-dT-3′) (SEQ ID NO: 3), (2) a first probe (5′-CGA CGC TTA CAG-heg-F-(heghegheg-F)3-heg-CCG ACT GTG CA-dT-3′) (SEQ ID NO: 2), were analyzed using flow cytometry.

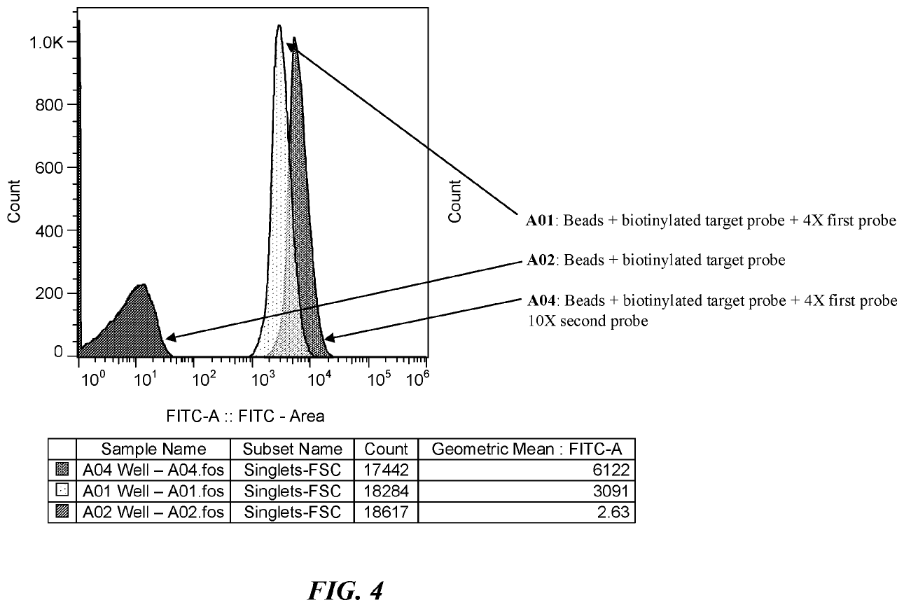

Stocks were heated using a thermal cycler to 94° C. for 10 min, then cooled and maintained at 37° C. until use. Five nanogram (ng) capture probe per sample was bound to 30 ul of neutravidin coated beads by incubation for 30 min at room temp in dark. Beads were washed with hybridization buffer (10 mM Tris-HCL, 4 mM MgCl, 15 mM KCl, pH 8.2) and centrifuged for 5 min at 400×g. Supernatant was removed and samples were resuspended in cell staining buffer. For conditions 1 and 2, 1 ul of 100 UM first probe was added and samples were incubated for 10 min at room temperature, followed by washing with hybridization buffer. Samples were centrifuged for 5 min at 400×g, supernatant was removed and samples were resupended in cell staining buffer or treated with second probe. For condition 2, 1 ul of 100 uM second probe was added and sample incubated for 10 min at room temperature, followed by washing with hybridization buffer. Sample centrifuged for 5 min at 400×g and supernatant was removed. Sample was resuspended in cell staining buffer and analyzed on the SA3800 flow cytometer. The results are shown in FIG. 4.

### Example 4

### Branched Amplification

A biotinylated target probe (5′-Biotin-heg-TTT CTT TGA GGT ITA GGA TTC-dT-3′) (SEQ ID NO: 4) 5 ng bound to a neutravidin bead is mixed with a target amplifier (3′-dT-AAA GAA ACT CCA AAT CCT AAG-GGT TGG CCT TAG GGT TC AGA-GGT TGG CCT TAG GGT TC AGA-heg-F-5′) (SEQ ID NO: 5) 200 pmol and a plurality of detection probes selected from: (1) 5′-CCA ACC GGA ATC CCA AG X-F-dT-3; (SEQ ID NO: 6), 5′-CCA. ACC GGA ATC CCA AG-heg-F(heghegheg-F)3-heg-dT-3′(SEQ ID NO: 7); (2) 5′-CCA ACC GGA ATC CCA AG X-F-dT-3′ (SEQ ID NO: 6), 5′-CCA ACC CGA ATC CCA AG-heg-F(heghegheg-F)4-heg-dT-3′(SEQ ID NO: 8); and (3) 5′-CCA ACC GGA ATC CCA AG-heg-F(heghegheg-F)9-heg-dT-3′(SEQ ID NO: 9).

Probe stocks were heated using a thermal cycler to 94° C. for 10 min, then cooled and maintained at 37° C. until use. Five ng capture probe per sample was bound to neutravidin beads by incubation for 30 min at room temp in the dark. Beads were washed with hybridization buffer, centrifuged for 5 min at 400×g and supernatant was removed. 200 pmol target amplifier was added and samples were incubated for 10 min at 37° C., followed by washing with hybridization buffer as described above. 400 pmole detection probe was added and samples were incubated for 10 min at 37° C. Samples were washed with hybridization buffer as described above and resuspended in cell staining buffer for analysis on the SA3800 flow cytometer.

### Example 5

### Branched Amplification

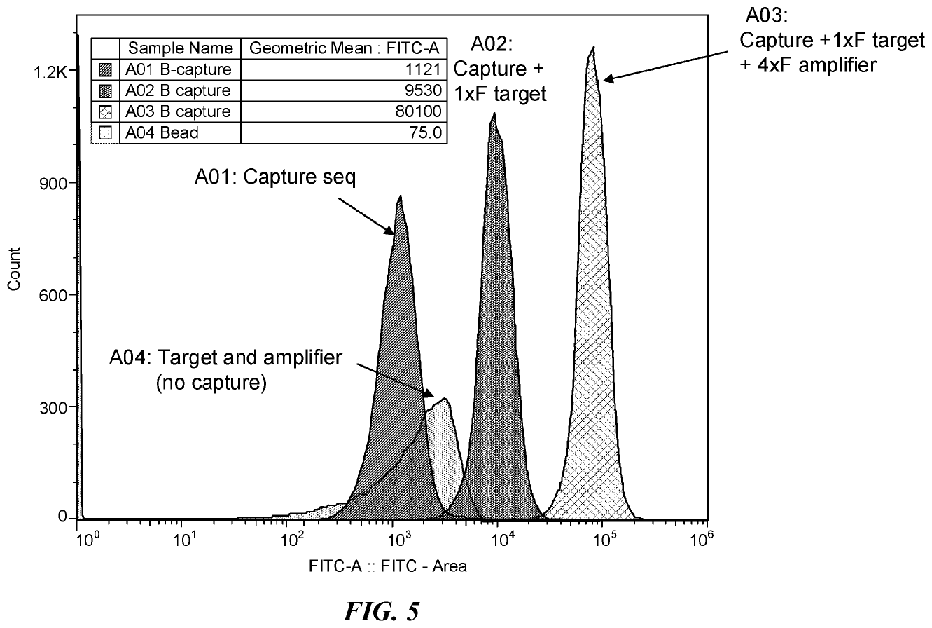

A capture probe mixed with (1) a target amplifier (3′-dT-AAA GAA ACT CCA AAT CCT AAG-GGT TGG CCT TAG GGT TC AGA-GGT TGG CCT TAG GGT TC AGA-heg-F-5′) (SEQ ID NO: 5) 200 pmol, and (2) a plurality of detection probes and a target amplifier (3′-dT-AAA GAA ACT CCA. AAT CCT AAG-GGT TGG CCI TAG GGT TC AGA-GGT TGG CCT TAG GGT TC AGA-heg-F-5′) (SEQ ID NO: 5) 200 pmol and a plurality of detection probes (5′-CCA ACC GGA ATC CCA AG-heg-F(heghegheg-F)3-heg-dT-3′) (SEQ ID NO: 7) 400 pmol. The results are shown in FIG. 5. There was a ˜9 fold increase in fluorescence after hybridization of 4× amplifier (5′-CCA ACC GGA ATC CCA AG-heg-F(heghegheg-F)3-heg-H-3′; A03) (SEQ ID NO: 7).

### Example 6

### Branched Amplification

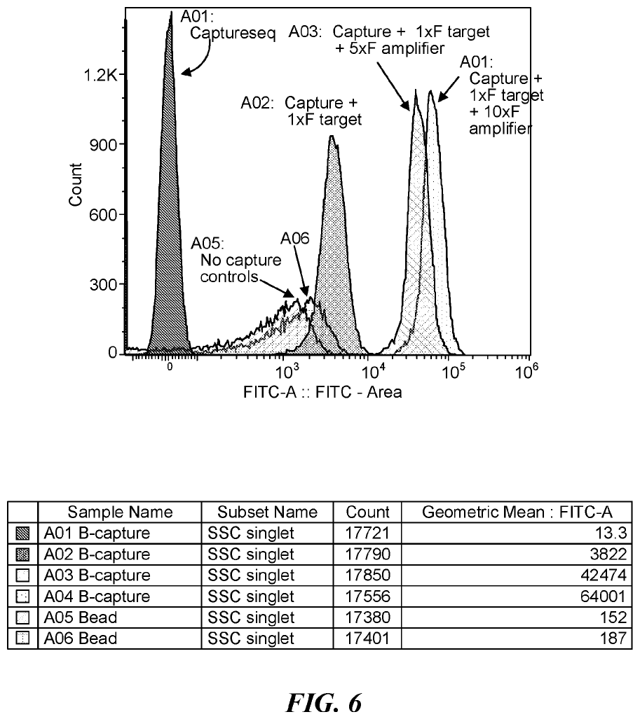

Samples are incubated at 37° C. for 10 minutes with a capture probe (A01); a capture probe+a target (3′-dT-AAA GAA ACT CCA AAT CCT AAG-GGT TGG CCT TAG GGT TC AGA-GGT TGG CCT TAG GGT TC AGA-heg-F-5′) (SEC) ID NO: 5)—(A02); a capture probe+a target (3′-dT-AAA GAA ACT CCA AAT CCI AAG-GGT TGG cur TAG GGT IC AGA-GGT TGG CCT TAG GGT TC AGA-heg-F-5′) (SEQ ID NO: 5)+5× amplifier (5′-CCA ACC GGA ATC CCA AG-heg-F(heghegheg-F)4-heg-dT-3′) (SEQ ID NO: 8)—(A03); a capture probe+a target (3′-dT-AAA GAA ACT CCA AAT CCT AAG-GGT TGG CCT TAG GGT TC AGA-GGT TGG CCT TAG GGT TC AGA-heg-F-5′) (SEQ ID NO: 5)+10× amplifier (5′-CCA ACC GGA ATC CCA. AG-heg-F(heghegheg-F)9-heg-dT-3′) (SEQ ID NO: 9)—(A04); a target (3′-dT-AAA GAA ACT CCA AAT CCT AAG-GGT TGG CCT TAG GGT TC AGA-GGT TGG CCT TAG GGT AGA-heg-F-5′) (SEQ ID NO: 5)+5× amplifier (5′-CCA ACC GGA ATC CCA AG-heg-F(heghegheg-F)4-heg-dl-3′) (SEQ ID NO: 8)—(A05); and a target (3′-dT-AAA GAA ACT CCA AAT CCT AAG-GGT TGG CCT TAG GGT TC AGA-GGT TGG CCT TAG GGT TC AGA-heg-F-5′) (SEQ ID NO: 5)+10× amplifier (5′-CCA ACC GGA ATC CCA AG-heg-F(heghegheg-F)9-heg-dT-3′) (SEQ ID NO: 9)—(A06). The results are shown in FIG. 6. There was a ˜11 fold increase in fluorescence after hybridization of 5× amplifier (5′-CCA ACC GGA ATC CCA AG-heg-F(heghegheg-F)4-heg-dT-3′) (SEQ IL) NO: 8) and a ˜16 fold increase in fluorescence after hybridization of 10× amplifier (5′-CCA. ACC GGA ATC CCA AG-heg-F(heghegheg-F)9-heg-dT-3′) (SEQ ID NO: 9).

### Example 7

### Concatemer Amplification

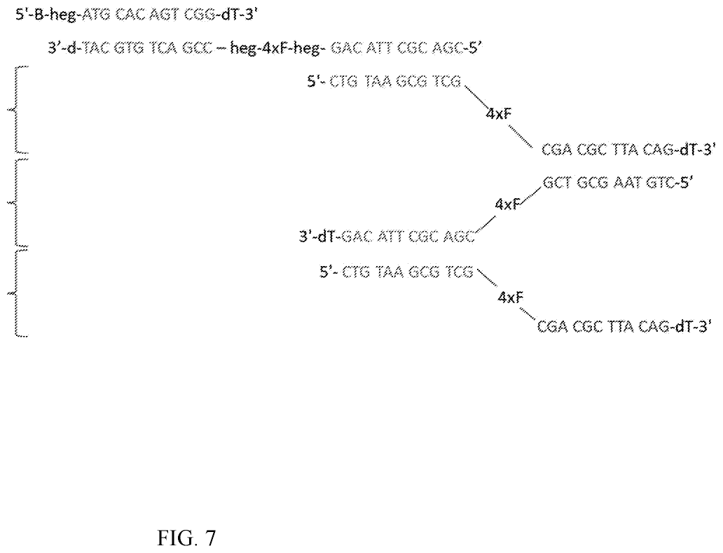

A biotinylated target probe (5′-B-heg-ATGCACAGTCGG-dT-3′) (SEQ ID NO: 1) bound to (3′-d-TACGTGTCAGCC-heg-F(heghegheg-F)3-heg-GACATTCGCAGC-5′) (SEQ ID NO: 10) are mixed with a second probe (5′-CTGTAAGCGTCG-heg-F(heghegheg-F)4-heg-CGACGCTTACAG-dT-3′) (SEQ. ID NO: 11), which concatemerizes in a zig-zag manner (as illustrated in FIG. 7). The resulting structure is then tested.

### Example 8

### Stepwise Linear Probe Hybridization in Solution

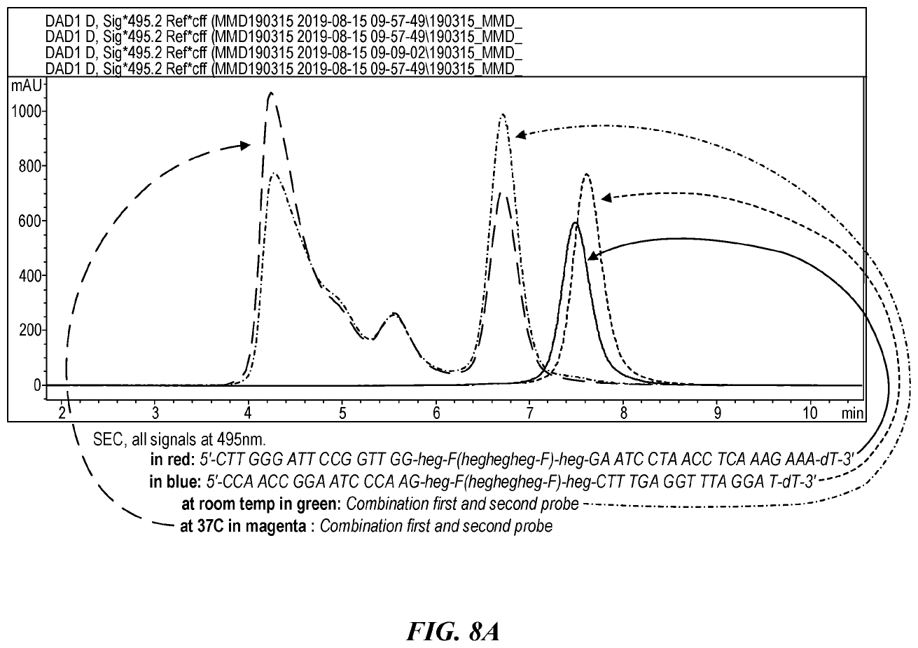

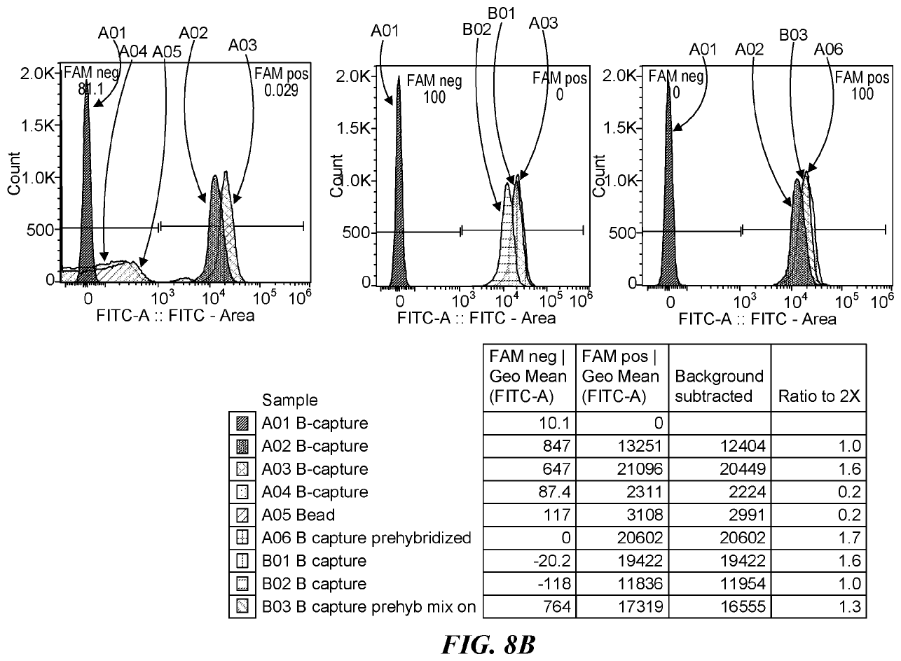

A first probe (3′-dT-AAA GAA ACT CCA ATC CTA AG-heg-F(heghegheg-F)-heg-GGT TGG CCT TAG GGT TC-5′) (SEQ ID NO: 12) was mixed with a second probe (5′-CCA ACC GGA ATC CCA AG-heg-F(heghegheg-F)-heg-CTT TGA GGT TTA GGA T-dT-3′) (SEQ ID NO: 13) under various conditions for hybridization (heat 200 uM stocks of each probe to 94° C. for 5 min, combine 1:1 for final concentration of 100 uM each). The results are shown in FIG. 8. Individual probes are found at ˜7.6m, hybridized probes start at ˜6.8 m (presumably single hybridization), and longer multimers are seen from 4.2-5.5 m, which are not completely resolved. Incubation at 37° C. resulted in longer chains.

Additionally, as shown in FIG. 8B, hybridization of the first probe to the second probe resulted in ˜1.6× increase in fluorescence. However, additional incubation with the first probe followed by the second probe did not increase fluorescence. Incubation with pre-hybridized material containing multimers from solution analysis did not show additional increase in fluorescence.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/118,544, filed on Nov. 25, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the following structure (II): 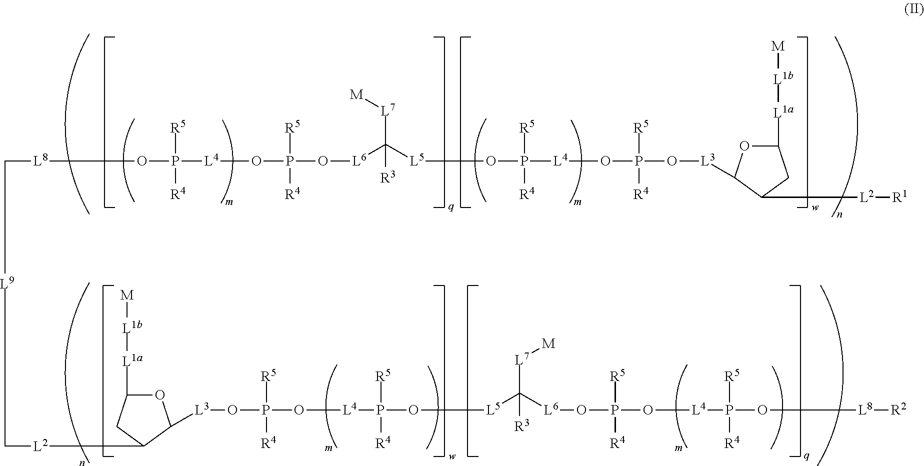
or a stereoisomer, salt or tautomer thereof, wherein:
M is, at each occurrence, independently a chromophore;
L^{1a} is, at each occurrence, independently a heteroarylene linker;
L² and L⁸ are independently optional linkers:
L^{1b}, L³, L⁵, L⁶ and L⁷ are, at each occurrence, independently optional alkylene, alkenylene, alkynylene, heteroalkylene, heteroalkenylene or heteroalkynylene linkers;
L⁴ is, at each occurrence, independently an alkylene, alkenylene, alkynylene, heteroalkylene, heteroalkenylene or heteroalkynylene linker;
L⁹ is a linker comprising a polynucleotide;
R¹ and R² are each independently H, OH, SH, alkyl, alkoxy, alkylether, heteroalkyl or —OP(═Rₐ)R_{b})R_{c};
R³ is, at each occurrence, independently H, alkyl or alkoxy;
R⁴ is, at each occurrence, independently OH, SH, O⁻, S⁻, OR_{d} or SR_{d};
R⁵ is, at each occurrence, independently oxo, thioxo or absent;
Rₐ i s O or S;
R_{b} is OH, SH, O⁻, S⁻, OR_{d} or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;
R_{d} is a counter ion;
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of one or greater; and
q and w are, at each occurrence, independently 0 or 1; provided at least one occurrence of either q or w is 1.

2. The compound of claim 1, wherein M is, at each occurrence, independently the same or different donor chromophore.

3. The compound of claim 1 or 2, wherein M is the same donor chromophore at each occurrence.

4. The compound of claim 1, wherein M is, at each occurrence, independently the same or different acceptor chromophore.

5. The compound of claim 1 or 4, wherein M is the same acceptor chromophore at each occurrence.

6. The compound of any one of claims 1-5, wherein M is, at each occurrence, independently the same or different fluorophore.

7. The compound of claim 6, wherein NI is, at each occurrence, independently the same or different donor fluorophore.

8. The compound or composition of claim 7, wherein M is the same donor fluorophore at each occurrence.

9. The compound of claim 6, wherein M is, at each occurrence, independently the same or different acceptor fluorophore.

10. The compound of claim 9, wherein N is the same acceptor fluorophore at each occurrence.

11. The compound of claim 1, wherein M is, at each occurrence, independently a dimethylaminostilbene, quinacridone, fluorophenyl-dimethyl-BODIPY, his-fluorophenyl-BUMPY, acridine, terrylene, sexiphenyl, porphyrin, benzopyrene, (fluorophenyl-dimethyl-difluorobora-diaza-indacene)phenyl, (bis-fluorophenyl-difluorobora-diaza-indacene)phenyl, quaterphenyl, bi-benzothiazole, ter-benzothiazole, bi-naphthyl, bi-anthracyl, squaraine, squarylium, 9, 10-ethynylanthracene or ter-naphthyl moiety.

12. The compound of claim 1, wherein M is, at each occurrence, independently pyrene, perylene, perylene monoimide, 5-FAM or 6-FAM or derivative thereof.

13. The compound of claim 1, wherein M, at each occurrence, independently has one of the following structures:

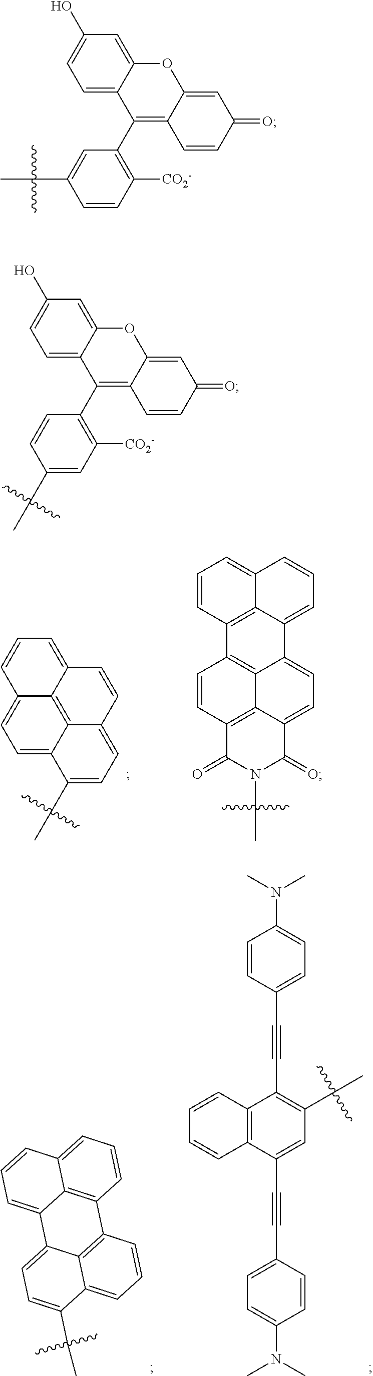

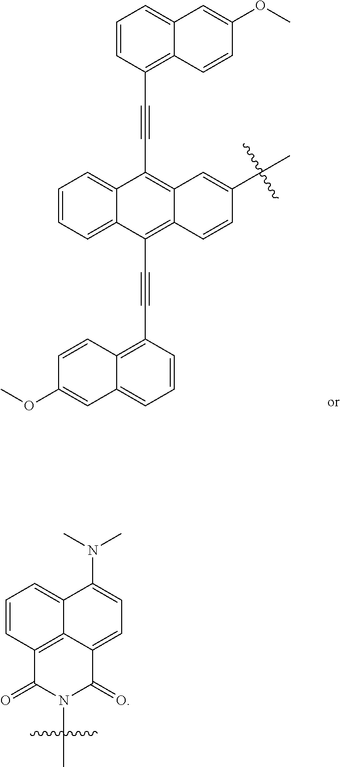

14. The compound of claim 1, wherein M, at each occurrence, independently has one of the following structures:

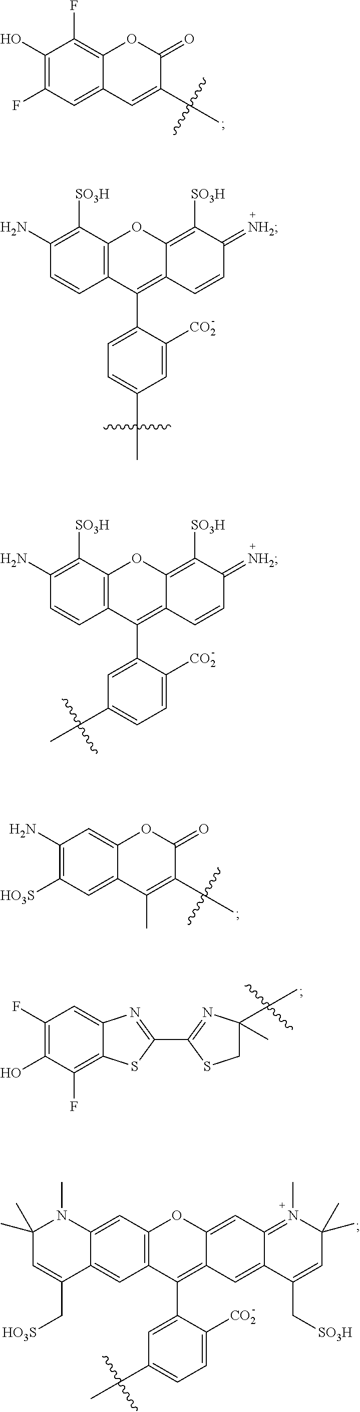

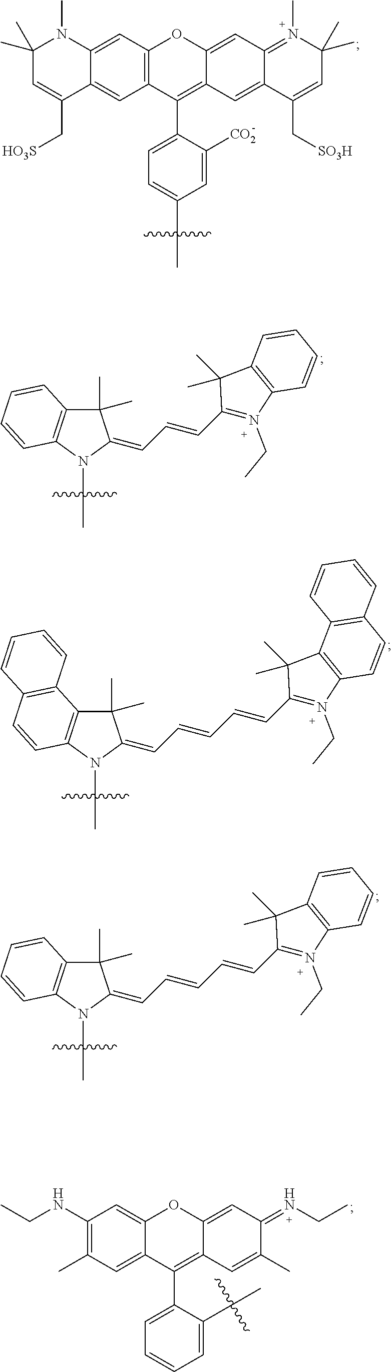

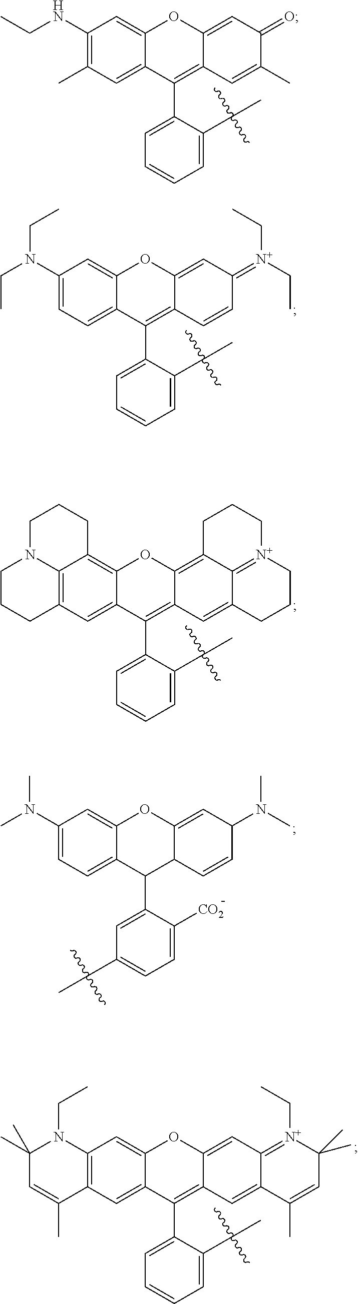

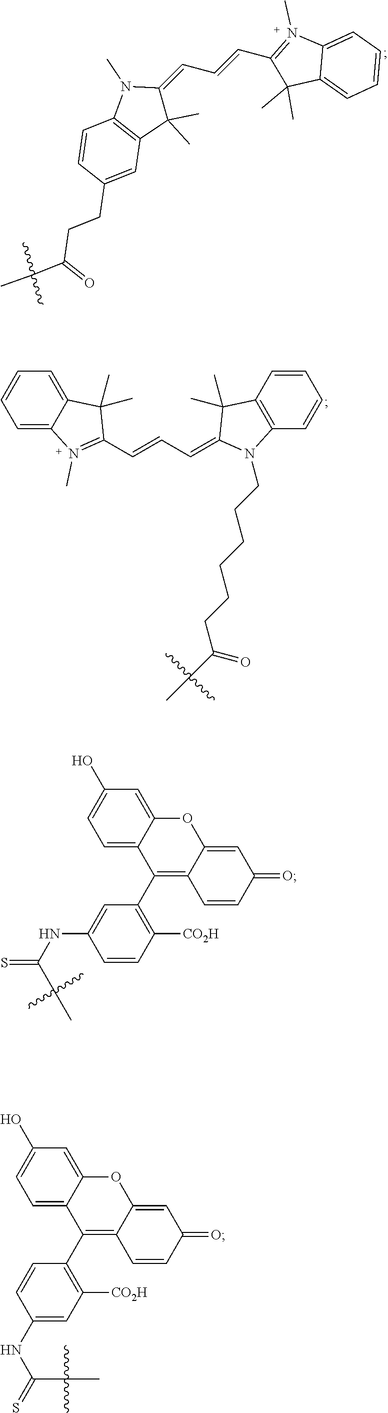

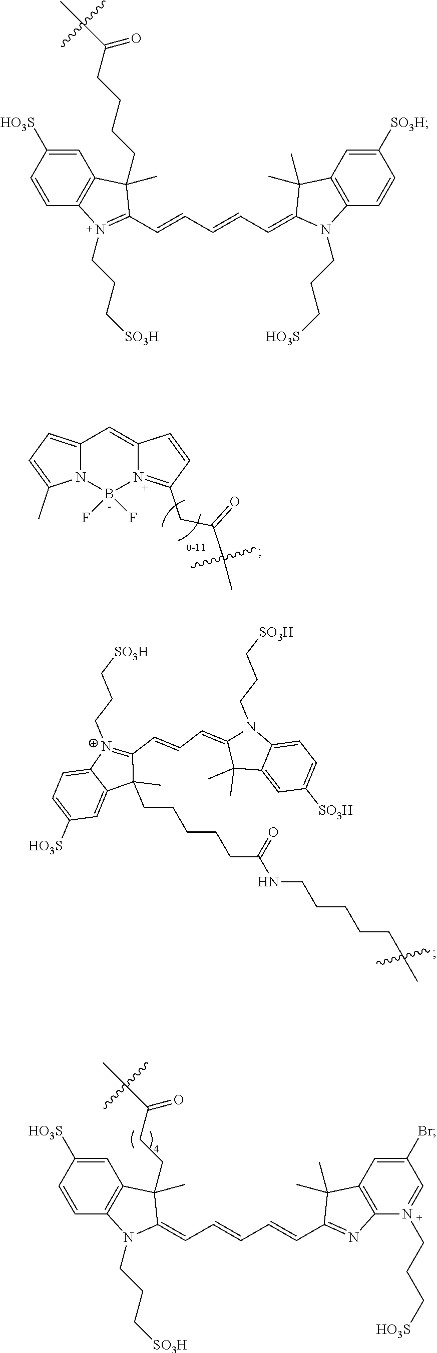

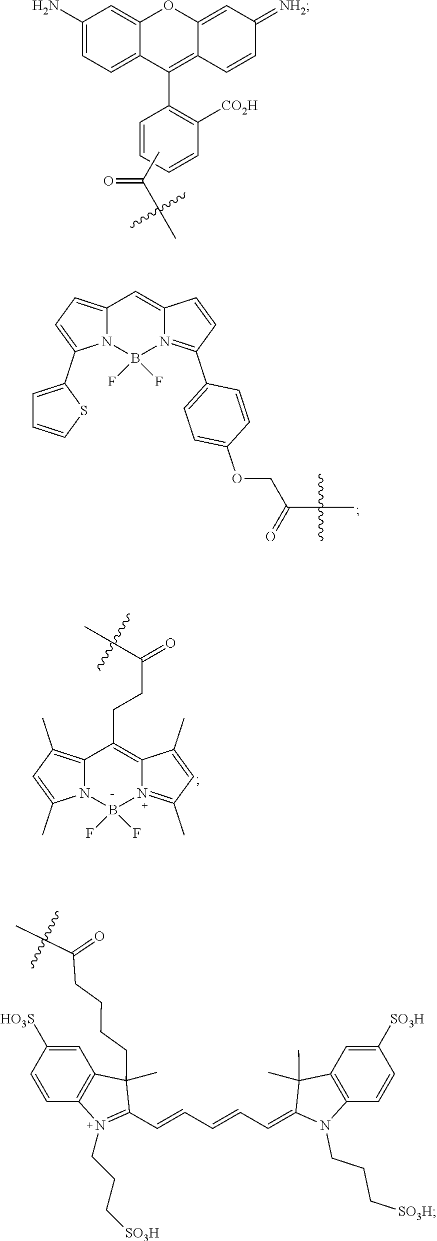

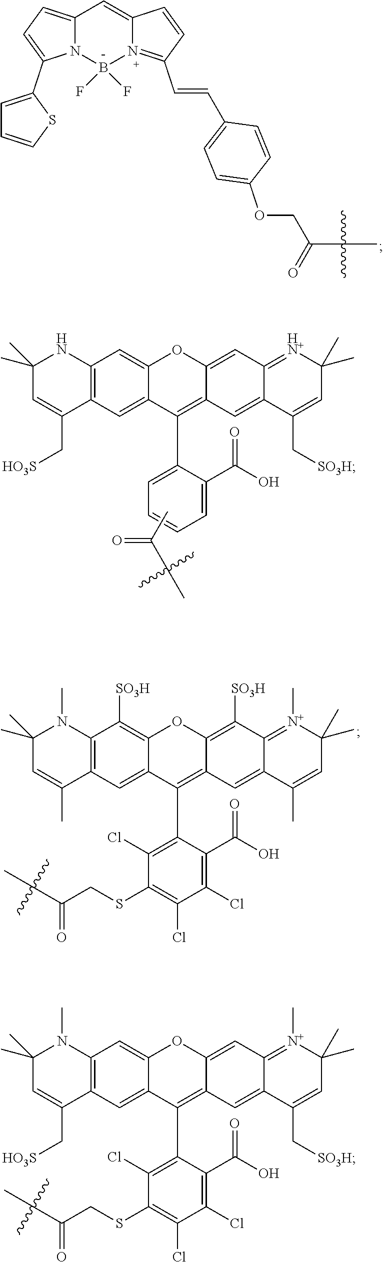

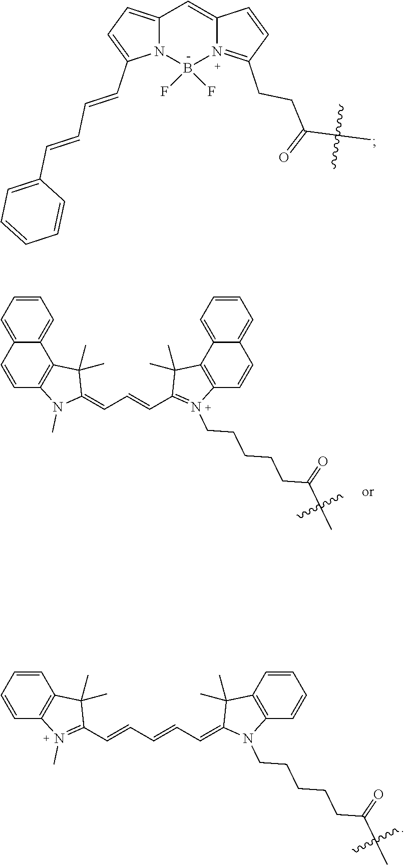

15. The compound of claim 1, having the following structure (IIa) or (IIb): 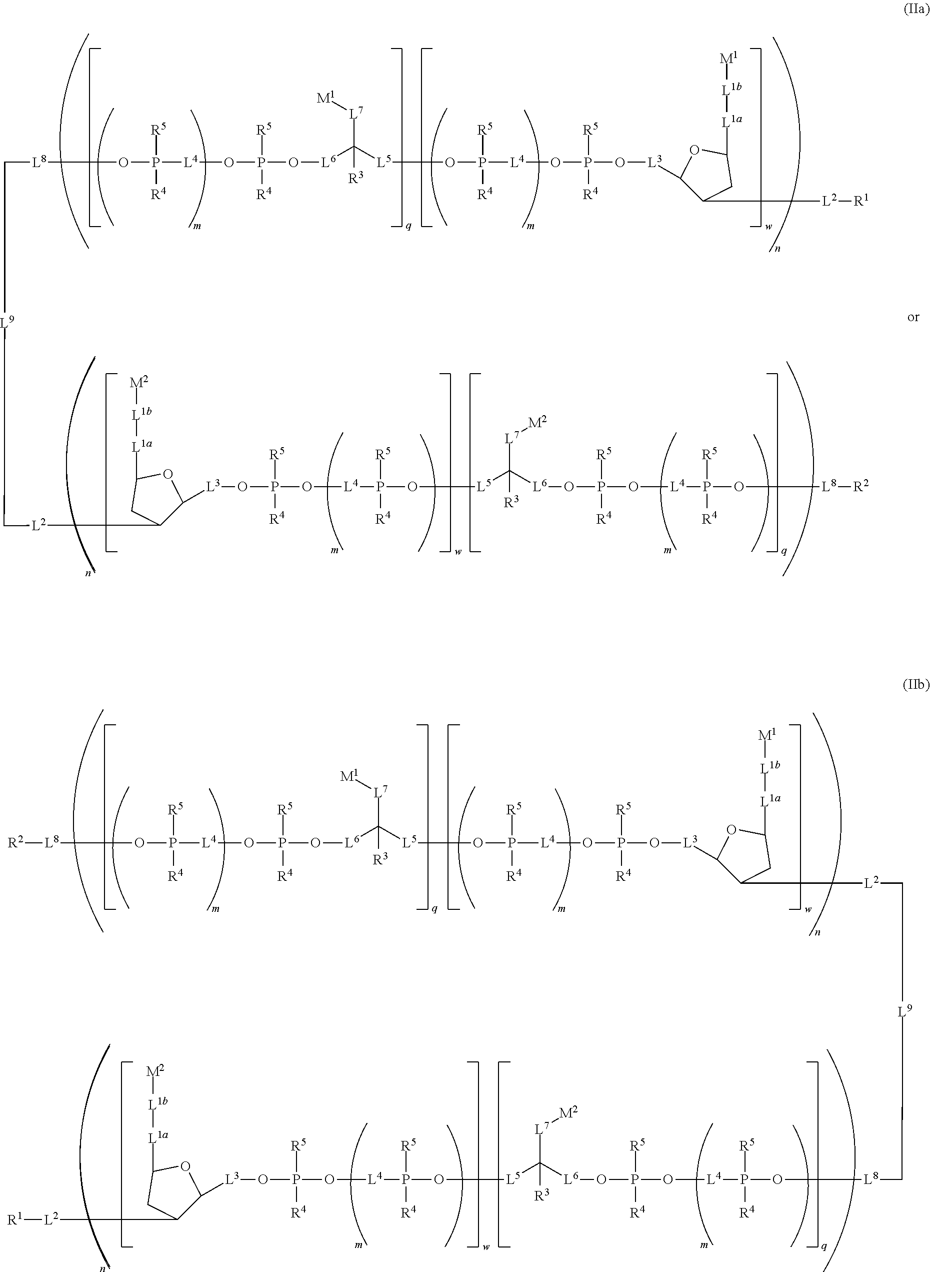
or a stereoisomer, salt or tautomer thereof; wherein:
M¹ is, at each occurrence, independently the same or different donor chromophores; and
M² at each occurrence, independently the same or different acceptor chromophores.

16. The compound of claim 15, having the following structure (IIa):

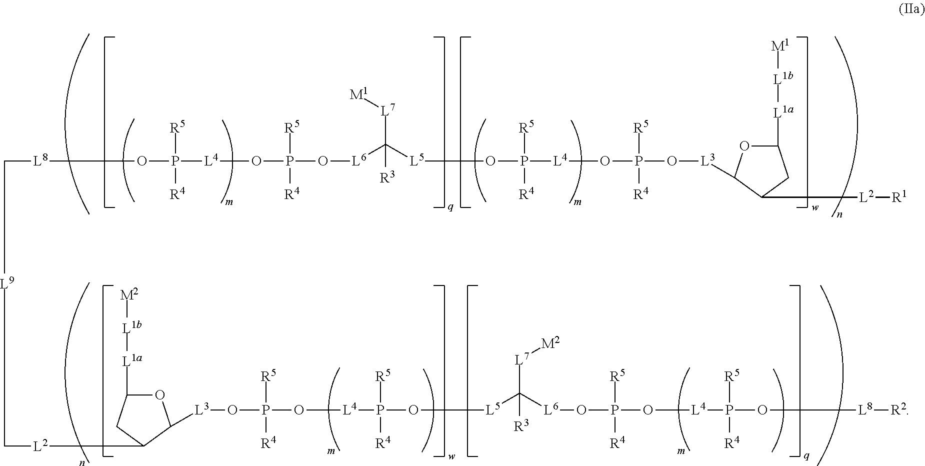

17. The compound of claim 15, having the following structure (IIb):

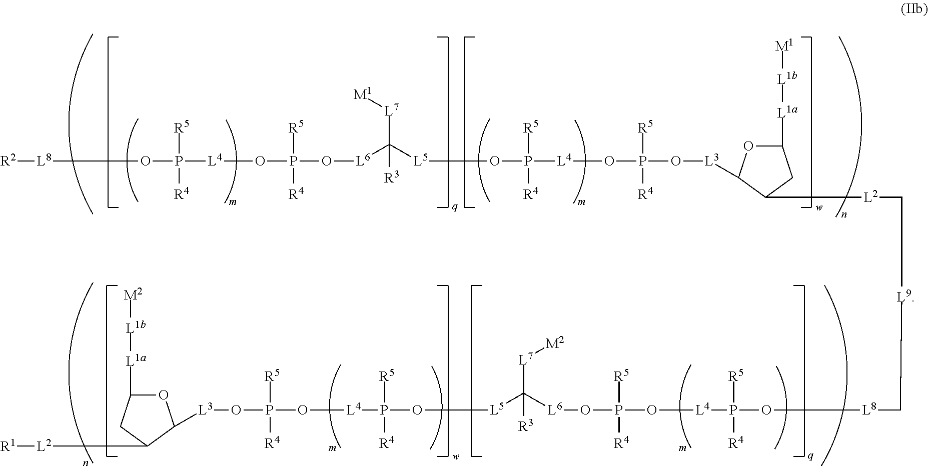

18. The compound of any one of claims 15-17, wherein M¹ is, at each occurrence, independently the same or a different donor fluorophore.

19. The compound of any one of claims 15-18, wherein M² is, at each occurrence, independently the same or a different acceptor fluorophore.

20. The compound of any one of claims 15-19, wherein M¹ is the same donor fluorophore at each occurrence.

21. The compound of any one of claims 15-20, wherein M² is the same acceptor fluorophore at each occurrence.

22. The compound of any one of claims 1-21, wherein the polynucleotide of L⁹ has a length ranging from 10 nucleotides to 40 nucleotides.

23. The compound of any one of claims 1-22, wherein the polynucleotide of L⁹ has a length ranging from 10 nucleotides to 30 nucleotides.

24. The compound of any one of claims 1-23, wherein the polynucleotide of L⁹ has a length ranging from 15 nucleotides to 25 nucleotides.

25. A compound having the following structure (III): 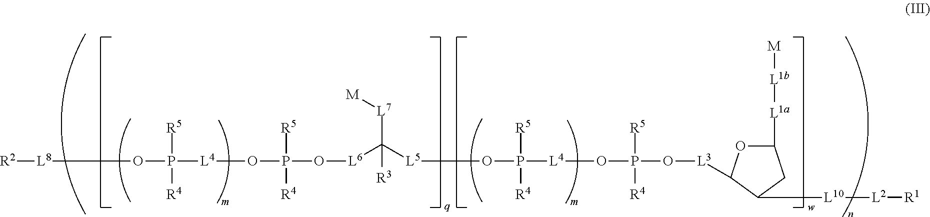
or a stereoisomer, salt or tautomer thereof, wherein:
M is, at each occurrence, independently the same or different chromophore;
L^{1a} is, at each occurrence, independently a heteroarylene linker;
L² and L⁸ are independently optional linkers;
L^{1b}, L³, L⁵, L⁶ and L⁷ are, at each occurrence, independently optional alkylene, alkenylene, alkynylene, heteroalkylene, heteroalkenylene or heteroalkynylene linkers;
L⁴ at each occurrence, independently an alkylene, alkenylene, alkynylene, heteroalkylene, heteroalkenylene or heteroalkynylene linker;
L¹⁰ is a linker comprising a polynucleotide;
R¹ and R² each independently H, OH, SH, alkyl, alkoxy, alkylether, heteroalkyl, —OP(—Rₐ)(R_{b})R_{c} or a moiety comprising a polynucleotide, provided at least one of R¹ and R² is a moiety comprising a polynucleotide;
R³ is, at each occurrence, independently H, alkyl or alkoxy;
R⁴ is, at each occurrence, independently OH, SH, O⁻, S⁻, OR_{d} or SR_{d};
R⁵ is, at each occurrence, independently oxo, thioxo or absent;
Rₐ is O or S;
R_{b} is OH, SH, O⁻, S⁻, OR_{d} or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;
R_{d} is a counter ion;
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of two or greater; and
q and w are, at each occurrence, independently 0 or 1, provided at least one occurrence of either q or w is 1.

26. The compound of claim 25, wherein R¹ comprises a polynucleotide.

27. The compound of claim 25 or 26, wherein R² comprises a polynucleotide.

28. The compound of claim 26 or 27, wherein R¹ comprises a targeting moiety bound to the polynucleotide.

29. The compound of claim 27 or 28, wherein R² comprises a targeting moiety bound to the poly nucleotide.

30. The compound of any one of claims 25-29, wherein M is, at each occurrence, independently the same or different donor chromophore.

31. The compound of claim 30, wherein M is the same donor chromophore at each occurrence.

32. The compound of any one of claims 25-29, wherein M is, at each occurrence, independently the same or different acceptor chromophore.

33. The compound of claim 32, wherein M is the same acceptor chromophore at each occurrence.

34. The compound of any one of claims 25-29, wherein M is, at each occurrence, independently the same or different fluorophore.

35. The compound claim 34, wherein M is, at each occurrence, independently the same or different donor fluorophore.

36. The compound of claim 34, wherein M is the same donor fluorophore at each occurrence.

37. The compound of claim 34, wherein M is, at each occurrence, independently the same or different acceptor fluorophore.

38. The compound of claim 37, wherein M is the same acceptor fluorophore at each occurrence.

39. The compound of any one of claims 25-29, wherein M is, at each occurrence, independently a dimethylaminostilbene, quinacridone, fluorophenyl-dimethyl-BODIPY, hi s-fluorophenyl-BODIPY, acridine, terrylene, sexiphenyl, porphyrin, benzopyrene, (fluorophenyl-dimethyl-difluorobora-diaza-indacene)phenyl, (hi s-fluorophenyl-difluorobora-diaza-indacene)phenyl, quaterphenyl, bi-benzothiazole, ter-benzothiazole, bi-naphthyl, bi-anthracyl, squaraine, squarylium, 9, 10-ethynylanthracene or ter-naphthyl moiety.

40. The compound of any one of claims 25-29, wherein M is, at each occurrence, independently pyrene, perylene, perylene monoimide, 5-FAM or 6-FAM or derivative thereof.

41. The compound of any one of claims 25-29, wherein M, at each occurrence, independently has one of the following structures:

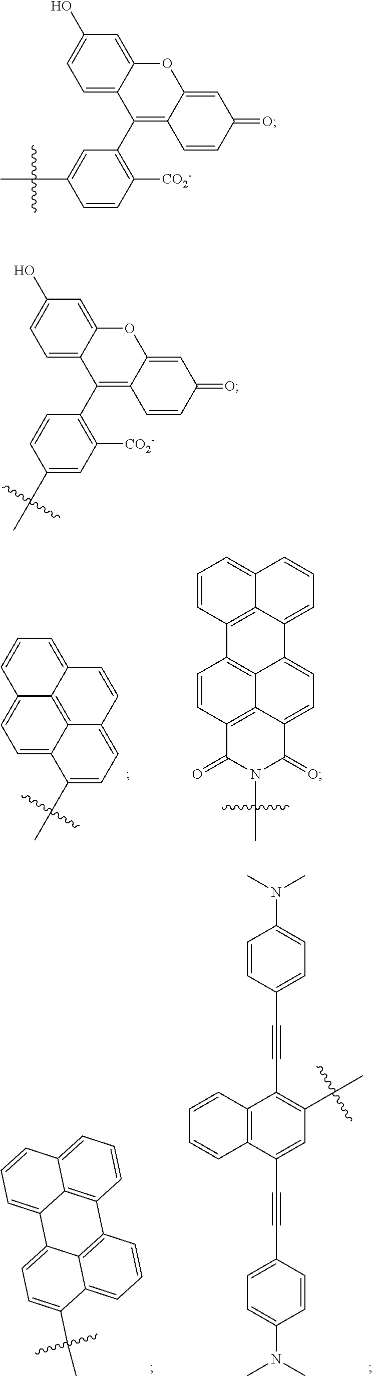

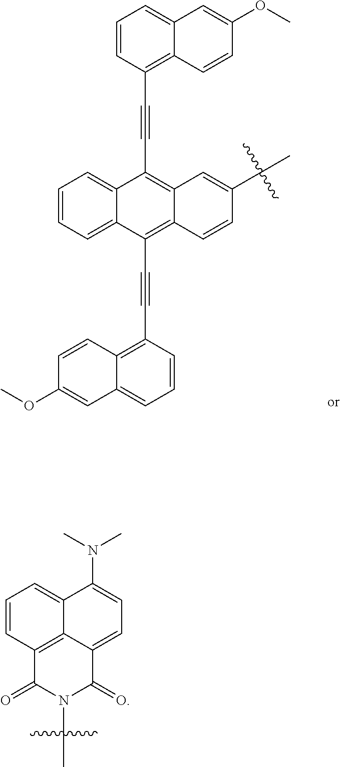

42. The compound of any one of claims 25-29, wherein M, at each occurrence, independently has one of the following structures:

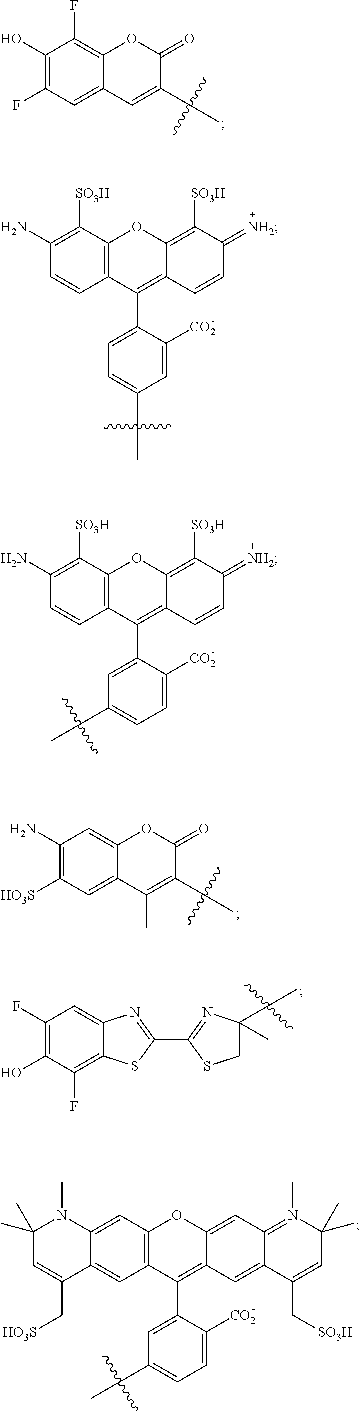

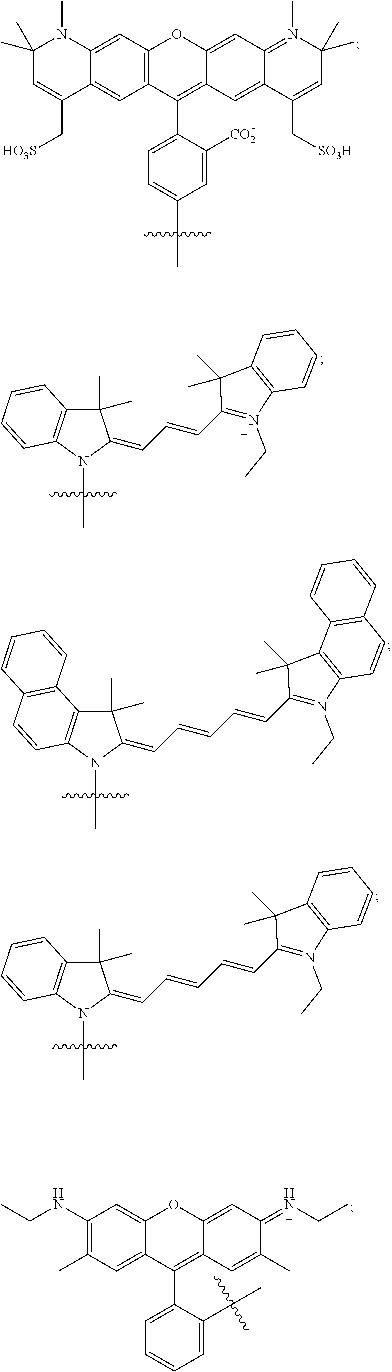

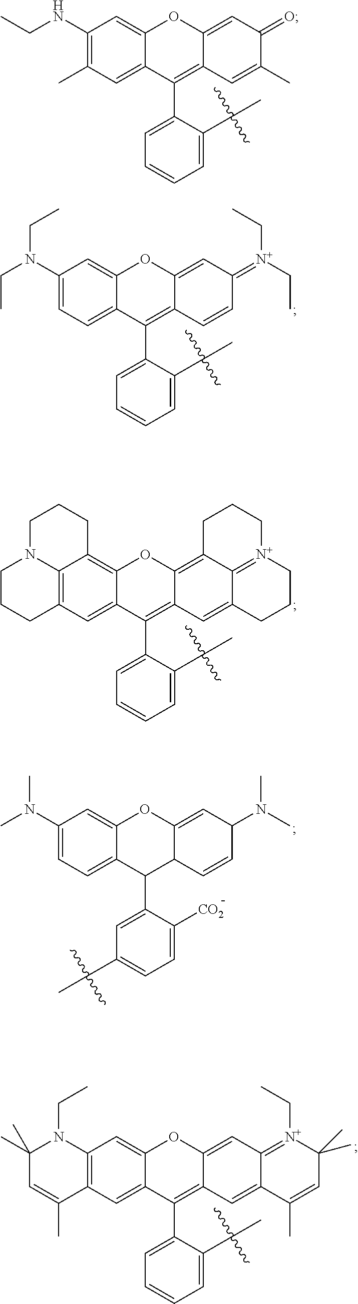

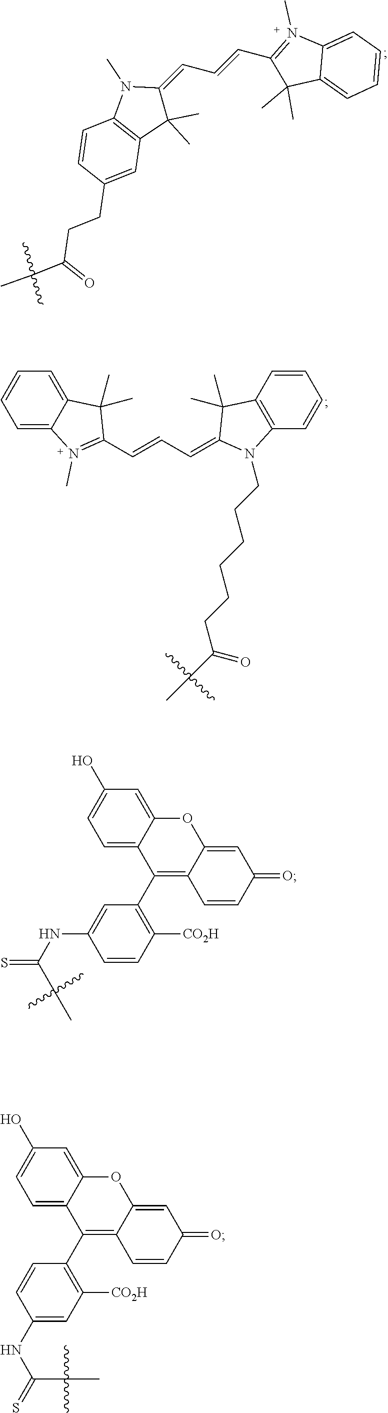

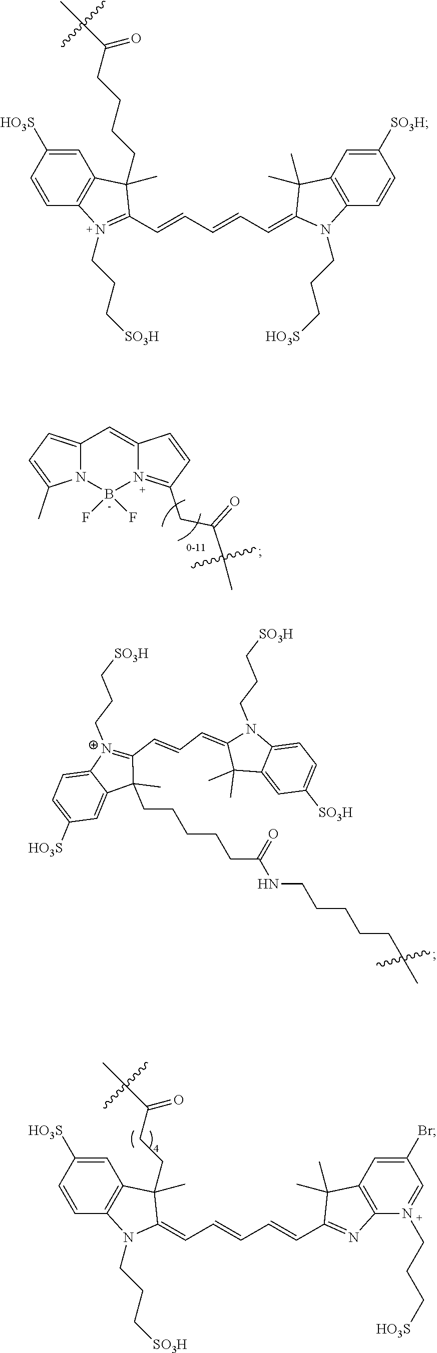

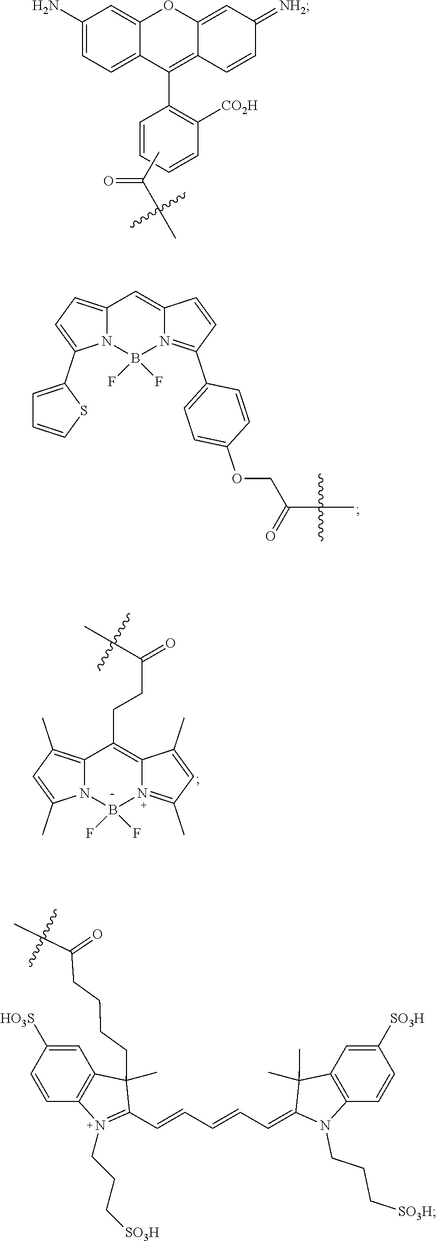

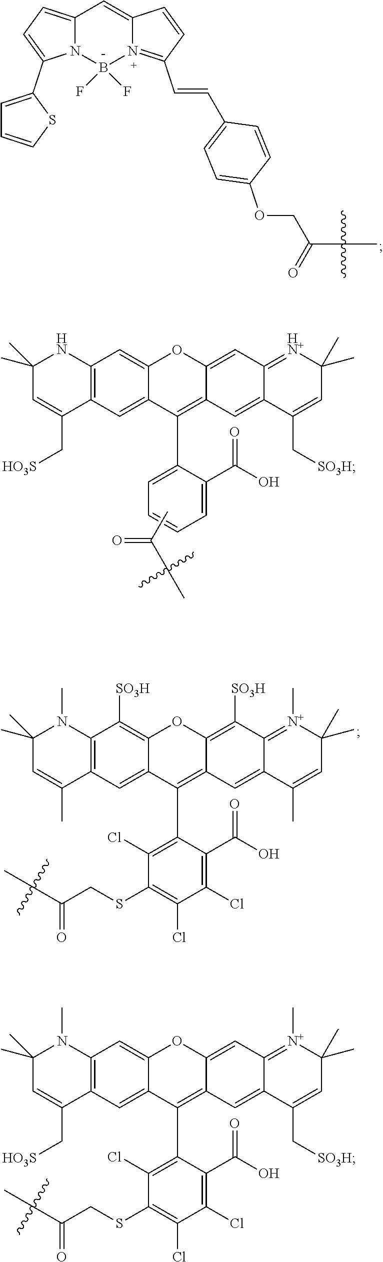

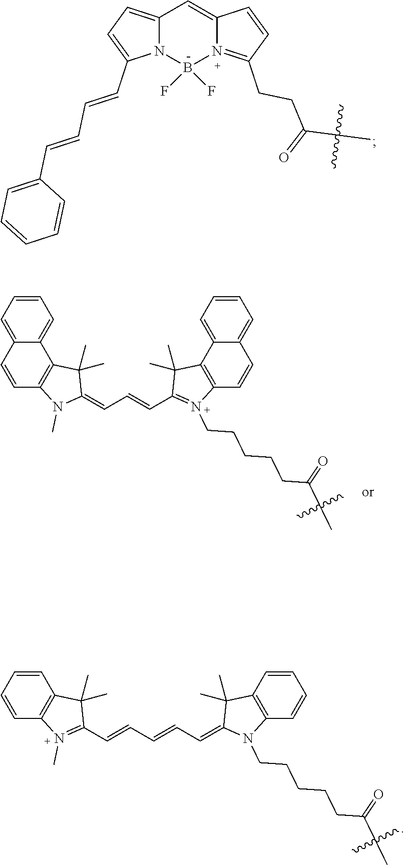

43. A composition comprising: the compound of any one of claims 1-24 and the compound of any one of claims 25-42.

44. The composition of claim 43, wherein the polynucleotide of L⁹ has a first sequence, the polynucleotide of L¹⁰ has a second sequence, the first sequence having at least 90% complementarity to at least a portion of the second sequence.

45. A composition comprising the compound of any one of claims 1-24, and a capture probe.

46. The composition of any one of claims 43-45, further comprising:
a compound having the following structure (I): 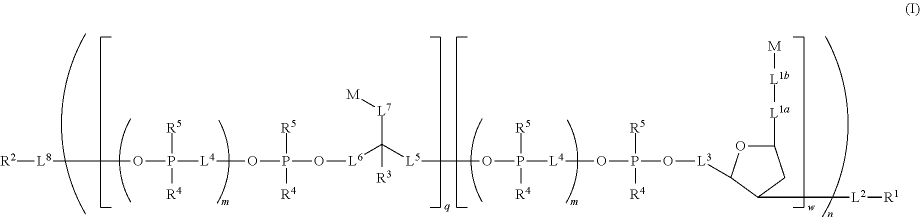
or a stereoisomer, salt or tautomer thereof, wherein:
M is, at each occurrence, independently the same or different chromophore;
L^{1a} is, at each occurrence, independently a heteroarylene linker;
L² and L⁸ are independently optional linkers;
L^{1b}, L³, L⁵, L⁶ and L⁷ are, at each occurrence, independently optional alkylene, alkenylene, alkynylene, heteroalkylene, heteroalkenylene or heteroalkynylene linkers;
L⁴ is, at each occurrence, independently an alkylene, alkenylene, alkynylene, heteroalkylene, heteroalkenylene or heteroalkynylene linker;
R¹ and R² each independently H, OH, SH, alkyl, alkoxy, alkylether, heteroalkyl, —OP(═Rₐ)(R_{b})R_{c} or a moiety comprising a polynucleotide, provided at least one of R¹ and R² is a moiety comprising a polynucleotide;
R³ is, at each occurrence, independently H, alkyl or alkoxy;
R⁴ is, at each occurrence, independently OH, SH, O⁻, S⁻, OR_{d} or SR_{d};
R⁵ is, at each occurrence, independently oxo, thioxo or absent;
Rₐ is O or S;
R_{b} is OH, SH, O⁻, S⁻, OR_{d} or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;
R_{d} is a counter ion;
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of one or greater; and
q and w are, at each occurrence, independently 0 or 1, provided at least one occurrence of either q or w is 1.

47. A composition comprising a capture probe and a compound having the following structure (I): 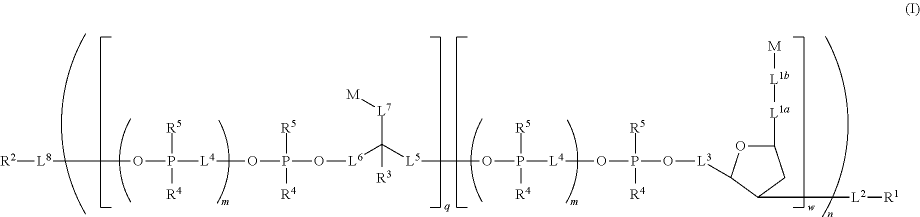
or a stereoisomer, salt or tautomer thereof, wherein:
M is, at each occurrence, independently the same or different chromophore;
at each occurrence, independently a heteroarylene linker;
L² and L⁸ are independently optional linkers;
L^{1b}, L³, L⁵, L⁶ and L⁷ are, at each occurrence, independently optional alkylene, alkenylene, alkynylene, heteroalkylene, heteroalkenylene or heteroalkynylene linkers;
L⁴ is, at each occurrence, independently an alkylene, alkenylene, alkynylene, heteroalkylene, heteroalkenylene or heteroalkynylene linker;
R¹ and R² each independently H, OH, SH, alkyl, alkoxy, alkylether, heteroalkyl, —OP(═Rₐ)(R_{b})R_{c} or a moiety comprising a polynucleotide, provided at least one of R¹ and R² is a moiety comprising a polynucleotide;
R³ is, at each occurrence, independently H, alkyl or alkoxy;
R⁴ is, at each occurrence, independently OH, SH, O⁻, S⁻, OR_{d} or SR_{d};
R⁵ is, at each occurrence, independently oxo, thioxo or absent;
Rₐ is O or S;
R_{b} is OH, SH, O⁻, S⁻, OR_{d} or SR_{d};
R_{c} is OH, SH, O⁻, S⁻, OR_{d}, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;
R_{d} is a counter ion;
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of one or greater; and
q and w are, at each occurrence, independently 0 or 1, provided at least one occurrence of either q or w is 1.

48. The composition of any one of claims 45-47, wherein the polynucleotide of R¹ or R² has a first sequence, the capture probe has a second sequence, the first sequence having at least 90% complementarity to at least a first portion of the second sequence.

49. The composition of any one of claims 45-47, further comprising a branched linker.

50. The composition of claim 49, wherein the branched linker comprises a first polynucleotide and a plurality of second polynucleotides.

51. The composition of claim 50, wherein the first polynucleotide of the branched linker has a first sequence and the capture probe has a second sequence, the first sequence having at least 90% complementarity to at least a first portion of the second sequence.

52. The composition of claim 50 or 51, wherein each of the second polynucleotides of the branched linker has a third sequence and the polynucleotide of R¹ or R² has a fourth sequence, the third sequence having at least 90% complementarity to the fourth sequence.

53. The composition of claim 52, wherein the third sequence having at least 92% complementarity to the fourth sequence.

54. The composition of claim 52 or 53, the third sequence having at least 95% complementarily to the fourth sequence.

55. The composition of any one of claims 52-54, the third sequence having at least 97% complementarily to the fourth sequence.

56. The composition of any one of claims 52-55, the third sequence having at least 98% complementarity to the fourth sequence.

57. The composition of any one of claims 52-56, the third sequence having at least 99% complementarity to the fourth sequence.

58. The composition of any one of claims 52-57, wherein the third sequence, the fourth sequence, or both have a length ranging from 10 nucleotides to 40 nucleotides.

59. The composition of any one of claims 52-58, wherein the third sequence, the fourth sequence, or both have a length ranging from 10 nucleotides to 30 nucleotides.

60. The composition of any one of claims 52-59, wherein the third sequence, the fourth sequence, or both have a length ranging from 15 nucleotides to 25 nucleotides.

61. The composition of any one of claims 45-60, wherein the capture probe comprises a targeting moiety.

62. The composition of claim 61, wherein the targeting moiety is an antibody.

63. A composition comprising the compound of any one of claims 25-42; and a compound having the following structure (I): 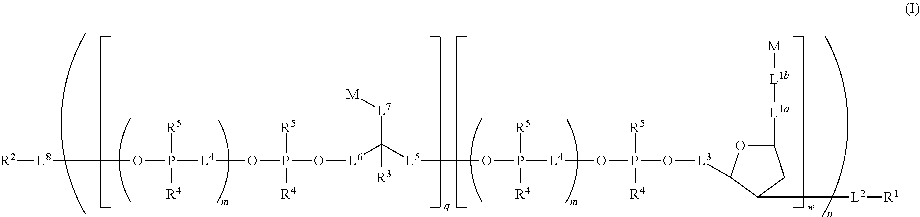
or a stereoisomer, salt or tautomer thereof, wherein:
M is, at each occurrence, independently the same or different chromophore;
L^{1a} at each occurrence, independently a heteroarylene linker;
L² and L⁸ are independently optional linkers;
L^{1b}, L³, L⁵, L⁶ and L⁷ are, at each occurrence, independently optional alkylene, alkenylene, alkynylene, heteroalkylene, heteroalkenylene or heteroalkynylene linkers;
L⁴ is, at each occurrence, independently an alkylene, alkenylene, alkynylene, heteroalkylene, heteroalkenylene or heteroalkynylene linker;
R¹ and R² each independently H, OH, SH, alkyl, alkoxy, alkylether, heteroalkyl, —OP(═R)R_{b})R_{c} or a moiety comprising a polynucleotide, provided at least one of R¹ and R² is a moiety comprising a polynucleotide;
R³ is, at each occurrence, independently H, alkyl or alkoxy;
R⁴ is, at each occurrence, independently OH, SH, O⁻, S⁻, OR_{d} or SR_{d};
R⁵ is, at each occurrence, independently oxo, thioxo or absent;
Rₐ is O or S;
R_{b} is OH, SH, O⁻, S⁻, OR_{d}, or SR_{d};
R_{c} is OH, SH, O⁻, S⁻, OR_{d}, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;
R_{d} is a counter ion;
L′ is, at each occurrence, independently a linker comprising a covalent bond to a solid support, a linker comprising a covalent bond to a solid support residue or a linker comprising a covalent bond to a nucleoside;
m is, at each occurrence, independently an integer of zero or greater;
n is an integer of one or greater; and
q and w are, at each occurrence, independently 0 or 1, provided at least one occurrence of either q or w is 1.

64. The composition of claim 63, wherein the polynucleotide of R¹ or R² has a first sequence, the L¹⁰ has a second sequence, the first sequence having at least 90% complementarity to at least a first portion of the second sequence.

65. The composition of any one of claims 44, 48, 51, or 64, wherein the first sequence having at least 92% complementarity to the portion of the first sequence.

66. The composition of any one of claims 44, 48, 51, 64, or 65, the first sequence having at least 95% complementarity to the portion of the first sequence.

67. The composition of any one of claims 44, 48, 51, or 64-66, the first sequence having at least 97% complementarity to the portion of the first sequence.

68. The composition of any one of claims 44, 48, 51, or 64-67, the first sequence having at least 98% complementarily to the portion of the first sequence.

69. The composition of any one of claims 44, 48, 51, or 64-68, the first sequence having at least 99% complementarily to the portion of the first sequence.

70. The composition of any one of claims 44, 48, 51, or 64-69, wherein the first sequence, the portion of the second sequence, or both have a length ranging from 10 nucleotides to 40 nucleotides.

71. The composition of any one of claims 44, 48, 51, or 64-70, wherein the first sequence, the portion of the second sequence, or both a length ranging from 10 nucleotides to 30 nucleotides.

72. The composition of any one of claims 44, 48, 51, or 64-71, wherein the first sequence, the portion of the second sequence, or both a length ranging from 15 nucleotides to 25 nucleotides.

73. The composition of any one of claims 44, 48, 51, or 64-72, wherein the second sequence comprises a second portion that is the same as the first portion.

74. The composition of any one of claims 44, 48, 51, or 64-73, further comprising a targeting moiety bound to a linking polynucleotide that has a third sequence that has at least 90% complementarity to at least a third portion of the second sequence.

75. The compound or composition of any one of claims 1-74, wherein each occurrence of q is 0.

76. The compound or composition of any one of claims 1-74, wherein at least one occurrence of q is 1.

77. The compound or composition of any one of claims 1-74 or 76, wherein each occurrence of w is 0.

78. The compound or composition of any one of claims 1-77, wherein at least one occurrence of w is 1.

79. The compound or composition of any one of claims 1-78, wherein at least one occurrence of L⁴ is heteroalkylene, or wherein each occurrence of L⁴ is heteroalkylene.

80. The compound or composition of claim 79, wherein the heteroalkylene comprises alkylene oxide.

81. The compound or composition of claim 80, wherein the heteroalkylene comprises ethylene oxide.

82. The compound or composition of any one of claims 79-81, wherein L⁴ has the following structure: 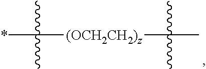
wherein:
z is an integer from 1 to 100; and
* indicates a bond to the adjacent phosphorous atom.

83. The compound or composition of claim 82, wherein z is an integer from 3 to 6 or an integer from 22 to 26.

84. The compound or composition of any one of claims 1-78, wherein at least one occurrence of L⁴ is alkylene, or wherein each occurrence of L⁴ is alkylene.

85. The compound or composition of claim 84, wherein at least one alkylene is ethylene, or wherein each alkylene is ethylene.

86. The compound or composition of any one of claims 1-85, wherein at least one occurrence of R³ is H, or wherein each occurrence of R³ is H.

87. The compound or composition of any one of claims 1-86, wherein L^{1a} is, at each occurrence independently an optionally substituted 5-7 membered heteroarylene linker.

88. The compound or composition of any one of claims 1-86, wherein L^{1a} has one of the following structures:

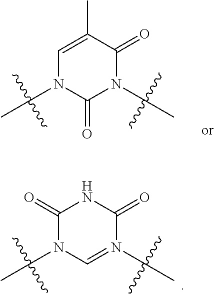

89. The compound or composition of any one of claims 1-88, wherein at least one occurrence of L³ is an alkylene linker, or wherein each occurrence of L³ is an alkylene linker.

90. The compound or composition of any one of claims 1-89, wherein at least one occurrence of L² and/or L⁸ is absent, or wherein each occurrence of L² and/or L⁸ is absent.

91. The compound or composition of any one of claims 1-90, wherein at least one occurrence of L⁵ and/or L⁶ is alkylene, or wherein each occurrence of L⁵ and/or L⁶ is alkylene.

92. The compound or composition of any one of claims 1-91, wherein L^{1b}, at each occurrence, independently comprises an amide functional group or a triazolyl functional group.

93. The compound or composition of any one of claims 1-92, wherein R⁵ is, at each occurrence, independently OH, O⁻ or OR_{d}.

94. The compound or composition of any one of claims 1-93, wherein R⁴ is, at each occurrence, oxo.

95. The compound or composition of any one of claims 1-94, wherein at least one occurrence of L⁷ is an optionally substituted heteroalkylene linker, or wherein each occurrence of L⁷ is independently an optionally substituted heteroalkylene linker.

96. The compound or composition of any one of claims 1-95, wherein L⁷ comprises an amide or a triazolyl functional group.

97. The compound or composition of any one of claims 1-96, wherein each occurrence of 1⁷ has one of the following structures:

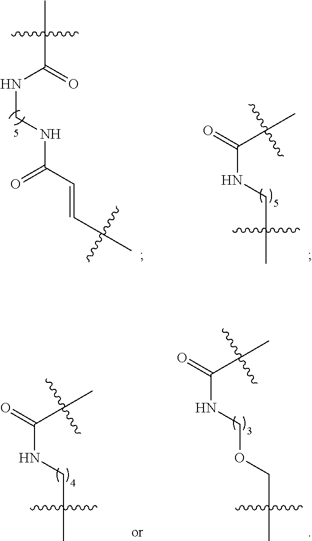

98. The compound or composition of any one of claims 1-96, wherein n is an integer from 1 to 100, or wherein n is an integer from 1 to 10.

99. The compound or composition of any one of claims 1-98, wherein in is an integer from 3 to 6, or wherein m is 3.

100. A composition; comprising:
a capture probe comprising:
a first segment comprising a first polynucleotide having a first sequence; and
a second segment conjugated to the first segment, the second segment comprising a plurality of alternating second polynucleotides and first polymers, each of the first polymers comprising a first chromophore, each of the second polynucleotides comprising a second sequence; and
a plurality of detectable probes, each of the plurality of detectable probes comprising a third polynucleotide covalently bound to at least one a second polymer comprising a second chromophore, the third polynucleotide having a third sequence that has at least 90% complementarity to the second sequence.

101. The composition of claim 100, wherein each of the first polymers comprise a plurality of the first chromophores.

102. The composition of claim 100 or 101, wherein each of the detectable probes comprise a plurality of the second chromophores.

103. The composition of any one of claims 100-102, wherein the first chromophore is an acceptor chromophore.

104. The composition of any one of claims 100-102, wherein the first chromophore is a donor chromophore.

105. The composition of any one of claims 100-104, wherein the second chromophore is an acceptor chromophore.

106. The composition of any one of claims 100-104, wherein the second chromophore is a donor chromophore.

107. The composition of any one of claims 100-106, wherein the first chromophore is a fluorophore.

108. The composition of any one of claims 100-107, wherein the second chromophore is a fluorophore.

109. The composition of any one of claims 100-108, wherein a respective second polymer is covalently bound to each end of the third polynucleotide of at least one of the detectable probes of the plurality of detectable probes.

110. A composition, comprising:
a capture probe comprising:
a first segment comprising a first polynucleotide having a first sequence; and
a second segment conjugated to the first segment, the second segment comprising a plurality of second polynucleotides each comprising a second sequence; and
a plurality of detectable probes, each of the plurality of detectable probes comprising a third polynucleotide having a first end and a second end, the first end being covalently bound to a first polymer comprising an acceptor chromophore, the second end being covalently bound to a second polymer comprising a donor chromophore, the third polynucleotide having a third sequence that has at least 90% complementarity to the second sequence.

111. The composition of any one of claims 100-110, wherein the capture probe has the structure of any one of claims 25-42.

112. The composition of any one of claims 100-111, wherein at least one of the detectable probes is, independently the compound of any one of claims 1-24.

113. The composition of any one of claims 100-112, further comprising a fourth polynucleotide covalently bound to a targeting moiety, the fourth polynucleotide having a fourth sequence that has at least 90% complementarity to the first sequence.

114. The composition of any one of claims 100-113, wherein the fourth sequence has at least 92% complementarity to the first sequence.

115. The composition of any one of claims 100-114, wherein the fourth sequence has at least 95% complementarity to the first sequence.

116. The composition of any one of claims 100-115, wherein the fourth sequence has at least 96% complementarity to the first sequence.

117. The composition of any one of claims 100-116, wherein the fourth sequence has at least 97% complementarity to the first sequence.

118. The composition of any one of claims 100-117, wherein the fourth sequence has at least 98% complementarity to the first sequence.

119. The composition of any one of claims 100-118, wherein the fourth sequence has at least 99% complementarity to the first sequence.

120. The composition of any one of claims 100-119, wherein the second sequence has at least 92% complementarily to the third sequence.

121. The composition of any one of claims 100-120, wherein the second sequence has at least 95% complementarity to the third sequence.

122. The composition of any one of claims 100-121, wherein the second sequence has at least 96% complementarity to the third sequence.

123. The composition of any one of claims 100-122, wherein the second sequence has at least 97% complementarity to the third sequence.

124. The composition of any one of claims 100-123, wherein the second sequence has at least 98% complementarity to the third sequence.

125. The composition of any one of claims 100-124, wherein the second sequence has at least 99% complementarity to the third sequence.

126. The composition of any one of claims 110-125, wherein the acceptor chromophore is a fluorophore.

127. The composition of any one of claims 110-126, wherein the donor chromophore is a fluorophore.

128. The composition of any one of claims 100-127, wherein the capture probe comprises a targeting moiety.

129. The composition of claim 123, wherein the targeting moiety is an antibody.

130. A kit comprising the compound or composition of any one of claims 1-129 and instructions for use.

131. A method for identifying the presence of a target analyte, comprising:
producing a mixture by contacting a sample with the composition of any one of claims 43-130 under assay conditions; and
imaging the mixture under detection conditions.

132. The method of claim 131, wherein the imaging the mixture comprises:
exciting the donor fluorophores at a first wavelength; and
detecting emission of the acceptor fluorophores at a second wavelength.
